(19) United States
(12) Patent Application Publication
(10) Pub. No.: US 2006/0142336 A1
(43) Pub. Date: Jun. 29, 2006

(54) N-sulfonylpiperidines as metalloproteinase inhibitors (tace)

(76) Inventors: Jeremy Nicholas Burrows, Alderley Park (GB); Howard Tucker, Alderely Park (GB)

Correspondence Address: FISH & RICHARDSON P.C., P.O BOX 1022, MINNEAPOLIS, MN, 55440-1022, US

(21) Appl. No.: 10/521,042

(22) PCT Filed: Jul. 9, 2003
(86) PCT No.: PCT/GB03/02959

§ 371 Date: Dec. 7, 2005

(30) Foreign Application Priority Data
Jul. 13, 2002 (GB) 0216379.8

(51) Int. Cl. A61K 31/4545; A61K 31/445; C07D 211/68; C07D 401/02

(52) U.S. Cl. 514/317; 514/318; 546/194; 546/229

(57) ABSTRACT

Compounds of formula (1), wherein Z is —CONR₁₅OH or —N(OH)CHO and X is —(CR₉R₁₀)t-Q-(CR₁₁R₁₂)u- (where t and u are independently 0 or 1 with the proviso that t and u cannot both be 0);are inhibitors of metalloproteinases and in particular TACE.

The present invention relates to compounds useful in the inhibition of metalloproteinases and in particular to pharmaceutical compositions comprising them, as well as their use.

The compounds of this invention are inhibitors of one or more metalloproteinase enzymes and are particularly effective as inhibitors of TACE (TNFα Converting Enzyme). Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMP) such as the collagenases (MMP1, MMP8, MMP13), the gelatinases (MMP2, MMP9), the stromelysins (MMP3, MMP10, MMP11), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biologically important cell mediators, such as tumour necrosis factor (TNF); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many disease conditions. Inhibition of the activity of one or more metalloproteinases may well be of benefit in these disease conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema and dermatitis); in tumour metastasis or invasion; in disease associated with uncontrolled degradation of the extracellular matrix such as osteoarthritis; in bone resorptive disease (such as osteoporosis and Paget's disease)); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; and extracellular matrix remodelling observed in cardiovascular diseases such as restenosis and atheroscelerosis.

A number of metalloproteinase inhibitors are known; different classes of compounds may have different degrees of potency and selectivity for inhibiting various metalloproteinases. We have discovered a class of compounds that are inhibitors of metalloproteinases and are of particular interest in inhibiting TACE. The compounds of this invention have beneficial potency and/or pharmacokinetic properties.

TACE (also known as ADAM17) which has been isolated and cloned [R. A. Black et al. (1997) Nature 385:729-733; M. L. Moss et al. (1997) Nature 385:733-736] is a member of the admalysin family of metalloproteins. TACE has been shown to be responsible for the cleavage of pro-TNFα, a 26 kDa membrane bound protein to release 17 kDa biologically active soluble TNFα [Schlondorff et al. (2000) Biochem. J. 347: 131-138]. TACE mRNA is found in most tissues, however TNFα is produced primarily by activated monocytes, macrophages and T lymphocytes. TNFα has been implicated in a wide range of pro-inflammatory biological processes including induction of adhesion molecules and chemokines to promote cell trafficking, induction of matrix destroying enzymes, activation of fibroblasts to produce prostaglandins and activation of the immune system [Aggarwal et al (1996) Eur. Cytokine Netw. 7: 93-124]. Clinical use of the anti-TNF biologicals has shown TNFα to play an important role in a range of inflammatory diseases including rheumatoid arthritis, Crohn's disease and psoriasis [Onrust et al (1998) Biodrugs 10: 397-422, Jarvis et al (1999) Drugs 57:945-964]. TACE activity has also been implicated in the shedding of other membrane bound proteins including TGFα, p75 & p55 TNF receptors, L-selectin and amyloid precursor protein [Black (2002) Int. J. Biochem. Cell Biol. 34: 1-5]. The biology of TACE inhibition has recently been reviewed and shows TACE to have a central role in TNFα production and selective TACE inhibitors to have equal, and possibly greater, efficacy in the collagen induced arthritis model of RA than strategies that directly neutralise TNFα [Newton et al (2001) Ann. Rheum. Dis. 60: iii25-iii32].

A TACE inhibitor might therefore be expected to show efficacy in all disease where TNFα has been implicated including, but not limited to, inflammatory diseases including rheumatoid arthritis and psoriasis, autoimmune diseases, allergic/atopic diseases, transplant rejection, graft versus host disease, cardiovascular disease, reperfusion injury and malignancy.

Compounds that inhibit matrix metalloproteinases are already known in the art. WO 00112477 discloses hydroxamic acids and carboxylic acid derivatives that are inhibitors of matrix metalloproteinases; WO 00/12478 discloses arylpiperazines that are useful in the inhibition of matrix metalloproteinase and are of particular interest as regards the inhibition of MMP13 and MMP9; and WO 01/87870 discloses hydroxamic acid derivatives which are inhibitors of matrix metalloproteinases including ADAM or ADAM-TS enzymes.

Surprisingly we have discovered that a selection of compounds are very potent inhibitors of TACE (ADAM17) and are particularly noteworthy for their unexpected selectivity for TACE over the matrix metalloproteinases

Additionally further effective compounds are disclosed.

According to one aspect of the present invention there is provided a compound of formula (1): 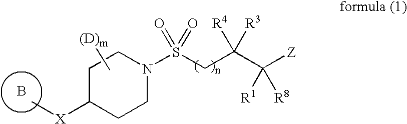
wherein Z is selected from —CONR¹⁵OH and —N(OH)CHO;

R¹⁵ is hydrogen or C₁₋₃alkyl;

wherein R¹ is hydrogen or a group selected from C₁₋₆alkyl, C₂₋₆alkenyl, C₂₋₆alkyl, C₃₋₇cycloalkyl, C₅₋₇cycloalkenyl, aryl, heteroaryl and heterocyclyl where the group is optionally substituted by one or more substituents independently selected from halo, nitro, cyano, trifluoromethyl, trifluoromethyloxy, C₁₋₄alkyl, C₂₋₄alkenyl, C₇₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, C₁₋₄alkoxycarbonyl, —OR⁵, —SR², —SOR², —SO₂R², —COR², —CONR⁵R⁶, —NR¹⁶COR⁵, —OSR⁵R⁶ and —NR¹⁶SO₂R²;

R¹⁶ is hydrogen or C₁₋₃alkyl;

R¹⁷ is selected from halo, C₁₋₆alkyl, C₃₋₆cycloalkyl and C₁₋₆alkoxy;

R² is group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;

R⁵ is hydrogen or a group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;

R⁶ is hydrogen, C₁₋₆alkyl or C₃₋₆cycloalkyl;

or R⁵ and R⁶ together with the nitrogen to which they are attached form a heterocyclic 4- to 7-membered ring;

wherein R⁸ is hydrogen or a group selected from C₁₋₆alkyl, C₃₋₇cycloalkyl, C₅₋₇cycloalkenyl and heterocyclyl where the group is optionally substituted by one or more substituents independently selected from halo, nitro, cyano, trifluoromethyl, trifluoromethyloxy and C₁₋₄alkyl;

or R¹ and R⁸ together form a carbocyclic or saturated heterocyclic 3- to 6-membered ring;

wherein R³ and R⁴ are independently hydrogen, C₁₋₆alkyl, C₃₋₆cycloalkyl, C₅₋₇cycloalkenyl, heterocyclyl, aryl or heteroaryl;

wherein n is 0 or 1;

wherein m is 0 or 1;

wherein D is hydrogen, C₁₋₄alkyl, C₃₋₆cycloalkyl or fluoro;

wherein X is —(CR⁹R¹⁰)ₜQ-(CR¹¹R¹²)ᵤ— where t and u are independently 0 or 1 with the proviso that t and u cannot both be 0;

wherein Q is O, S, SO or SO₂;

R⁹, R¹⁰, R¹¹ and R¹² are independently selected from hydrogen, C₁₋₄alkyl and C₃₋₆cycloalkyl;

wherein B is a group selected from aryl, heteroaryl, heterocyclyl, C₃₋₁₀cycloalkyl and C₅₋₇cycloalkenyl where each group is optionally substituted by one or more groups independently selected from nitro, trifluoromethyl, trifluoromethyloxy, halo, C₁₋₄alkyl (optionally substituted by one or more R¹³), C₂₋₄alkenyl, C₂₋₄alkynyl, C₃₋₆cycloalkyl (optionally substituted by one or more R¹³), heterocycloalkyl, heteroaryl, —OR¹³, cyano, —NR¹³R¹⁴, —CONR¹³R¹⁴, —NR⁶COR¹³, —SO₂NR¹³R¹⁴, —NR¹⁶SO₂R¹³, —SR³, —SOR⁷ and —SO₂R⁷;

R⁷ is C₁₋₆alkyl or C₃₋₆cycloalkyl

R¹³ and R¹⁴ are independently hydrogen, C₁₋₆alkyl or C₃₋₆cycloalkyl;

or R¹³ and R¹⁴ together with the nitrogen to which they are attached form a heterocyclic 4 to 7-membered ring.

In a preferred embodiment of the invention:

Z is selected from —CONR¹⁵OH and —N(OH)CHO;

R¹⁵ is hydrogen or C₁₋₃alkyl;

R¹ is hydrogen or a group selected from C₁₋₆alkyl, C₂₋₆alkenyl, C₂₋₆alkenyl, C₃₋₇cycloalkyl, C₅₋₇cycloalkenyl, aryl and heteroaryl where the group is optionally substituted by one or more substituents independently selected from halo, nitro, cyano, trifluoromethyl, trifluoromethyloxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, C₁₋₄alkoxycarbonyl, —OR⁵, —SR², —OR², —SOR², —SO₂R², —COR², —CO₂R⁵, —CONR⁵R⁶, —NR¹⁶COR⁵, —SO₂NR⁵R⁶ and —NR¹⁶SO₂R²;

R¹⁶ is hydrogen or C₁₋₃alkyl;

R¹⁷ is selected from halo, nitro, cyano, trifluoromethyl, trifluoromethoxy, C₁₋₆alkyl, C₃₋₆cycloalkyl and C₁₋₆alkoxy;

R² is group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;

R⁵ is hydrogen or a group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;

R⁶ is hydrogen, C₁₋₆alkyl or C₃₋₆cycloalkyl;

or R⁵ and R⁶ together with the nitrogen to which they are attached form a heterocyclic 4- to 7-membered ring;

R⁵ is hydrogen or a group selected from C₁₋₆alkyl, C₃₋₇cycloalkyl and C.₅-cycloalkenyl where the group is optionally substituted by one or more substituents independently selected from halo, nitro, cyano, trifluoromethyl, trifluoromethyloxy and C₁₋₄alkyl;

R³ and R⁴ are both hydrogen;

n is 0 or 1;

m is 0 or 1;

D is hydrogen, C₁₋₄alkyl, C₃₋₆cycloalkyl or fluoro;

X is —(CR⁹R¹⁰)ₜ-Q-(CR¹¹R¹²)ᵤ— where t and u are independently 0 or 1 with the proviso that t and u cannot both be 0;

Q is O, S, SO or SO₂;

R⁹, R¹⁰, R¹¹and R¹² are independently selected from hydrogen, C₁₋₄alkyl and C₃₋₆cycloalkyl; B is a group selected from aryl, heteroaryl, heterocyclyl, C₃₋₁₀cycloalkyl and C₅₋₇cycloalkenyl where each group is optionally substituted by one or more groups independently selected from nitro, trifluoromethyl, trifluoromethyloxy, halo, C₁₋₄alkyl (optionally substituted by one or more R¹³), C₂₋₄alkenyl, C₂₋₄alkynyl, C₃₋₆cycloalkyl (optionally substituted by one or more R¹³), heterocycloalkyl, heteroaryl, aryl, —OR¹³, cyano, —NR¹³R¹⁴, —CONR¹³R¹⁴, —NR¹⁶COR¹³, —SO₂NR¹³R¹⁴, —NR¹⁶SO₂R¹³, —SR¹³, —SOR⁷ and —SO₂R⁷;

R⁷ is C₁₋₆alkyl or C₃₋₆cycloalkyl

R¹³ and R¹⁴ are independently hydrogen, C₁₋₆alkyl or C₃₋₆cycloalkyl;

or R¹³ and R¹⁴ together with the nitrogen to which they are attached form a heterocyclic 4 to 7-membered ring.

Another aspect of the invention relates to compounds of formula (1) as hereinabove defined or to a pharmaceutically acceptable salt thereof.

It is to be understood that, insofar as certain of the compounds of formula (1) defined above may exist in optically active or racemic forms by virtue of one or more asymmetric carbon or sulphur atoms, the invention includes in its definition any such optically active or racemic form which possesses metalloproteinases inhibition activity and in particular TACE inhibition activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter. Compounds of formula (1) are therefore provided as enantiomers, diastereomers, geometric isomers and atropisomers.

Within the present invention it is to be understood that a compound of formula (1) or a salt thereof may exhibit the phenomenon of tautomerism and that the formulae drawings within this specification can represent only one of the possible tautomeric forms. It is to be understood that the invention encompasses any tautomeric form which has metalloproteinases inhibition activity and in particular TACE inhibition activity and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It is also to be understood that certain compounds of formula (1) and salts thereof can exist in solvated as well as unsolvated forms such as, for example, hydrated forms. It is to be understood that the invention encompasses all such solvated forms which have metalloproteinases inhibition activity and in particular TACE inhibition activity.

It is also to be understood that certain compounds of formula (1) may exhibit polymorphism, and that the invention encompasses all such forms which possess metalloproteinases inhibition activity and in particular TACE inhibition activity.

The present invention relates to the compounds of formula (1) as hereinbefore defined as well as to the salts thereof. Salts for use in pharmaceutical compositions will be pharmaceutically acceptable salts, but other salts may be useful in the production of the compounds of formula (1) and their pharmaceutically acceptable salts. Pharmaceutically acceptable salts of the invention may, for example, include acid addition salts of the compounds of formula (1) as hereinbefore defined which are sufficiently basic to form such salts. Such acid addition salts include but are not limited to hydrochloride, hydrobromide, citrate and maleate salts and salts formed with phosphoric and sulphuric acid. In addition where the compounds of formula (1) are sufficiently acidic, salts are base salts and examples include but are not limited to, an alkali metal salt for example sodium or potassium, an alkaline earth metal salt for example calcium or magnesium, or organic amine salt for example triethylamine or tris-(2-hydroxyethyl)amine.

The compounds of formula (1) may also be provided as in vivo hydrolysable esters. An in vivo hydrolysable ester of a compound of formula (1) containing carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

Suitable pharmaceutically-acceptable esters for hydroxy include inorganic esters such as phosphate esters (including phosphoramidic cyclic esters) and a-acyloxyalkyl ethers and related compounds which as a result of the in vivo hydrolysis of the ester breakdown to give the parent hydroxy group/s. Examples of a-acyloxyalkyl ethers include acetoxymethoxy and 2,2-dimethylpropionyloxymethoxy. A selection of in-vivo hydrolysable ester forming groups for hydroxy include C₁₋₁₀alkanoyl, for example formyl, acetyl; benzoyl; phenylacetyl; substituted benzoyl and phenylacetyl, C₁₋₁₀alkoxycarbonyl (to give alkyl carbonate esters), for example ethoxycarbonyl; di-(C₁₋₄)alkylcarbamoyl and N-(di-(C₁₋₄)alkylaminoethyl)-N—(C₁₋₄)alkylcarbamoyl (to give carbamates); di-(C₁₋₄)alkylaminoacetyl and carboxyacetyl. Examples of ring substituents on phenylacetyl and benzoyl include aminomethyl, (C₁₋₄)alkylaminomethyl and di-((C₁₋₄)alkyl)aminomethyl, and morpholino or piperazino linked from a ring nitrogen atom via a methylene linking group to the 3- or 4- position of the benzoyl ring. Other interesting in vivo hydrolysable esters include, for example, R^{A}C(O)O(C₁₋₆)alkyl-CO—, wherein R^{A} is for example, benzyloxy-(C₁₋₄)alkyl, or phenyl). Suitable substituents on a phenyl group in such esters include, for example, 4-(C₁₋₄)piperazino-(C₁₋₄)alkyl, piperazino-(C₁₋₄)alkyl and morpholino-(C₁₋₄)allyl.

In this specification the generic term “alkyl” includes both straight-chain and branched-chain alkyl groups. However references to individual alkyl groups such as “propyl” are specific for the straight chain version only and references to individual branched-chain alkyl groups such as tert-butyl are specific for the branched chain version only. For example, “C₁₋₃alkyl” includes methyl, ethyl, propyl and isopropyl, examples of “C₁₋₄alkyl” include the examples of “C₁₋₃alkyl”, butyl and tert-butyl and examples of “C₁₋₆alkyl” include the examples of “C₁₋₄-alkyl” and additionally pentyl, 2,3-dimethylpropyl, 3-methylbutyl and hexyl. Examples of “C₁₋₂₀alkyl” include the examples of “C₁₋₆alkyl” and other straight-chain and branched chain alkyl groups. An analogous convention applies to other generic terms, for example “C₂₋₄alkenyl” includes vinyl, allyl and 1-propenyl and examples of “C₂₋₆alkenyl” include the examples of “C₂₋₄alkenyl” and additionally 1-butenyl, 2-butenyl, 3-butenyl, 2-methylbut-2-enyl, 3-methylbut-1-enyl, 1-pentenyl, 3-pentenyl and 4-hexenyl. Examples of “C₂₋₄alkynyl” includes ethynyl, 1-propynyl and 2-propynyl and examples of “C₂₋₆alkynyl” include the examples of “C₂₋₄alkynyl” and additionally 3-butynyl, 2-pentynyl and 1-methylpent-2-ynyl.

The term “C₃₋₆cycloalkyl” includes cyclopropyl, cyclobutyl, cyclopentyl and cyclohexyl. The term “C₃₋₇cycloalkyl” includes “C₃₋₆cycloalkyl” and additionally cycloheptyl. The term “C₃₋₁₀cycloalkyl” includes “C₃₋₇cycloalkyl” and additionally cyclooctyl, cyclononyl and cyclodecyl.

“Heterocycloalkyl” is a monocyclic saturated 3- to 10-membered ring containing 1 or 2 heteroatoms selected from nitrogen, sulphur and oxygen wherein a ring nitrogen or sulphur may be oxidised to the N-oxide or S-oxide(s).

“C₅₋₇cycloalkenyl” is a monocyclic 5 to 7-membered ring containing 1, 2 or 3 double bonds. Examples are cyclopentenyl and cyclohexenyl.

The term “halo” refers to fluoro, chloro, bromo and iodo.

Examples of “C₁₋₄alkoxy” include methoxy, ethoxy, propoxy and isopropoxy. Examples of “C₁₋₆alkoxy” include the examples of “C₁₋₄alkoxy” and additionally pentyloxy, 1-ethylpropoxy and hexyloxy. Examples of “C₁₋₄alkoxycarbonyl” include methoxycarbonyl, ethoxycarbonyl, propoxycarbonyl and isopropoxycarbonyl.

Examples of “aryl” are phenyl and naphthyl.

Examples of “arylC₁₋₄alkyl” are benzyl, phenylethyl, naphthylmethyl and naphthylethyl.

“Heteroaryl” is monocyclic or bicyclic aryl ring containing 5 to 10 ring atoms of which 1, 2, 3 or 4 ring atoms are chosen from nitrogen, sulphur or oxygen where a ring nitrogen may be oxidised. Examples of heteroaryl are pyridyl, imidazolyl, quinolinyl, cinnolyl, pyrimidinyl, thienyl, pyrrolyl, pyrazolyl, thiazolyl, oxazolyl, isoxazolyl and pyrazinyl. Preferably heteroaryl is pyridyl, imidazolyl, quinolinyl, pyrimidinyl, thienyl, pyrazolyl, thiazolyl, oxazolyl and isoxazolyl. Heteroaryl is in particular pyridyl, imidazolyl, quinolinyl and pyrimidinyl.

Examples of “heteroarylC₁₋₄alkyl” are pyridylmethyl, pyridylethyl, pyrimidinylethyl, pyrimidinylpropyl, quinolinylpropyl and oxazolylmethyl.

“Heterocyclyl” is a saturated, partially saturated or unsaturated, monocyclic or bicyclic ring containing 4 to 12 atoms of which 1, 2, 3 or 4 ring atoms are chosen from nitrogen, sulphur or oxygen, which may, unless otherwise specified, be carbon or nitrogen linked, wherein a —CH₂— group can optionally be replaced by a —C(O)—; a ring nitrogen or sulphur atom may be optionally oxidised to form the N-oxide or S-oxide(s); and a —NH group may be optionally substituted by acetyl, formyl, methyl or mesyl. Examples and suitable values of the term “heterocyclyl” are piperidinyl, N-acetylpiperidinyl, N-methylpiperidinyl, piperazinyl, N-formypiperazinyl, N-mesylpiperazinyl, homopiperazinyl, azetidinyl, oxetanyl, morpholinyl, tetrahydroisoquinolinyl, tetrahydroquinolinyl, indolinyl, pyranyl, dihydro-2H-pyranyl, tetrahydrofuranyl, 2,2-dimethyl-1,3-dioxolanyl and 3,4-dimethylenedioxybenzyl. Preferred values are 3,4-dihydro-2H-pyran-5-yl, tetrahydrofuran-2-yl, 2,2-dimethyl-1,3-dioxolan-2-yl and 3,4-dimethylenedioxybenzyl.

Heterocyclic rings are rings containing 1, 2 or 3 rings atoms selected nitrogen, oxygen and sulphur. “Heterocyclic 5 to 7-membered” rings are pyrrolidinyl, piperidinyl, piperazinyl, homopiperidinyl, homopiperazinyl, thiomorpholinyl, thiopyranyl and morpholinyl. “Heterocyclic 4 to 7-membered” rings include the examples of “heterocyclic 5 to 7-membered” and additionally azetidinyl.

“Saturated heterocyclic 3 to 7-membered” rings are oxiranyl, aziridinyl, thiirane, azetidinyl, oxetanyl, thietanyl, tetrahydrothienyl, pyrrolidinyl, tetrahydrofuranyl, tetrahydro-2H-pyranyl, tetrahydro-2H-thiopyranyl and piperidinyl and a ring nitrogen may be substituted by a group selected from formyl, acetyl and mesyl.

A “carbocyclic 3 to 6-membered” ring is a saturated, partially saturated or unsaturated ring containing 3 to 6 ring carbon atoms. Examples include cyclopropyl, cyclobutyl, cyclopentyl, cyclopent-3-enyl, cyclohexyl and cyclopent-2-enyl.

Where optional substituents are chosen from “one of more” groups or substituents it is to be understood that this definition includes all substituents being chosen from one of the specified groups or the substituents being chosen from two or more of the specified groups. Preferably “one or more” means “1, 2 or 3” and this is particularly the case when the group or substituent is halo. “One or more” may also means “1 or 2”.

Compounds of the present invention have been occasionally been named with the aid of computer software (ACD/Name version 5.09).

Preferred values of Z, R¹, R³, R⁴, R⁸, n, m, D, X and B are as follows. Such values may be used where appropriate with any of the definitions, claims or embodiments defined hereinbefore or hereinafter.

In one aspect of the present invention there is provided a compound of formula (1) as depicted above wherein Z is —CONR¹⁵OH. In another aspect of the invention Z is —N(OH)CHO.

In one aspect of the invention R¹⁵ is hydrogen, methyl, ethyl or isopropyl. In another aspect R¹⁵ is hydrogen or isopropyl. In a further aspect R¹⁵ is hydrogen.

In one aspect of the invention R¹ is hydrogen or a group selected from C₁₋₆alkyl, C₂₋₆alkynyl, C₃₋₇cycloalkyl, C₅₋₇cycloalkenyl, aryl and heteroaryl where the group is optionally substituted by one or more substituents independently selected from halo, nitro, cyano, trifluoromethyl, C₁₋₄alkyl, aryl (optionally substituted by R¹⁷), heteroaryl (optionally substituted by R¹⁷), C₁₋₄alkoxycarbonyl, —OR⁵, —SR², —SOR², —SO₂R², —COR², —CO₂R⁵ and —NR¹⁶COR⁵. In another aspect R¹ is C₁₋₄alkyl, C₂₋₄alkynyl, C₃₋₆cycloalkyl, aryl, heteroaryl and C₁₋₄alkyl substituted by aryl or heteroaryl wherein any R¹ group is optionally substituted by one or more substituents independently selected from halo, cyano, nitro, C₁₋₄alkoxy, C₁₋₄alkyl, trifluoromethyl and trifluoromethoxy. In another aspect R¹ is hydrogen or a group selected from methyl, ethyl, propyl, isopropyl, tert-butyl, isobutyl, ethynyl, cyclopropyl, cyclobutyl, cyclopentyl, cyclohexyl, phenyl, naphthyl, pyridyl, thienyl, pyrimidinyl, quinolinyl, thiazolyl, oxazolyl, isoxazolyl, pyrazolyl and imidazolyl where the group is optionally substituted by one or more substituents independently selected from fluoro, chloro, bromo, nitro, cyano, trifluoromethyl, methyl, ethyl, phenyl (optionally substituted by halo or C₁₋₄alkyl), pyrimidinyl (optionally substituted by halo or C₁₋₄alkyl), C₁₋₄alkoxycarbonyl, —OR⁵, —SR², —SOR², —SO₂R², —COR², —CO₂R⁵ and —NR¹⁶COR⁵. In another aspect R¹ is selected from hydrogen, methyl, ethyl, propyl, isopropyl, tert-butyl, isobutyl, cyclopropyl, cyclobutyI, cyclopentyl, cyclohexyl, benzyloxymethyl, phenyl, benzyl, phenylethyl, phenylpropyl, (5-fluoropyrimidin-2-yl)ethyl, (5-fluoropyrimidin-2-yl)propyl, pyrimindin-2-ylethyl, pyrimidin-2-ylpropyl, naphth-2-yl, naphth-1-yl, 3,4-dichlorophenyl, 4-chlorophenyl, biphenylyl, 3-nitrophenyl, 2-trifluoromethylphenyl, 3-trifluoromethylphenyl, 4-trifluoromethylphenyl, 3-bromophenyl, 4-(methoxycarbonyl)phenyl, 4-benzyloxyphenyl, 2-fluorophenyl, 3-fluorophenyl, 4-fluorophenyl, 3-(4-chlorophenoxy)phenyl, 2-cyanophenyl, 3-cyanophenyl, 4-cyanophenyl, 2-bromothien-5-yl, 2-methylthien-5-yl, pyrimidin-2-yl, 2-methylpyrimidin-5-yl, 2-methylpyrimidin-4-yl, quinolin-4-yl, ethynyl, methoxymethyl, thiazol-2-yl, oxazol-2-yl, isoxazol-5-yl, isoxazol-3-yl, 4,4-difluorocyclohexyl, pyrimidin-2-ylmethyl, 2-pyrimidin-2-ylethyl, 3-pyrimidin-2-ylpropyl, 2,2,2-trifluoroethyl, 3-bromo-4-hydroxyphenyl, 4-fluoro-2-trifluoromethylphenyl, pyrid-2-yl, pyrid-3-yl, pyrid-4-yl, 6-methylpyrid-2-yl, 2-methylpyrid-2-yl, 5-cyanoindol-yl, pyrimidin-5-yl, imidazol-4-yl, 1H-imidazol-4-yl, pyrazol-3-yl, 1H-pyrazol-3-yl and (N-acetylamino)phenyl. In a further aspect of the invention, R¹ is methyl, ethyl, propyl, isopropyl, butyl, isobutyl, tert-butyl, cyclopropyl, cyclobutyl, cyclopentyl, phenyl (optionally substituted by 1 or 2 fluoro, chloro, trifluoromethyl, trifluoromethoxy or methyl), 3-pyrimidinylpropyl, pyridyl, imidazolyl and phenylethyl (optionally substituted on phenyl b1 or 2 fluoro, chloro, trifluoromethyl, trifluoromethoxy or methyl). In yet a further aspect R¹ is methyl, isobutyl, cyclopropyl, cyclopentyl, phenyl, 4-fluorophenyl, 2-trifluoromethylphenyl, 3-trifluoromethylphenyl, 4-fluoro-2-trifluoromethylphenyl, 3-pyrimidin-2-ylpropyl, pyrid-3-yl, imidazol-4-yl and phenylethyl. In a further aspect of the invention, R¹ is phenyl, 4-fluorophenyl, 3-pyrimidin-2-ylpropyl, 3-bromo-4-hydroxyphenyl, 3-trifluoromethylphenyl, pyrid-3-yl, methyl, imidazol-4-yl, pyrazol-3-yl and (N-acetylamino)phenyl.

In one aspect of the invention R¹⁶ is hydrogen, methyl or ethyl. In another aspect R¹⁶ is methyl or ethyl. In another aspect of the invention R¹⁶ is hydrogen.

In one aspect of the invention R¹⁷ is halo or C₁₋₄allyl. In another aspect R¹⁷ is fluoro, chloro, bromo or methyl. In another aspect of the invention R¹⁷ is fluoro or methyl.

In one aspect of the invention R² is a group selected from C₁₋₆alkyl, aryl and arylC₁₋₄alkyl where the group is optionally substituted by halo. In another aspect R² is a group selected from methyl, phenyl and benzyl where the group is optionally substituted by chloro. In one aspect of the invention R² is methyl.

In one aspect of the invention R⁵is hydrogen or a group selected from C₁₋₆alkyl, aryl and arylC₁₋₄alkyl where the group is optionally substituted by halo. In another aspect R⁵ is hydrogen or a group selected from methyl, phenyl and benzyl where the group is optionally substituted by chloro.

In one aspect of the invention R⁶ is hydrogen, methyl, ethyl, propyl or isopropyl.

In one aspect of the invention R⁸ is hydrogen, methyl, ethyl, propyl or isopropyl. In another aspect R⁸ is hydrogen.

In one aspect of the invention R³ is hydrogen, methyl, ethyl or phenyl. In another aspect R³ is hydrogen.

In one aspect of the invention R⁴ is hydrogen, methyl, ethyl or phenyl. In another aspect R⁴ is hydrogen.

In one aspect of the invention n is 0. In another aspect n is 1.

In one aspect of the invention m is 0. In another aspect of the invention m is 1.

In one aspect of the invention D is hydrogen, methyl or fluoro. In another aspect D is hydrogen.

In one aspect of the invention X is —CR⁹R¹⁰-Q-, -Q-CR¹¹R¹²— or —CR⁹R¹⁰-Q-CR¹¹R¹²—. In another aspect of the invention X is —(CH₂)-Q-, -Q-(CH₂)— or —(CH₂)-Q-(CH₂)— or —(CHMe)-Q-. In a further aspect of the invention X is —(CH₂)—O—, —O—(CH₂)—, —(CH₂)—O—(CH₂)— or —(CHMe)—O—. In yet another aspect X is —(CH₂)—O—.

In one aspect of the invention Q is 0.

In one aspect of the invention t is 1. In another aspect t is 0, provided that u is not 0.

In one aspect of the invention u is 1. In another aspect u is 0, provided that t is not 0.

In one aspect of the invention R⁹ is hydrogen or methyl.

In one aspect of the invention R¹⁰ is hydrogen.

In one aspect of the invention R¹¹ is hydrogen.

In one aspect of the invention R¹² is hydrogen.

In one aspect of the invention B is a group selected from aryl, heteroaryl, heterocyclyl, C₃₋₁₀cycloalkyl and C₅₋₇cycloalkenyl where each group is optionally substituted by one or more groups independently selected from nitro, trifluoromethyl, halo, C₁₋₄alkyl, heteroaryl, —OR¹³, cyano, —NR¹³R¹⁴, —CONR¹³R¹⁴ and —NR¹⁶COR¹³. In another aspect B is aryl, heteroaryl or C₃₋₆cycloalkyl optionally substituted by 1, 2 or 3 groups independently selected from C₁₋₄alkyl, halo, cyano, nitro, C₁₋₄alkoxy and trifluoromethyl. In another aspect B is phenyl, naphthyl, pyridyl, quinolinyl, isoquinolinyl, thieno[2,3-b]pyridyl, thieno[3,2-b]pyridyl, 1,8-naphthyridinyl, cyclohexyl, 3,4-rnethylenedioxybenzyl where each group is optionally substituted by one or more groups independently selected from nitro, trifluoromethyl, trifluoromethyloxy, halo, C₁₋₄alkyl (optionally substituted by one or more R¹³), C₂₋₄alkynyl, C₃₋₆cycloalkyl (optionally substituted by one or more R¹³), heteroaryl, —OR¹³, cyano, —NR¹³R¹⁴, —CONR¹³R¹⁴, —NR¹⁶COR¹³, —SO₂NR¹³R¹⁴, —NR¹⁶SO₂R¹³, —SR¹³, —SOR⁷ and —SO₂R. In another aspect B is phenyl, naphthyl, pyridyl, quinolinyl, isoquinolinyl, thieno[2,3-b]pyridyl, thieno[3,2-b]pyridyl, 1,8-naphthyridinyl, cyclohexyl, 3,4-methylenedioxybenzyl where each group is optionally substituted by one or more groups independently selected from trifluoromethyl, fluoro, chloro, bromo, methyl, isopropyl or cyano. In another aspect B is phenyl, quinolinyl, pyxidyl and cyclohexyl optionally substituted by 1, 2 or 3 halo, methyl, isopropyl, methoxy or trifluoromethyl. In another aspect B is quinolin-4-yl, naphthyl, 2-methylquinolin-4-yl, 3-methylnaphthyl, 7-methylquinolin-5-yl, 6-methylquinolin-8-yl, 7-methylisoquinolin-5-yl, 6-methylthieno[2,3-b]pyridyl, 5-methylthieno[3,2-b]pyridyl, 2-methyl-1,8-naphthyridinyl, 2-trifluoromethylquinolin-4-yl, 2-ethynylquinolin-4-yl, 7-chloroquinolin-5-yl, 7-fluoro-2-methylquinolinyl, 2-methyl-N-oxoquinolin-4-yl, 3-methylisoquinolin-1-yl, 5-fluoro-2-methylquinolin-4-yl, 2,6-dimethylpyrid-4-yl, 2,5-dimethylpyridin-4-yl, 2,5-dimethylphenyl, 3-methoxyphenyl, 2,5-difluorophenyl, 3,5-difluorophenyl, pyrid-2-yl, pyrid-3-yl, pyridyl, 2,6-difluoro-3-methylphenyl, 2-chloro-6-fluorophenyl, 3-fluoro-6-methylphenyl, phenyl, 2-methylphenyl, 3-chlorophenyl, 2-bromophenyl, 2-fluorophenyl, 2,6-difluorophenyl, 3-fluorophenyl, 4-trifluoromethylphenyl, 2-chlorophenyl, 3,4-dichlorophenyl, 4-chlorophenyl, cyclohexyl, 4-bromophenyl, 2-cyanophenyl, 4-fluorophenyl, 2-fluoro-3-methylphenyl, 4-methylphenyl, 2,4-dichlorophenyl, 2,6-dichlorophenyl, 2,4,6-trimethylphenyl, 3-methylphenyl, 3,4-dimethylphenyl, 4-methoxyphenyl, 3,5-dimethylphenyl, 4-prop-2-ylphenyl, 3-chloro-4-methylphenyl, 3,4-methylenedioxybenzyl, 5-fluoro-2-methylpyridinyl, 2,4-dimethylphenyl or 1-methylquinolinyl. In a further aspect, B is phenyl, naphthyl, 2-bromophenyl, 3-bromophenyl, 4-bromophenyl, 2-chlorophenyl, 3-chlorophenyl, 4-chlorophenyl, 2-fluorophenyl, 3-fluorophenyl, 4-fluorophenyl, 2-methylphenyl, 3-methylphenyl, 4-methylphenyl, 4-trifluoromethylphenyl, 3-methoxyphenyl, 4-methoxyphenyl, 4-isopropylphenyl, 2,4-dichlorophenyl, 2,6-dichlorophenyl, 3,4-dichlorophenyl, 2,5-difluorophenyl, 3,5-difluorophenyl, 2,6-difluorophenyl, 2-chloro-6-fluorophenyl, 2,5-dimethylphenyl, 3,4-dimethylphenyl, 3,5-dimethylphenyl, 2,4,6-trimethylphenyl, 5-fluoro-2-methylphenyl, 2-fluoro-3-methylphenyl, 2,6-difluoro-3-methylphenyl, 3-chloro-4-methylphenyl, cyclohexyl, pyrid-2-yl, pyrid-3-yl, pyrid-4-yl, 2,6-dimethylpyrid-4-yl and 2-methylquinolin-4-yl. In yet a further aspect B is 2,5-difluorophenyl, 2,5-dimethylphenyl, 2-cyanophenyl, 2-methylquinolin-4yl or 2,5-dimethylpyrid-4-yl. In another aspect B is 2,5-dimethylphenyl or 2-methylquinolin-4-yl.

In one aspect of the invention R⁷ is C₁₋₄alkyl. In another aspect R⁷ is methyl, ethyl, propyl or isopropyl.

In one aspect of the invention R¹³ is hydrogen or C₁₋₄alkyl. In another aspect R¹³ is methyl.

In one aspect of the invention R¹⁴ is hydrogen or C₁₋₄alkyl. In another aspect R¹⁴ is hydrogen or methyl.

In a further aspect R¹³ and R¹⁴ together with the nitrogen to which they are attached form a heterocyclic 5 to 7-membered ring.

A preferred class of compound is of formula (1) wherein;

Z is —N(OH)CHO;

R¹ is hydrogen or a group selected from C₁₋₆alkyl, C₂₋₆alkynyl, C₃₋₇cycloalkyl, C₅₋₇cycloalkenyl, aryl and heteroaryl where the group is optionally substituted by one or more substituents independently selected from halo, nitro, cyano, trifluoromethyl, C₁₋₄alkyl, aryl (optionally substituted by R¹⁷), heteroaryl (optionally substituted by R¹⁷), C₁₋₄alkoxycarbonyl, —OR⁵, —SR², —SOR², —SO₂R², —COR², —CO₂R⁵ and —NR¹⁶COR⁵;

R¹⁶ is hydrogen, methyl or ethyl;

R¹⁷ is halo or C₁₋₄alkyl;

R² is a group selected from C₁₋₆alkyl, aryl and arylC₁₋₄alkyl where the group is optionally substituted by halo;

R⁵ is hydrogen or a group selected from C₁₋₆alkyl, aryl and arylC₁₋₄alkyl where the group is optionally substituted by halo;

R⁶ is hydrogen, methyl, ethyl, propyl or isopropyl;

R⁸ is hydrogen, methyl, ethyl, propyl or isopropyl;

R³ is hydrogen;

R⁴ is hydrogen;

n is 0;

m is 1;

D is hydrogen, methyl or fluoro;

X is —(CH₂)—O—, —O—(CH₂)— or —(CH₂)—O—(CH₂)— or —(CHMe)—O—;

B is a group selected from aryl, heteroaryl, heterocyclyl, C₃₋₁₀cycloalkyl and C₅₋₇cycloalkenyl where each group is optionally substituted by one or more groups independently selected from nitro, trifluoromethyl, halo, C₁₋₄alkyl, heteroaryl, —OR¹³, cyano, —NR¹³R¹⁴, —CONR¹³R¹⁴ and —NR¹⁶COR¹³;

R⁷ is C₁₋₄alkyl;

R¹³ is hydrogen or C₁₋₄alkyl; and

R¹⁴ is hydrogen or C₁₋₄alkyl.

Another preferred class of compounds are of formula (1) wherein:

Z is —CONR¹⁵H;

R¹⁵ is hydrogen, methyl, ethyl or isopropyl.

R¹ is hydrogen or a group selected from C₁₋₆alkyl, C₂₋₆alkynyl, C₃₋₇cycloalkyl, C₅₋₇cycloalkenyl, aryl and heteroaryl where the group is optionally substituted by one or more substituents independently selected from halo, nitro, cyano, trifluoromethyl, C₁₋₄alkyl, aryl (optionally substituted by R¹⁷), heteroaryl (optionally substituted by R¹⁷), C₁₋₄alkoxycarbonyl, —OR⁵, —SR², —SOR², —SO₂R², —COR², —CO₂R⁵ and —NR¹⁶COR⁵;

R¹⁵ is hydrogen, methyl, ethyl or isopropyl;

R¹⁶ is hydrogen;

R¹⁷ is halo or C₁₋₄alkyl;

R² is a group selected from C₁₋₆alkyl, aryl and arylC₁₋₄alkyl where the group is optionally substituted by halo;

R⁵ is hydrogen or a group selected from C₁₋₆alkyl, aryl and arylC₁₋₄alkyl where the group is optionally substituted by halo;

R⁶ is hydrogen, methyl, ethyl, propyl or isopropyl;

R⁸ is hydrogen, methyl, ethyl, propyl or isopropyl;

R³ is hydrogen;

R⁴ is hydrogen;

n is 0;

m is 1;

D is hydrogen, methyl or fluoro;

X is —(CH₂)—O—, —O—(CH₂)— or —(CH₂)—O—(CH₂)— or —(CHMe)—O—;

B is a group selected from aryl, heteroaryl, heterocyclyl, C₃₋₁₀cycloalkyl and C₅₋₇cycloalkenyl where each group is optionally substituted by one or more groups independently selected from nitro, trifluoromethyl, halo, C₁₋₄alkyl, heteroaryl, —OR¹³, cyano, —NR¹³R¹⁴, —CONR¹³R¹⁴ and —NR¹⁶COR¹³;

R⁷ is C₁₋₄alkyl;

R¹³ is hydrogen or C₁₋₄alkyl; and

R¹⁴ is hydrogen or C₁₋₄alkyl.

Another preferred class of compounds are of formula (1) wherein:

Z is —N(OH)CHO or —CONR¹⁵OH;

R¹⁵ is hydrogen, methyl, ethyl or isopropyl;

R¹ is C₁₋₄alkyl, C₂₋₄alkynyl, C₃₋₆cycloalkyl, aryl, heteroaryl and C₁₋₄alkyl substituted by aryl or heteroaryl wherein any R¹ group is optionally substituted by one or more substituents independently selected from halo, cyano, nitro, C₁₋₄alkoxy, C₁₋₄alkyl, trifluoromethyl and trifluoromethoxy;

R⁸ is hydrogen, methyl, ethyl, propyl or isopropyl;

R³ is hydrogen;

R⁴ is hydrogen;

n is 0;

m is 1;

D is hydrogen, methyl or fluoro;

X is —(CH₂)—O—, —O—(CH₂)— or —(CH₂)—O—(CH₂)— or —(CHMe)—O—; and

B is aryl, heteroaryl or C₃₋₆cycloalkyl optionally substituted by 1, 2 or 3 groups independently selected from C₁₋₄alkyl, halo, cyano, nitro, C₁₋₄alkoxy and trifluoromethyl.

A further preferred class of compounds are of formula (1) wherein:

Z is —N(OH)CHO or —CONR¹⁵OH;

R¹⁵ is hydrogen or isopropyl;

R¹ is methyl, ethyl, propyl, isopropyl, butyl, isobutyl, tert-butyl, cyclopropyl, cyclobutyl, cyclopentyl, phenyl (optionally substituted by 1, 2 or 3 fluoro, chloro, trifluoromethyl, trifluoromethoxy or methyl), 3-pyrimidinylpropyl, pyridyl, imidazolyl and phenylethyl (optionally substituted on phenyl by 1, 2 or 3 fluoro, chloro, trifluoromethyl, trifluoromethoxy and methyl);

R⁸ is hydrogen;

R³ is hydrogen;

R⁴ is hydrogen;

n is 0;

m is 1;

D is hydrogen, methyl or fluoro;

X is —(CH₂)—O—, —O—(CH₂)— or —(CH₂)—O—(CH₂)— or —(CHMe)—O—; and

B is phenyl, quinolinyl, pyridyl and cyclohexyl optionally substituted by 1, 2 or 3 halo, methyl, isopropyl, methoxy or trifluoromethyl.

In another aspect of the invention, preferred compounds of the invention are any one of:
1-[({4-[(2,5-dimethylbenzyl)oxy]piperidin-1-yl}sulphonyl)methyl]-3-phenylpropyl(hydroxy)formamide;
2-({4-[(3-methoxybenzyl)oxy]piperidin-1-yl}sulphonyl)-1-phenylethyl(hydroxy)formamide;
2-({4-[(2,5-difluorobenzyl)oxy]piperidin-1-yl}sulphonyl)-1-phenylethyl(hydroxy)formamide;
2-({4-[(3,5-difluorobenzyl)oxy]piperidin-1-yl}sulphonyl)-1-phenylethyl(hydroxy)formamide;
1-phenyl-2-{[4-(pyridin-2-ylmethoxy)piperidin-1-yl]sulphonyl}ethyl(hydroxy)formamide;
1-phenyl-2-{[4-(pyridin-3-ylmethoxy)piperidin-1-yl]sulphonyl}ethyl(hydroxy)formamide;
2-({4-[(2,6-difluoro-3-methylbenzyl)oxy]piperidin-1-yl}sulphonyl)-1-phenylethyl(hydroxy)formamide;
2-({4-[(2-chloro-6-fluorobenzyl)oxy]piperidin-1-yl}sulphonyl)-1-phenylethyl(hydroxy)formamide;
2-({4-[(5-fluoro-2-methylbenzyl)oxy]piperidin-1-yl}sulphonyl)-1-phenylethyl(hydroxy)formamide;
2-{[4-(benzyloxy)piperidin-1-yl]sulphonyl}-1-phenylethyl(hydroxy)formamide;
2-({4-[(2-methylbenzyl)oxy]piperidin-1-yl}sulphonyl)-1-phenylethyl(hydroxy)formamide;
2-({4-[(3-chlorobenzyl)oxy]piperidin-1-yl}sulphonyl)-1-phenylethyl(hydroxy)formamide;
2-({4-[(2-bromobenzyl)oxy]piperidin-1-yl}sulphonyl)-1-phenylethyl(hydroxy)formamide;
2-({4-[(2-fluorobenzyl)oxy]piperidin-1-yl}sulphonyl)-1-phenylethyl(hydroxy)formamide;
2-({4-[(2,6-difluorobenzyl)oxy]piperidin-1-yl}sulphonyl)-1-phenylethyl(hydroxy)formamide;
2-({4-[(3-fluorobenzyl)oxy]piperidin-1-yl}sulphonyl)-1-phenylethyl(hydroxy)formamide;
2-({4-[(4-trifluoromethylbenzyl)oxy]piperidin-1-yl}sulphonyl)-1-phenylethyl(hydroxy)formamide;
2-({4-[(2-chlorobenzyl)oxy]piperidin-1-yl}sulphonyl)-1-phenylethyl(hydroxy)formamide;
2-({4-[(3,4-dichlorobenzyl)oxy]piperidin-1-yl}sulphonyl)-1-phenylethyl(hydroxy)formamide;
2-({4-[(4-chlorobenzyl)oxy]piperidin-1-yl}sulphonyl)-1-phenylethyl(hydroxy)formamide;
2-{[4-(cyclohexylmethoxy)piperidin-1-yl]sulphonyl}-1-phenylethyl(hydroxy)formamide;
2-{[4-(2-naphthylmethoxy)piperidin-1-yl]sulphonyl}-1-phenylethyl(hydroxy)formamide;
2-({4-[(4-bromobenzyl)oxy]piperidin-1-yl}sulphonyl)-1-phenylethyl(hydroxy)formamide;
2-({4-[(2,5-dimethylphenoxy)methyl]piperidin-1-yl}sulphonyl)-1-(4-fluorophenyl)ethyl(hydroxy)formamide;
1-benzyl-2-({4-[(2,5-dimethylbenzyl)oxy]piperidin-1-yl}sulphonyl)ethyl(hydroxy)formamide;
2-({4-[(2,5-dimethylbenzyl)oxy]piperidin-1-yl}sulphonyl)-1-(4-fluorophenyl)ethyl(hydroxy)formamide;
2-({4-[(4-fluorobenzyl)oxy]piperidin-1-yl}sulphonyl)-1-phenylethyl(hydroxy)formamide;
2-({4-[(2,5-dimethylbenzyl)oxy]piperidin-1-yl}sulphonyl)-1-phenylethyl(hydroxy)formamide;
1-phenyl-2-{[4-(pyridin-4-ylmethoxy)piperidin-1-yl]sulphonyl}ethyl(hydroxy)formamide;
2-({4-[(2-fluoro-3-methylbenzyl)oxy]piperidin-1-yl}sulphonyl)-1-phenylethyl(hydroxy)formamide;
1-({[4-(benzyloxy)piperidin-1-yl]sulphonyl}methyl)-4pyrimidin-2-ylbutyl(hydroxy)formamide;
2-({4-[(2,5-dimethylbenzyl)oxy]piperidin-1-yl}sulphonyl)-1-methylethyl(hydroxy)formamide;
1-(3-bromo-4hydroxyphenyl)-2-({4-[(2,5-dimethylbenzyl)oxy]piperidin-1-yl}sulphonyl)ethyl(hydroxy)formamide;
2-({4-[(2,5-dimethylbenzyl)oxy]piperidin-1-yl}sulphonyl)-1-[4-fluoro-2-(trifluoromethyl)phenyl]ethyl(hydroxy)formamide;
2-({4-[(2,5-dimethylbenzyl)oxy]piperidin-1-yl}sulphonyl)-1-[2-(trifluoromethyl)phenyl]ethyl(hydroxy)formamide;
2-({4-[(2,5-dimethylbenzyl)oxy]piperidin-1-yl}sulphonyl)-1-[3-(trifluoromethyl)phenyl]ethyl(hydroxy)formamide;
2-({4-[(2,5-dimethylbenzyl)oxy]piperidin-1-yl}sulphonyl)-1-pyridin-3-ylethyl(hydroxy)formamide;
1-[({4-[(2,5-dimethylbenzyl)oxy]piperidin-1-yl}sulphonyl)methyl]-4-pyrimidin-2-ylbutyl(hydroxy)formamide;
2-({4-[(2-methylquinolin-4-yl)methoxy]piperidin-1-yl}sulphonyl)-1-phenylethyl(hydroxy)formamide;
1-methyl-2-({4-[(2-methylquinolin-4yl)methoxy]piperidin-1-yl}sulphonyl)ethyl(hydroxy)formamide;
1-[({4-[(2-methylquinolin-4-yl)methoxy]piperidin-1-yl}sulphonyl)methyl]-4-pyrimidin-2-ylbutyl(hydroxy)formamide;
1-{[(4-[(2,5-dimethylbenzyl)oxy]methyl}piperidin-1-yl)sulphonyl]methyl}-4-pyrimidin-2-ylbutyl(hydroxy)formamide;
2-({4-[(2-methylquinolin-4-yl)methoxy]piperidin-1-yl}sulphonyl)-1-pyridin-3-ylethyl(hydroxy)formamide;
1-(1H-imidazol-4-yl)-2-({4-[(2-methylquinolin-4-yl)methoxy]piperidin-1-yl}sulphonyl)ethyl(hydroxy)formamide;
2-({4-[(2-methylquinolin-4-yl)methoxy]piperidin-1-yl}sulphonyl)-1-(1H-pyrazol-3-yl)ethyl(hydroxy)formamide;
2-({4-[(2-methylquinolin-4-yl)methoxy]piperidin-1-yl}sulphonyl)-1-(4-acetamidophenyl)ethyl(hydroxy)formamide;
3-({4-[(2,5-dimethylbenzyl)oxy]piperidin-1-yl}sulphonyl)-N-hydroxy-2-phenylpropanamide;
1-[({4-[(2,6-dimethylpyridin-4-yl)methoxy]piperidin-1-yl}sulphonyl)methyl]-4-pyrimidin-2-ylbutyl(hydroxy)formamide;
2-{[4-(1-phenylethoxy)piperidin-1-yl]sulphonyl}-1-pyridin-3-ylethyl(hydroxy)formamide;
2-({4-[(2-methylbenzyl)oxy]piperidin-1-yl}sulphonyl)-1-pyridin-3-ylethyl(hydroxy)formamide;
2-({4-[(4-methylbenzyl)oxy]piperidin-1-yl}sulphonyl)-1-pyridin-3-ylethyl(hydroxy)formamide;
2-({4-[(2-fluorobenzyl)oxy]piperidin-1-yl}sulphonyl)-1-pyridin-3-ylethyl(hydroxy)formamide;
2-({4-[(3-fluorobenzyl)oxy]piperidin-1-yl}sulphonyl)-1-pyridin-3-ylethyl(hydroxy)formamide;
2-({4-[(2-chlorobenzyl)oxy]piperidin-1-yl}sulphonyl)-1-pyridin-3-ylethyl(hydroxy)formamide;
2-({4-[(2,4-dichlorobenzyl)oxy]piperidin-1-yl}sulphonyl)-1-pyridin-3-ylethyl(hydroxy)formamide;
2-({4-[(2-chloro-5-fluorobenzyl)oxy]piperidin-1-yl}sulphonyl)-1-pyridin-3-ylethyl(hydroxy)formamide;
2-({4-[(2,6-dichlorobenzyl)oxy]piperidin-1-yl}sulphonyl)-1-pyridin-3-ylethyl(hydroxy)formamide;
2-({4-[(2,4,6-trimethylbenzyl)oxy]piperidin-1-yl}sulphonyl)-1-pyridin-3-ylethyl(hydroxy)formamide;
2-({4-[(3-chlorobenzyl)oxy]piperidin-1-yl}sulphonyl)-1-pyridin-3-ylethyl(hydroxy)formamide;
2-({4-[(3,4-dichlorobenzyl)oxy]piperidin-1-yl}sulphonyl)-1-pyridin-3-ylethyl(hydroxy)formamide;
2-({4-[(3-methoxybenzyl)oxy]piperidin-1-yl}sulphonyl)-1-pyridin-3-ylethyl(hydroxy)formamide;
2-({4-[(3-methybenzyl)oxy]piperidin-1-yl}sulphonyl)-1-pyridin-3-ylethyl(hydroxy)formamide;
2-([4-[(3,4-dimethybenzyl)oxy]piperidin-1-yl}sulphonyl)-1-pyridin-3-ylethyl(hydroxy)formamide;
2-({4-[(4-chlorobenzyl)oxy]piperidin-1-yl}sulphonyl)-1-pyridin-3-ylethyl(hydroxy)formamide;
2-({4-[(4-methoxybenzyl)oxy]piperidin-1-yl}sulphonyl)-1-pyridin-3-ylethyl(hydroxy)formamide;
2-({4-[(3,5-dimethybenzyl)oxy]piperidin-1-yl}sulphonyl)-1-pyridin-3-ylethyl(hydroxy)formamide;
2-({4-[(4-isopropylbenzyl)oxy]piperidin-1-yl}sulphonyl)-1-pyridin-3-ylethyl(hydroxy)formamide;
2-({4-[(3chloro-4-methylbenzyl)oxy]piperidin-1-yl}sulphonyl)-1-pyridin-3-ylethyl(hydroxy)formamide;
2-{[4-(1,3-benzodioxol-5-ylmethoxy)piperidin-1-yl]sulphonyl}-1-pyridin-3-ylethyl(hydroxy)formamide;
1-[({4-[(2,5-dimethylpyridin-4-yl)methoxy]piperidin-1-yl}sulphonyl)methyl]-4-pyrimidin-2-ylbutyl(hydroxy)formamide;
3-({4-[(2,5-dimethylbenzyl)oxy]piperidin-1-yl}sulphonyl)-1-phenylpropyl(hydroxy)formamide;
1-{[(4-{[(2-methylquinolin-4-yl)methoxy]methyl}piperidin-1-yl)sulphonyl]methyl}-4-pyrimidin-2-ylbutyl(hydroxy)formamide;
1-[({4-[(2-methylquinolin-4-yl)methoxy]piperidin-1-yl}sulphonyl)methyl]propyl(hydroxy)formamide;
2-methyl-1-[({4-[(2-methylquinolin-4-yl)methoxy]piperidin-1-yl}sulphonyl)methyl]propyl(hydroxy)formamide;
3-methyl-1-[({4-[(2-methylquinolin-4-yl)methoxy]piperidin-1-yl}sulphonyl)methyl]butyl(hydroxy)formamide;
1-cyclopropyl-2-({4-[(2-methylquinolin-4-yl)methoxy]piperidin-1-yl}sulphonyl)ethyl(hydroxy)formamide;
1-(3-fluorophenyl)-2-({4-[(2-methylquinolin-4-yl)methoxy]piperidin-1-yl}sulphonyl)ethyl(hydroxy)formamide;
1-(4-fluorophenyl)-2-({4-[(2-methylquinolin-4-yl)methoxy]piperidin-1-yl}sulphonyl)ethyl(hydroxy)formamide;
1-(3-trifluoromethylphenyl)-2-({4-[(2-methylquinolin-4-yl)methoxy]piperidin-1-yl}sulphonyl)ethyl(hydroxy)formamide; and
1-(4-trifluoromethylphenyl)-2-({4-[(2-methylquinolin-4-yl)methoxy]piperidin-1-yl}sulphonyl)ethyl(hydroxy)formamide.

In a further aspect, preferred compounds are any one of:
(R/S)-1-[({4-[(2-methylquinolin-4-yl)methyloxy]piperidin-1-yl}sulphonyl)methyl]-4-pyrimidin-2-ylbutyl(hydroxy)formamide;
(R/S)-1-methyl-2-({4-[(2-methylquinolin-4-yl)methoxy]piperidin-1-yl}sulphonyl)ethyl(hydroxy)formamide;
(R/S)-1-pyrid-3-yl-2-({4-[(2-methylquinolin-4-yl)methoxy]piperidin-1-yl}sulphonyl)ethyl(hydroxy)formamide;
(R/S)-1-(1H-imidazol-4-yl)-2-({4-[(2-methylquinolin-4-yl)methoxy]piperidin-1-yl}sulphonyl)ethyl(hydroxy)formamide;
(R/S)-2-({4-[(2,5-dimethylbenzyl)oxy]piperidin-1-yl}sulphonyl)-1-pyrid-3-ylethyl(hydroxy)formamide;
(R/S)-[1-({[4-(2,5-dimethylbenzyloxy]piperidin-1-yl]sulphonyl}methyl)-3-phenylpropyl]hydroxyformamide;
(R/S)-2-({4-[(2,5-dimethylbenzyl)oxy]piperidin-1-yl}sulphonyl)-1-[4-fluoro-2-(trifluoromethyl)phenyl]ethyl(hydroxy)formamide;
(R/S)-2-({4-[(2,5-dimethylbenzyl)oxy]piperidin-1-yl}sulphonyl)-1-[2-(trifluoromethyl)phenyl]ethyl(hydroxy)formamide;
(R/S)-2-({4-[(2,5-dimethylbenzyl)oxy]piperidin-1-yl}sulphonyl)-1-[3-(trifluoromethyl)phenyl]ethyl(hydroxy)formamide;
(R/S)-2-({4-[(2,5-dimethylphenoxy)methyl]piperidin-1-yl}sulphonyl)-1-(4-fluorophenyl)ethyl(hydroxy)formamide;
(R/S)-1-{[(4-{[(2,5-dimethylbenzyl)oxy]methyl}piperidin-1-yl)sulphonyl]methyl}-4-pyrimidin-2-ylbutyl(hydroxy)formamide
(R/S)-2-methyl-3-({4-[(2-methylquinolin-4-yl)methoxy]piperidin-1-yl}sulphonyl)propionic hydroxamic acid
(R/S)-2-({4-[(2,5-difluorobenzyl)oxy]piperidin-1-yl}sulphonyl)-1-phenylethyl(hydroxy)formamide
(R/S)-hydroxy(1-phenyl-2-{[4-(pyridin-2-ylmethoxy)piperidin-1-yl]sulphonyl}ethyl)formamide;
(R/S)-hydroxy(1-phenyl-2-{[4-(pyridin-3-ylmethoxy)piperidin-1-yl]sulphonyl}ethyl)formamide;
(R/S)-2-({4-[(2,6-difluoro-3-methylbenzyl)oxy]piperidin-1-yl}sulphonyl)-1-phenylethyl(hydroxy)formamide;
(R/S)-2-({4-[(2-chloro-6-fluorobenzyl)oxy]piperidin-1-yl}sulphonyl)-1-phenylethyl(hydroxy)formamide;
(R/S)-2-({4-[(5-fluoro-2-methylbenzyl)oxy]piperidin-1-yl}sulphonyl)-1-phenylethyl(hydroxy)formamide;
(R/S)-2-{[4-(benzyloxy)piperidin-1-yl]sulphonyl}-1-phenylethyl(hydroxy)formamide;
(R/S)-hydroxy[2-({4-[(2-methylbenzyl)oxy]piperidin-1-yl}sulphonyl)-1-phenylethyl]formamide;
(R/S)-2-({4-[(3-chlorobenzyl)oxy]piperidin-1-yl}sulphonyl)-1-phenylethyl(hydroxy)formamide;
(R/S)-2-({4-[(2-bromobenzyl)oxy]piperidin-1-yl}sulphonyl)-1-phenylethyl(hydroxy)formamide;
(R/S)-2-({4-[(2-fluorobenzyl)oxy]piperidin-1-yl}sulphonyl)-1-phenylethyl(hydroxy)formamide;
(R/S)-2-({4-[(2,6-difluorobenzyl)oxy]piperidin-1-yl}sulphonyl)-1-phenylethyl(hydroxy)formamide;
(R/S)-2-({4-[(3-fluorobenzyl)oxy]piperidin-1-yl}sulphonyl)-1-phenylethyl(hydroxy)formamide;
(R/S)-hydroxy{1-phenyl-2-[(4-{[4-(trifluoromethyl)benzyl]oxy}piperidin-1-yl)sulphonyl]ethyl}formamide;
(R/S)-2-{[4-(cyclohexylmethoxy)piperidin-1-yl]sulphonyl}-1-phenylethyl(hydroxy)formamide;
(R/S)-2-({4-[(4-bromobenzyl)oxy]piperidin-1-yl}sulphonyl)-1-phenylethyl(hydroxy)formamide;
(R/S)-2-({4-[(4-fluorobenzyl)oxy]piperidin-1-yl}sulphonyl)-1-phenylethyl(hydroxy)formamide;
(R/S)-2-({4-[(2,5-dimethylbenzyl)oxy]piperidin-1-yl}sulphonyl)-1-phenylethyl(hydroxy)formamide;
(R/S)-2-({4-[(2-fluoro-3-methylbenzyl)oxy]piperidin-1-yl}sulphonyl)-1-phenylethyl(hydroxy)formamide;
(R/S)-hydroxy[2-({4-[(2-methylbenzyl)oxy]piperidin-1-yl}sulphonyl)-1-pyridin-3-ylethyl]formamide;
(R/S)-hydroxy[2-({4-[(4-methylbenzyl)oxy]piperidin-1-yl}sulphonyl)-1-pyridin-3-ylethyl]formamide;
(R/S)-2-({4-[(2-fluorobenzyl)oxy]piperidin-1-yl}sulphonyl)-1-pyridin-3-ylethyl(hydroxy)formamide;
(R/S)-2-({4-[(2-chlorobenzyl)oxy]piperidin-1-yl}sulphonyl)-1-pyridin-3-ylethyl(hydroxy)formamide;
(R/S)-2-({4-[(2,4-dichlorobenzyl)oxy]piperidin-1-yl}sulphonyl)-1-pyridin-3-ylethyl(hydroxy)formamide;
(R/S)-2-({4-[(2,6-dichlorobenzyl)oxy]piperidin-1-yl}sulphonyl)-1-pyridin-3-ylethyl(hydroxy)formamide;
(R/S)-hydroxy(2-{[4-(methylbenzyl)oxy]piperidin-1-yl]sulphonyl}-1-pyridin-3-ylethyl)formamide;
(R/S)-2-({4-[(3,4-dichlorobenzyl)oxy]piperidin-1-yl}sulphonyl)-1-pyridin-3-ylethyl(hydroxy)formamide;
(R/S)-hydroxy[2-({4-[(3-methoxybenzyl)oxy]piperidin-1-yl}sulphonyl)-1-pyridin-3-ylethyl]formamide;
(R/S)-hydroxy[2-({4-[(3-methylbenzyl)oxy]piperidin-1-yl}sulphonyl)-1-pyridin-3-ylethyl]formamide;
(R/S)-2-({4-[(3,4-dimethylbenzyl)oxy]piperidin-1-yl}sulphonyl)-1-pyridin-3-ylethyl(hydroxy)formamide;
(R/S)-hydroxy[2-({4-[(4-methoxybenzyl)oxy]piperidin-1-yl}sulphonyl)-1-pyridin-3-ylethyl]formamide;
(R/S)-hydroxy[2-({4-[(4-isopropylbenzyl)oxy]piperidin-1-yl}sulphonyl)-1-pyridin-3-ylethyl]formamide;
(R/S)-2-({4-[(3-chloro-4-methylbenzyl)oxy]piperidin-1-yl}sulphonyl)-1-pyridin-3-ylethyl(hydroxy)formamide;
(R/S)—N-hydroxy-N-isopropyl-2-methyl-3-({4-[(2-methylquinolin-4-yl)methoxy]piperidin-1-yl}sulphonyl)propanamide;
hydroxy{(1R)-1-[({4-[(2-methylquinolin-4-yl)methoxy]piperidin-1-yl}sulphonyl)methyl]-4-pyrimidin-2-ylbutyl}formamide;
hydroxy{(1S)-1-[({4-[(2-methylquinolin-4-yl)methoxy]piperidin-1-yl}sulphonyl)methyl]-4-pyrimidin-2-ylbutyl}formamide;
(2R)—N-hydroxy-2-methyl-3-({4-[(2-methylquinolin-4-yl)methoxy]piperidin-1-yl}sulphonyl)propanamide
(R/S)-2-cyclopentyl-N-hydroxy-3-({4-[(2-methylquinolin-4-yl)methoxy]piperidin-1-yl}sulphonyl)propanamide;
(2S)-2-cyclopentyl-N-hydroxy-3-({4-[(2-methylquinolin-4-yl)methoxy]piperidin-1-yl}sulphonyl)propanamide;
(2R)-2-cyclopentyl-N-hydroxy-3-({4-[(2-methylquinolin-4-yl)methoxy]piperidin-1-yl}sulphonyl)propanamide;
(2S)—N-hydroxy-4-methyl-2-[({4-[(2-methylquinolin-4-yl)methoxy]piperidin-1-yl}sulphonyl)methyl]pentanamide;
(2R)—N-hydroxy-4-methyl-2-[({4-[(2-methylquinolin-4-yl)methoxy]piperidin-1-yl}sulphonyl)methyl]pentanamide; and
(R/S)—N-{1-[4-(2,6-dimethyl-pyridin-4-ylmethoxy)-piperidine-1-sulphonylmethyl]-4-pyrimidin-2-yl-butyl)-N-(hydroxy)formamide.

In another aspect the present invention provides a process for the preparation of a compound of formula (1) or a pharmaceutically acceptable salt or in vivo hydrolysable ester thereof wherein Z is —N(OH)CHO, which process comprises the steps of:

converting a hydroxylamine of formula (2) into a compound of formula (1); 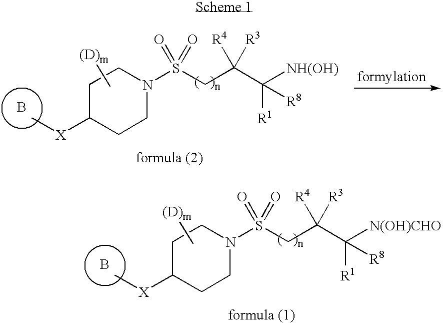
and thereafter if necessary:

i) converting a compound of formula (1) into another compound of formula (1);

ii) removing any protecting groups;

iii) forming a pharmaceutically acceptable salt or in vivo hydrolysable ester.

Formylation may be suitably performed by adding a preformed mixture of acetic acid (8 equivalents) and formic acid (excess) to formula (2) in tetrahydrofuran or dichloromethane and stirring the solution for l5hours at temperatures ranging from 0° C. to room temperature followed by stirring in methanol. Alternatively a formylation method described in J.Med.Chem., 2002, 45, 219 using trifluoroethylformate can be used.

This process may further comprise a process for the preparation of a hydroxylamine of formula (2):
when n is 0 and R⁴ is hydrogen (indicated as a compound of formula (2′)),which process comprises:

b) converting an alkene of formula (3) into a hydroxylamine of formula (2′);

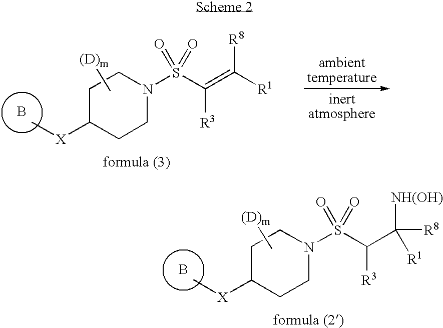

Suitable reagents for such a conversion include aqueous hydroxylamine in tetrahydrofuran under an argon atmosphere.

The alkene of formula (3) when R⁸ is hydrogen can be prepared by the reaction of a compound of formula (4′) with a compound of formula (5) under Wadsworth-Emmons or Peterson reaction conditions;

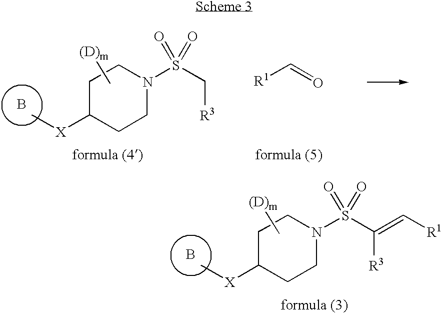

Wadsworth-Emmons or Peterson reactions involve the forming of the anion of formula (4′) with 2 equivalents of lithium bis(trimethylsilyl)amide or sodium hydride or lithium diisopropylamide in tetrahydrofuran at temperatures of −78° C. to 0° C. and reacting this with 1 equivalent of diethylchlorophosphate (Wadsworth Emmons) or 1 equivalent of trimethylsilyl chloride (Peterson). After 1 hour an aldehyde (1.1 equivalent) in tetrahydrofuran is added to the resultant anion described and reacted at room temperature over 15 hours.

The alkene of formula (3) can also be prepared by the reaction of a compound of formula (4′) with a compound of formula (6) as illustrated by scheme 4;

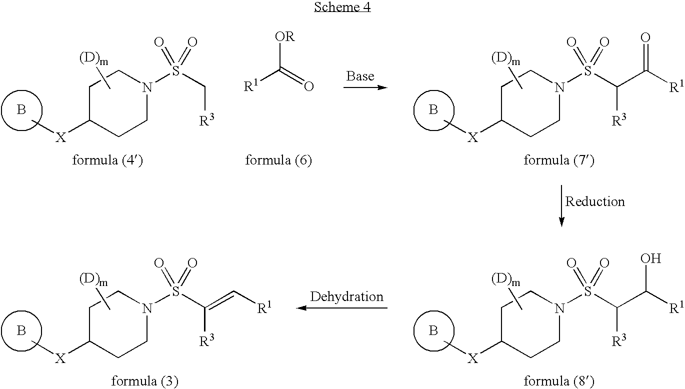

Suitable bases include lithium bis(trimethylsilyl)amide, sodium hydride or lithium diisopropylamide in tetrahydrofuran at temperatures of −78° C. to 0° C. to form the anion. Suitable reducing agents for the reduction step include sodium borohydride in ethanol or borane-dimethylsulphide complex or borane-tetrahydrofuran complex in tetrahydrofuran at room temperature. Suitable dehydration reagents for the dehydration step include methanesulphonyl chloride or tosyl chloride and triethylamine in dichloromethane at room temperature.

Or a process for the preparation of a hydroxylamine of formula (2):
when n is 0 (indicated as a compound of formula (2#)) may comprise;
c) i) reacting a compound of formula (4″) (see scheme 13 for its preparation) with R¹COOR, R¹COCl or activated R¹COOR to yield a ketone of formula (7″) (where R is C₁₋₂₀alkyl e.g. methyl, ethyl or arylC₁₋₄alkyl e.g. benzyl);
ii) reducing the ketone of formula (7″) to yield an alcohol of formula (8″);
iii) converting —OH group of the alcohol of formula (8″) into a leaving group (L) such as a halide, mesylate, tosylate etc. (see compound of formula (9″);
iv) displacing the leaving group with aqueous hydroxylamine to yield a hydroxylamine of formula (2#);

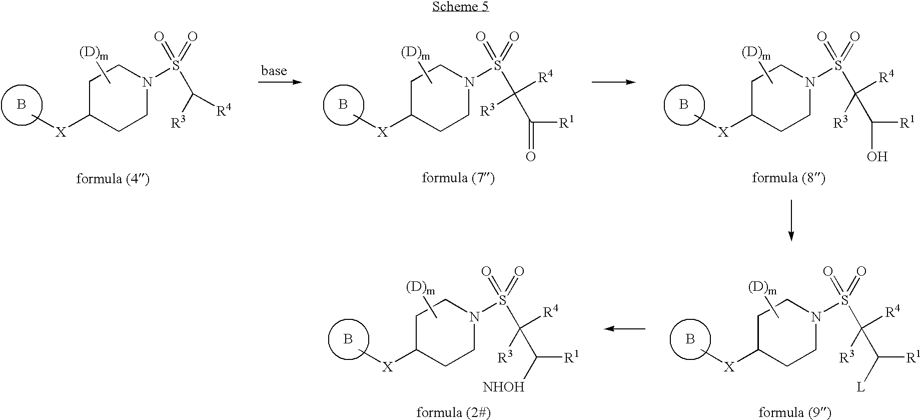

A ketone of formula (7″) may additionally be prepared by the process illustrated in scheme 6:

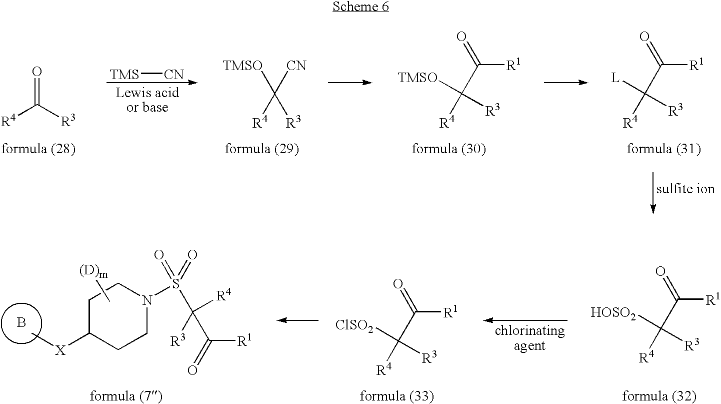

The silyl group present in the compound of formula (30) can be removed by tetrabutyl ammonium fluoride. Suitable leaving groups (L) are halo, mesyl and tosyl. A suitable chlorinating agent is POCl₃. A compound of formula (7″) is prepared in the last step by reacting the compound of formula (33) with the appropriate piperidine reagent.

Or a process for the preparation of a hydroxylamine of formula (2):
when n is 1 and R³ and R⁴ are both hydrogen (indicated as a compound of formula (2**)) may further comprise:
d) i) reacting a compound of formula (4″) with a compound of formula (10) (either an epoxide or equivalent) to yield an alcohol of formula (8**);
ii) converting —OH group of the alcohol of formula (8**) into a leaving group such as a halide, mesylate, tosylate etc. (see compound of formula (9**);
iii) displacing the leaving group with aqueous hydroxylamine to yield a hydroxylamine of formula (2**);

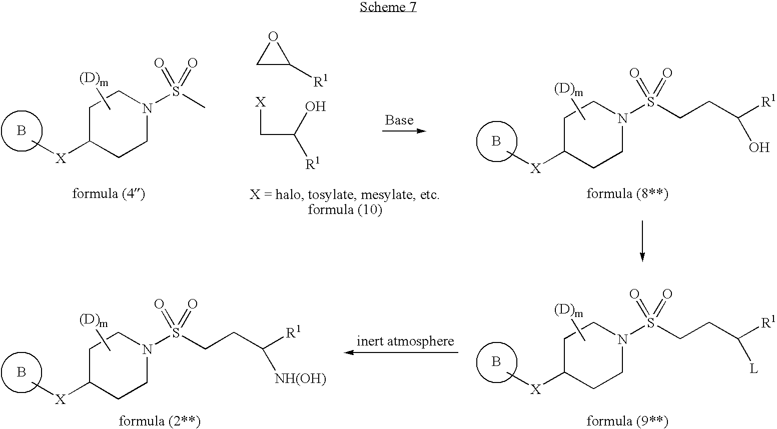

Suitable bases are lithium bis(trimethylsilyl)amide and lithium diisopropylamide at temperatures from −78° C. to 0° C. Suitable leaving groups (L) are chloro, bromo, iodo, methanesulphonyl and tosyl and these would be formed from the alcohol by treatment with methanesulphonyl chloride and pyridine in dichloromethane (mesylate), tosyl chloride and pyridine in dichloromethane (tosylate), triphenylphosphine and carbon tetrabromide (bromo); the chloro, bromo and iodo derivatives could also be prepared from the mesylate or tosylate by addition of a suitable halide source, e.g. tetrabutylammonium iodide or sodium iodide or lithium chloride in a solvent such as acetone.

Or a process for the preparation of a hydroxylamine of formula (2):
when n is 1, indicated as a compound of formula (2ˆ), may further comprise:
e) i) reacting a compound of formula (4″) with a compound of formula (11) to yield an ester of formula (12ˆ);
ii) converting the ester of formula (12ˆ) into an alcohol of formula (13ˆ);
iii) displacing the —OH group with aqueous hydroxylamine to yield a hydroxylamine of formula (2ˆ);

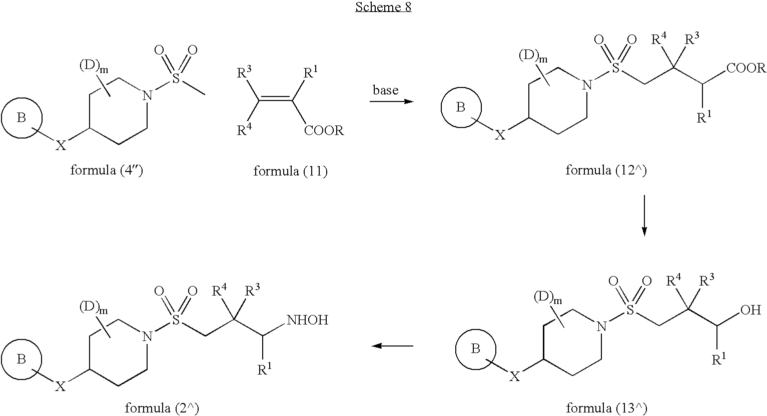

The group —COOR of formula (12ˆ) is representative of an ester wherein R may be C₁₋₂₀ alkyl, e.g. methyl, ethyl or arylC₁₋₄alkyl, e.g. benzyl. Baeyer-Villiger reaction conditions such as a peracid e.g. 3-chloroperoxybenzoic acid in dichloromethane are suitable for the is conversion of the ester group into the alcohol group. It may be appropriate to convert the alcohol group into a leaving group such as bromo, iodo, mesyl and tosyl, before displacement with aqueous hydroxylamine.

In another aspect the present invention provides a process for the preparation of a compound of formula (1) or a pharmaceutically acceptable salt or in vivo hydrolysable ester thereof wherein Z is —CONR¹⁵OH, which process comprises:

a) converting an acid of formula (14) into a compound of formula (1); 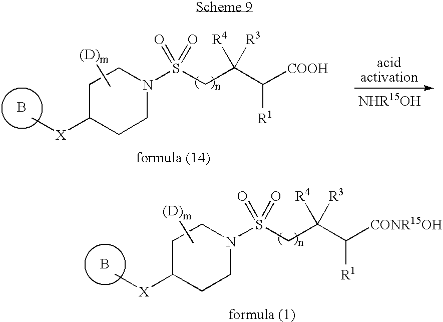
and thereafter if necessary:

i) converting a compound of formula (1) into another compound of formula (1);

ii) removing any protecting groups;

iii) forming a pharmaceutically acceptable salt or in vivo hydrolysable ester.

The acid of formula (14) may be suitably activated by conversion to an acid halide, such as the acid chloride or to an activated ester using carbonyldiimidazole, a carbodiimide or a pentafluorophenyl ester. Alternatively when the acid of formula (14) is an ester e.g. the methyl or ethyl ester, it can be converted directly to a compound of formula (1) by reaction with NHR¹⁵OH.

Also provided is a process for the preparation of an acid of formula (14) which process comprises;

b) reacting a compound of formula (4″) with an alkene of formula (11) to yield an ester of formula (12ˆ) which is hydrolysed to an acid of formula (14′) where an acid of formula (14′) is an acid of formula (14) wherein n is 1 and R⁸ is hydrogen;

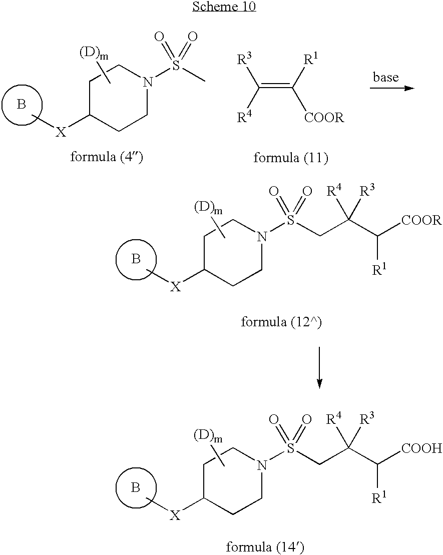

Suitable bases able to deprotonate a compound of formula (4″) are butyllithium, lithium diisopropylamide, lithium bis(trimethylsilyl)amide followed by the addition of a copper salt e.g. copper bromide-dimethylsulphide complex, copper iodide, in solvents such as dimethylsulphide, ether, tetrahydrofuran at temperatures from −78° C. to room temperature.

Or a process for the preparation of an acid of formula (14) comprises;

c) reacting a compound of formula (4″) with a compound of formula (15) to yield an acid of formula (14**) which is an acid of formula (14) wherein n is 0, R³ is hydrogen and R⁴ is hydrogen;

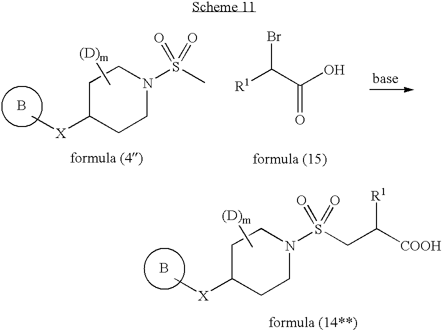

Suitable bases to deprotonate formula (4″) include lithium bis(trimethylsilyl)amide, lithium diisopropylamide and sodium hydride in solvents such as tetrahydrofuran and ether at temperatures from −78° C. to 0° C.

In another aspect the present invention provides a process for the preparation of a compound of formula (1) or a pharmaceutically acceptable salt or in vivo hydrolysable ester thereof wherein Z is —CONR¹⁵OH, R⁸ is hydrogen and n is 0, which process comprises steps as outlined in scheme 12:

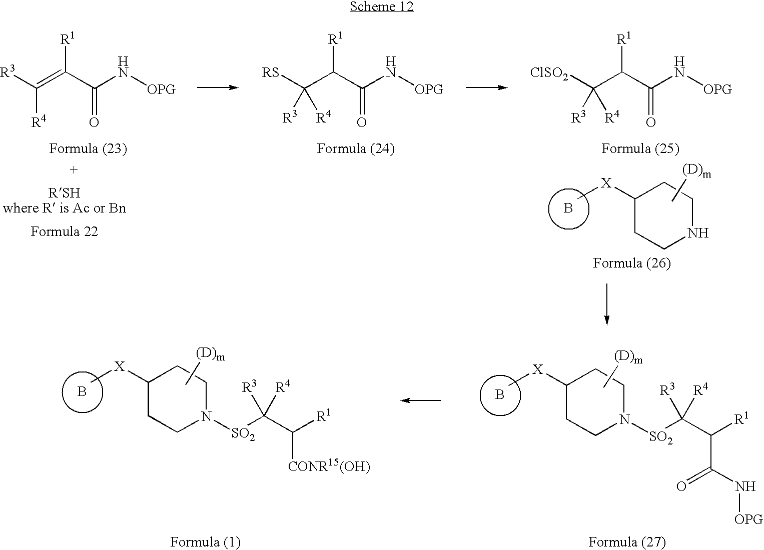

The process of scheme 12 comprises the steps of:
i) reacting a thiol of formula (22) with an acrylate of formula (23) at temperatures of 0° C. to 70° C. to yield a thioether of formula (24);
ii) oxidising the thioether of formula (24) to a sulphonyl chloride of formula (25) by bubbling chlorine gas onto a solution of the thioether in acetic acid at temperatures of 0° C. to room temperature;
iii) reacting the sulphonyl choride of formula (25) with a piperidine of formula (26) under standard sulphonamide conditions (e.g. triethylamine in dichloromethane at temperatures from 0° C. to 50° C.) to yield a compound of formula (27);
iv) removing the protecting group to yield a compound of formula (1).

The protecting group (PG) may be benzyl- or 2,4-dimethoxybenzyl. The former can be removed by treatment with hydrogen/ palladium and the latter by treatment with mild acid (see Tetrahedron Letters, 1998, 39(43), 7865).

The process of scheme 12 may further comprise if necessary:
v) converting a compound of formula (1) into another compound of formula (1);
vi) removing any other protecting groups;
vii) forming a pharmaceutically acceptable salt or in vivo hydrolysable ester.

In another aspect of the invention, there is provided a process for the preparation of a compounds of formula (4), formula (4′) and formula (4″) which process comprises;
i) reacting a compound of formula (16) where t is 1 or if t is 0 where B is an activated halo heterocyclyl with a compound of formula (17) (wherein Q is S or O), in the presence of a base to deprotonate the compound of formula (17), to yield a compound of formula (18);
ii) removing the protecting group (G) from the compound of formula (18) to yield a compound of formula (19);.
iii) reacting the compound of formula (19) with a suitable reagent to yield a compound of formula (4) wherein X is —(CR⁹R¹⁰)ₜ-Q-(CR¹¹R¹²)ᵤ; and
iv) oxidising Q where Q is S as required.

When R⁴ is hydrogen a compound of formula (4′) is produced and when R³ and R⁴ are both hydrogen compound of formula (4″) is produced;

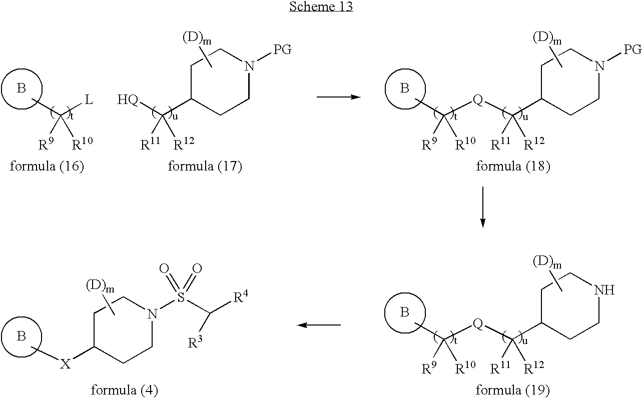

Compounds of formula (4), formula (4′) and formula (4″) may also be prepared by a process which comprises;
i) reacting a compound of formula (20) (wherein Q is S or O) with a compound of formula (21), in the presences of a base to yield a compound of formula (18);
ii) removing the protecting group (PG) from the compound of formula (18) to yield a compound of formula (19);.
iii) reacting the compound of formula (19) with a suitable reagent to yield a compound of formula (4) wherein X is —(CR⁹R¹⁰)ₜ-Q-(CR¹¹R¹²)ᵤ; and
iv) oxidising Q where Q is S as required.

When R⁴ is hydrogen a compound of formula (4′) is produced and when R³ and R⁴ are both hydrogen compound of formula (4″) is produced;

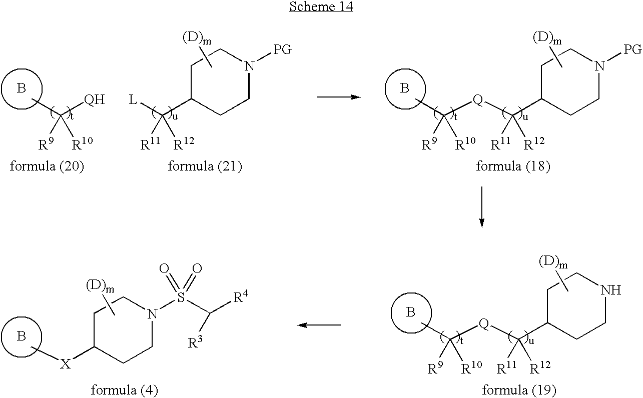

In both schemes 13 and 14: L is a suitable leaving group such as halo (chloro, bromo, iodo), hydroxy, mesyl or tosyl; suitable bases to deprotonate a compound of formula (17) and formula (20) include sodium hydride, lithium diisopropylamide, butyllithium and lithium bis(trimethylsilyl)amide; suitable reaction conditions for a) are temperatures ranging from −78° C. to 70° C. and an aprotic solvent, e.g. tetrahydrofuran under argon; suitable protecting groups (PG) include Boc (t-butoxycarbonyl), CBz (carbonyloxybenzyl) groups and mesyl or another alkylsulphonyl. In the case where PG is alkylsulphonyl, reaction of formula (16) and (17) and of formula (20) and formula (21) directly produces a compound of formula (4). A compound of formula (18) can be converted to formula (19) by treatment with acid (Boc) or hydrogen/ palladium (CBz). A compound of formula (19) can be converted to a compound of formula (4) by treatment with an alkylsulfonylchloride in the presence of a base such as pyridine in a solvent such as dichloromethane. In the case where t=0, Q is O, L is OH and B is aromatic, Mitsunobu conditions can be used to form a compound of formula (18), i.e. a compound of formula (16) or formula (20) would be reacted with a mixture of diethyl azodicarboxylate or diisopropylazodicarboxylate and triphenylphosphine and a compound of formula (17) or formula (21) to give a compound of formula (4). In addition PG could also be a protected hydroxamic acid or reverse hydroxamate. Thus reaction of formula (16) and (17) and of formula (20) and (21) would deliver a protected version of formula (1) which could then be deprotected.

A compound of formula (1) can be prepared by removal of a protecting group on the zinc binding group directly. The protecting group (PG) can be benzyl or 2,4-dimethoxybenzyl. The former can be removed by treatment with hydrogen/palladium and the latter by treatment with mild acid (see Tetrahedron Letters, 1998, 39(43), 7865). The required protected hydroxamic acid or reverse hydroxamate can be obtained by using a suitably protected hydroxylamine earlier in the synthesis.

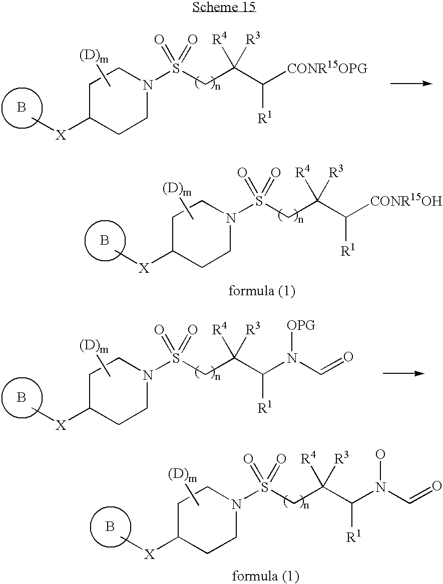

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Priedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid such as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses metalloproteinases inhibitory activity, and in particular TACE inhibitory activity. This property may be assessed, for example, using the procedure set out below.

Isolated Enzyme Assays

Matrix Metalloproteinase Family Including for Example MMP13.

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using 1 mM amino phenyl mercuric acid (APMA), 20 hours at 21° C.; the activated MMP13 (11.25 ng per assay) is incubated for 4-5 hours at 35° C. in assay buffer (0.1M Tris-HCl, pH 7.5 containing 0.1M NaCl, 20 mM CaCl₂, 0.02 mM ZnCl and 0.05% (w/v) Brij 35 using the synthetic substrate 7-methoxycoumarin-4-yl)acetyl.Pro.Leu.Gly.Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}−Fluorescence_{background}] divided by the [Fluorescence_{minus inhibitor}−Fluorescence_{background}].

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNFα convertase enzyme (TACE) may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate 4′,5′-Dimethoxy-fluoresceinyl Ser.Pro.Leu.Ala.Gln.Ala.Val.Arg.Ser.Ser.Ser.Arg.Cys(4-(3-succinimid-1-yl)-fluorescein)-NH₂ in assay buffer (50 mM Tris HCl, pH 7.4 containing 0.1% (w/v) Triton X-100 and 2 mM CaCl₂), at 26° C. for 4 hours. The amount of inhibition is determined as for MMP13 except λex 485 nm and λem 538 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(pmc or Pbf), Ser^{9,10,11}(Trityl), Cys¹³(Trityl). Following assembly, the N-terminal Fmoc-protecting group was removed by treating the Fmoc-peptidyl-resin with in DMF. The amino-peptidyl-resin so obtained was acylated by treatment for 1.5-2hours at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-BPLC and finally isolated by freeze-drying from aqueous acetic acid. The product was characterised by MALDI-TOF MS and amino acid analysis.

The compounds of the invention have been found to be active against TACE at 0.1 nM to 50 μM, and in particular 10 μM of compound 25 gave 97% inhibition and 10 μM of compound 42 gave 99% inhibition.

Natural Substrates

The activity of the compounds of the invention as inhibitors of aggrecan degradation may be assayed using methods for example based on the disclosures of E. C. Arner et al., (1998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/Tissue Based Activity Test as an Agent to Inhibit Membrane Sheddases such as TNF Convertase

The ability of the compounds of this invention to inhibit the cellular processing of TNFα production may be assessed in THP-1 cells using an ELISA to detect released TNF essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole Blood TNF Sheddase Activity

The ability of the compounds of this invention to inhibit TNFα production is assessed in a human whole blood assay where LPS is used to stimulate the release of TNFα. 160 μl of heparinized (10 Units/ml) human blood obtained from volunteers, was added to the plate and incubated with 20 μl of test compound (duplicates), in RPMI1640+bicarbonate, penicillin, streptomycin, glutamine and 1% DMSO, for 30 minutes at 37° C. in a humidified (5% CO₂/95% air) incubator, prior to addition of 20 μl LPS (E. coli. 0111:B4; final concentration 10 μg/ml). Each assay includes controls of neat blood incubated with medium alone or LPS (6 wells/plate of each). The plates are then incubated for 6 hours at 37° C. (humidified incubator), centrifuged (2000 rpm for 10 min; 4° C.), plasma harvested (50-100 μl) and stored in 96 well plates at −70° C. before subsequent analysis for TNFα concentration by ELISA.

Test as an Agent to Inhibit in Vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

In vivo Assessment

Test as an Anti-TNF Agent

The ability of the compounds of this invention as in vivo TNFα inhibitors is assessed in the rat. Briefly, groups of female Wistar Alderley Park (AP) rats (90-100 g) are dosed with compound (5 rats) or drug vehicle (5 rats) by the appropriate route e.g. peroral (p.o.), intraperitoneal (i.p.), subcutaneous (s.c.) 1 hour prior to lipopolysaccharide (LPS) challenge (30 μg/rat i.v.). Sixty minutes following LPS challenge rats are anaesthetised and a terminal blood sample taken via the posterior vena cavae. Blood is allowed to clot at room temperature for 2 hours and serum samples obtained. These are stored at −20° C. for TNFα ELISA and compound concentration analysis.

Data analysis by dedicated software calculates for each compound/dose:
Percent inhibition of TNFα=Mean TNFα (Vehicle control)−Mean TNFα (Treated)×100/Mean TNFα (Vehicle control)
Test as an Anti-Arthritic Agent

Activity of a compound as an anti-arthritic is tested in the collagen-induced arthritis (CIA) as defined by D. E. Trentham et al., (1977) J. Exp. Med. 146,:857. In this model acid soluble native type II collagen causes polyarthritis in rats when administered in Freunds incomplete adjuvant. Similar conditions can be used to induce arthritis in mice and primates.

Pharmaceutical Compositions

According to a further aspect of the invention there is provided a pharmaceutical composition which comprises a compound of formula (1), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository. The composition may also be a form suitable for inhalation.

In general the above compositions may be prepared in a conventional manner using conventional excipients.

The pharmaceutical compositions of this invention will normally be administered to humans so that, for example, a daily dose of 0.5 to 75 mg/kg body weight (and preferably 0.5 to 30 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention.

Therefore in a further aspect of the present invention there is provided a compound of formula (1), or a pharmaceutically acceptable salt or in vivo hydrolysable ester thereof, as defined hereinbefore, for use in a method of treatment of a warm-blooded animal such as man by therapy.

Also provided is a compound of formula (1), or a pharmaceutically acceptable salt or in vivo hydrolysable ester thereof, as defined hereinbefore, for use in a method of treating a disease condition mediated by one or more metalloproteinase enzymes and in particular a disease condition mediated by TNFα.

Further provided is a compound of formula (1), or a pharmaceutically acceptable salt or in vivo hydrolysable ester thereof, as defined hereinbefore, for use in a method of treating inflammatory diseases, autoimmune diseases, allergic/atopic diseases, transplant rejection, graft versus host disease, cardiovascular disease, reperfusion injury and malignancy in a warm-blooded animal such as man. In particular a compound of formula (1), or a pharmaceutically acceptable salt or in vivo hydrolysable ester thereof, as defined hereinbefore, is provided for use in a method of treating rheumatoid arthritis, Crohn's disease and psoriasis, and especially rheumatoid arthritis. A pharmaceutically acceptable salt or in vivo hydrolysable ester thereof, as defined hereinbefore, is also provided for use in a method of treating a respiratory disorder such as asthma or COPD.

According to an additional aspect of the invention there is provided a compound of formula (1), or a pharmaceutically acceptable salt or in vivo hydrolysable ester thereof, as defined hereinbefore, for use as a medicament.

Also provided is a compound of formula (1), or a pharmaceutically acceptable salt or in vivo hydrolysable ester thereof, as defined hereinbefore, for use as a medicament in the treatment of a disease condition mediated by one or more metalloproteinase enzymes and in particular a disease condition mediated by TNFα.

Further provided is a compound of formula (1), or a pharmaceutically acceptable salt or in vivo hydrolysable ester thereof, as defined hereinbefore, for use as a medicament in the treatment of inflammatory diseases, autoimmune diseases, allergic/atopic diseases, transplant rejection, graft versus host disease, cardiovascular disease, reperfusion injury and malignancy in a warm-blooded animal such as man. In particular a compound of formula (1), or a 30 pharmaceutically acceptable salt or in vivo hydrolysable ester thereof, as defined hereinbefore, is provided for use as a medicament in the treatment of rheumatoid arthritis, Crohn's disease and psoriasis, and especially rheumatoid arthritis. A compound of formula (1), or a pharmaceutically acceptable salt or in vivo hydrolysable ester thereof, as defined hereinbefore, is also provided for use as a medicament in the treatment of a respiratory disorder such as asthma or COPD.

According to another aspect of the invention there is provided the use of a compound of formula (1), or a pharmaceutically acceptable salt or in vivo hydrolysable ester thereof, as defined hereinbefore in the manufacture of a medicament for use in the treatment of a disease condition mediated by one or more metalloproteinase enzymes and in particular a disease condition mediated by TNFα in a warm-blooded animal such as man.

Also provided is the use of a compound of formula (1), or a pharmaceutically acceptable salt or in vivo hydrolysable ester thereof, as defined hereinbefore in the manufacture of a medicament for use in the treatment of inflammatory diseases, autoimmune diseases, allergic/atopic diseases, transplant rejection, graft versus host disease, cardiovascular disease, reperfusion injury and malignancy in a warm-blooded animal such as man. In particular the use of a compound of formula (1), or a pharmaceutically acceptable salt or in vivo hydrolysable ester thereof, as defined hereinbefore, is provided in the manufacture of a medicament in the treatment of rheumatoid arthritis, Crohn's disease and psoriasis, and especially rheumatoid arthritis. The use of a compound of formula (1), or a pharmaceutically acceptable salt or in vivo hydrolysable ester thereof, as defined hereinbefore, is also provided in the manufacture of a medicament in the treatment of a respiratory disorder such as asthma or COPD.

According to a further feature of this aspect of the invention there is provided a method of producing a metalloproteinase inhibitory effect in a warm-blooded animal, such as man, in need of such treatment which comprises administering to said animal an effective amount of a compound of formula (1).

According to a further feature of this aspect of the invention there is provided a method of producing a TACE inhibitory effect in a warm-blooded animal, such as man, in need of such treatment which comprises administering to said animal an effective amount of a compound of formula (1).

According to this further feature of this aspect of the invention there is provided a method of treating autoimmune disease, allergic/atopic diseases, transplant rejection, graft versus host disease, cardiovascular disease, reperfusion injury and malignancy in a warm-blooded animal, such as man, in need of such treatment which comprises administering to said animal an effective amount of a compound of formula (1).

Also provided is a method of treating rheumatoid arthritis, Crohn's disease and psoriasis, and especially rheumatoid arthritis in a warm-blooded animal, such as man, in need of such treatment which comprises administering to said animal an effective amount of a compound of formula (1). Further provided is a method of treating a respiratory disorder such as asthma or COPD in a warm-blooded animal, such as man, in need of such treatment which comprises administering to said animal an effective amount of a compound of formula (1).

In addition to their use in therapeutic medicine, the compounds of formula (1) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;

(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glamorgan, UK. Where Flashmaster II is referred to, this means a UV driven automated chromatography unit supplied by Jones;

(iv) in general, the course of reactions was followed by TLC and reaction times are given for illustration only;

(v) yields, when given, are for illustration only and are not necessarily those which can be obtained by diligent process development; preparations were repeated if more material was required;

(vi) when given, ¹H NMR data is quoted and is in the form of delta values for major diagnostic protons, given in parts per million (ppm) relative to tetramethylsilane (TMS) as an internal standard, determined at 300 ME using perdeuterio DMSO (CD₃SOCD₃) as the solvent unless otherwise stated; coupling constants (J) are given in Hz;

(vii) chemical symbols have their usual meanings; SI units and symbols are used;

(viii) solvent ratios are given in percentage by volume;

(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;

(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and

(xi) the following abbreviations are used:
DMSO dimethyl sulphoxide;
DMF N-dimethylformamide;
DCM dichloromethane;
NMP N-methylpyrrolidinone;
DIAD Di-isopropylazodicarboxylate
LHMDS or LiHMDS Lithium bis(trimethylsilyl)amide
MeOH Methanol
RT Room temperature
TFA Trifluoroacetic acid
EtOH ethanol
EtOAc ethyl acetate.
THF tetrahydrofuran
TBDMS tertiarybutyldimethylsilyl
DIPEA diisopropylethylamine
MTBE methyltertiarybutylether

### Example 1

### (R/S)-1-[({4-[(2-Methylquinolin-4-yl)methloxy]piperidin-1-yl}sulphonyl)methyl]-4-pyrimidin-2-ylbutyl(hydroxy)formamide

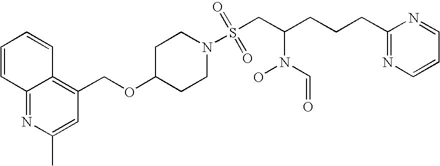

To a stirred solution of (R/S)-{1-[({4-[(2-methylquinolin-4-yl)methyloxy]piperidin-1-yl}sulphonyl)methyl]-4-pyrimidin-2-ylbutyl}hydroxylamine (150 mg, 0.27 mmol) in THF (4 ml), was added a preformed mixture of acetic anhydride (2001, 2.1 mmol) and formic acid (0.8 ml). The mixture was stirred at RT overnight. The solvents were removed by rotary evaporation, and the residue partitioned between EtOAc (50 ml) and sodium hydrogen carbonate (20 ml). The organic layer was dried (Na₂SO₄), concentrated and purified by chromatography (10 g silica bond elute, eluent 0→12% EtOH/DCM) to give (R/S)-1-[({4-[(2-methylquinolinyl-4-yl)methyloxy]piperidin-1-yl}sulphonyl)methyl]-4-pyrimidin-2-ylbutyl(hydroxy)formamide as a white foam (76mg, 0.14 mmol).

NMR: 1.7 (m, 6H), 1.9 (m, 2H), 2.6 (s, 3H), 2.85 (m, 2H), 3.1 (m, 3H), 3.4 (m, 4H), 3.7 (m, 1H), 5.0 (s, 2H), 7.3 (m, 1H), 7.45 (s, 1H), 7.5 (t, 1H), 7.7 (t, 1H), 8.1 (m, 3H), 8.7 (m, 2H), 9.7 (m, 1H); MS: 528.

The starting material (R/S)-{1-[({4-[(2-methylquinolinyl-4-yl)methyloxy]piperidin-1-yl}sulphonyl)methyl]-4-pyrimidin-2-ylbutyl}hydroxylamine was prepared as follows:

i) To a stirred suspension of 2-methylquinolin-4-ylcarboxylic acid (4 g, 21.4 mmol) in THF (100 ml) at RT was added lithium aluminium hydride (21.4 ml, 1.0M solution in THF, 21.4 mmol) dropwise over 20 minutes. After 16 h water (4 ml) was added cautiously followed by 2N NaOH (4 ml) and water (12 ml). The resulting gelatinous precipitate was filtered off and washed with THF. DCM (200 ml) was added to the filtrate and partitioned with saturated NaHCO₃ (2×75 ml). The organic layer was dried (MgSO₄), concentrated, triturated with DCM and filtered to give 2-methylquinolin-4-ylmethanol as a white powder (858 mg, 5 mmol). The mother liquours were purified by chromatography (20 g silica bond elute, eluent 0→5% EtOH/DCM) to give a further 610 mg of product (3.5 mmol). NMR: 2.6 (s, 3H), 5.0 (d, 2H), 5.5 (t, 1H), 7.4 (s, 1H), 7.5 (t, 1H), 7.7 (t, 1H) and 7.9 (m, 2H); MS: 174.

ii) To a suspension of 2-methylquinolin-4-ylmethanol (10 mg, 0.58 mmol) in DCM (5 ml) at RT was added triethylamine (0.24 ml, 1.74 mmol). The reaction mixture was then cooled to 0° C. and methanesulphonylchloride (0.05 ml, 0.64 mmol) was added dropwise. After 10 minutes the reaction mixture was concentrated and EtOAc (20 ml) was added and the organic layer partitioned with brine (10 ml), dried (MgSO₄), concentrated and purified by chromatography (10 g silica bond elute, eluent 5% MeOH/DCM) to give 2-methylquinolin-4-ylmethyloxysulphonylmethane (110 mg, 0.44 mmol). NMR: 2.7 (s, 3H), 3.35 (s, 3H), 5.75 (s, 2H), 7.5 (s, 1H), 7.6 (t, 1H), 7.75 (t, 1H), 8.0 (m, 2H): MS: 252.

iii) To a solution of tert-butyl 4-hydroxypiperidin-1-ylcarboxylate (1.75 g, 8.73 mmol) in DMF (20 ml) at 0° C. was added sodium hydride (419 mg, 60% dispersion in oil, 10.5 mmol). After 10 minutes a solution of 2-methylquinolin-4-ylmethyloxysulphonylmethane (2.19 g, 8.73 mmol) in DMP (10 ml) was added dropwise over 5 minutes at 0° C. After 5 hours the mixture was concentrated and the residue taken up in EtOAc (150 ml). The organic layer was washed with brine (50 ml), dried (Na₂S_{2l O}₄), concentrated and purified by chromatography (MPLC, eluting with 75% EtOAc/hexane) to give tert-butyl 4-(2-methylquinolin-4-ylmethyloxy)piperidin-1-ylcarboxylate (1.46 g, 4.1 mmol). MS: 357.

iv) To a solution of tert-butyl 4-(2-methylquinolin-4-ylmethyloxy)piperidin-1-ylcarboxylate (1.45 g, 4.1 mmol) in DCM (10 ml) at RT was added TFA (3 ml). After 15 hours the mixture was concentrated and azeotroped with toluene (×2) to give 4-(2-methylquinolin-4-ylmethyloxy)piperidine.di TFA salt (1.97 g, 4.1 mmol). MS: 257.

v) To a solution of 4-(2-methylquinolin-4-ylmethyloxy)piperidine.di TFA salt (2.49 g, 5.2 mmol) in DCM (40 ml) at 0° C. was added triethylamine (4.3 ml, 31 mmol) followed by dropwise addition of methanesulphonyl chloride (0.8 ml, 10.3 mmol) dropwise over 1 minuteand the reaction mixture was allowed to warm to RT. After 15 hours the mixture was diluted with DCM (60 ml), washed with water (30 ml), brine (25 ml), concentrated and purified by chromatography (MPLC, eluting with 100% EtOAc) to give 4-(2-methylquinolin-4-ylmethyloxy)-piperidin-1-ylsulphonylmethane (600 mg, 1.8 mmol) as a pale yellow solid. NMR: 1.6 (m, 2H), 2.0 (m, 2H), 2.65 (s, 3H), 2.85 (s, 3H), 3.0 (m, 2H), 3.3 (m, 2H), 3.7 (m, 1H), 5.0 (s, 2H), 7.4 (s, 1H), 7.5 (t, 1H), 7.7 (t, 1H), 7.9 (d, 1H) and 8.0 (d, 1H); MS: 335.

vi) To a stirred solution of 4-(2-methylquinolin-4-ylmethyloxy)-piperidin-1-ylsulphonylmethane (150 mg, 0.45 mmol) in THF (6 ml) at −10° C. under argon was added LHMDS (0.945 ml, 1M in THF, 0.945 mmol) and after a further 15 minutes, diethyl chlorophosphate (0.065 ml, 0.45 mmol) was added. After 1 hour a solution of 4-(2-pyrimidinyl)-butanal§ (75 mg, 0.495 mmol) in THF (0.2 ml) was added and the mixture allowed to warm to RT over the weekend. Saturated ammonium chloride (8 ml) was then added and the organic layer separated. The aqueous layer was re-extracted with EtOAc (8 ml) and the combined organics were concentrated and purified by chromatography (10 g silica bond elute, eluent 0→6% EtOH/DCM) to give E-1-{4-(2-methylquinolin-4-ylmethyloxy)-piperidin-1-ylsulphonyl}-5-(pyrimidin-2-yl)pent-1-ene as a pale yellow oil (0.13 g, 0.28 mmol). NMR: 1.2 (m, 1H), 1.7 (m, 2H), 1.9 (m, 4H), 2.3 (m, 1H), 2.65 (s, 3H), 2.9 (m, 4H), 3.3 (m, 1H), 3.65 (m, 1H), 4.0 (m, 1H), 5.0 (s, 2H), 6.5 (m, 2H), 7.3 (m, 1H), 7.4 (d, 1H), 7.5 (m, 1H), 7.7 (m, 1H), 7.9 (m, 1H), 8.0 (m, 1H) and 8.7 (m, 2H); MS: 467.

vii) To a stirred solution of the E-1-{4-(2-methylquinolin-4-ylmethyloxy)-piperidin-1-ylsulphonyl}-5-(pyrimidin-2-yl)pent-1-ene (125 mg, 0.27 mmol) in THF (7 ml) under argon was added hydroxylamine (50% solution in water, 3 ml) and the mixture stirred overnight. The mixture was poured into water (10 ml) and EtOAc (50 ml) and the partitioned organic layer was dried (MgSO₄) and concentrated to give (R/S)-{1-[({4-[(2-methylquinolin-4-yl)methyloxy]piperidin-1-yl}sulphonyl)methyl]-4-pyrimidin-2-ylbutyl}hydroxylamine (150 mg, 0.27 mmol); MS: 500.

§ 4-(2-pyrimidinyl)-butanal has been reported in the literature and has CAS registry number 260441-10-9 (CA Index Name: 2-pyrimidinebutanal).

### Example 2

### (R/S)-1-methyl-2-({4-[(2-methylquinolin-4-yl)methoxy]piperidin-1-yl}sulphonyl)ethyl(hydroxy)formamide

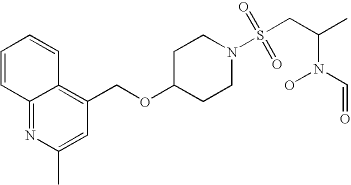

The procedure described in Example 1 was followed except that 4-(2-methylquinolin-4-ylmethyloxy)piperidin-1-ylsulphonylmethane (150 mg, 0.45 mmol) (synthesis described above) was reacted with acetaldehyde (0.028 ml, 0.495 mml) instead of 4-(2-pyrimidinyl)-butanal to give (R/S)-1-methyl-2-({4-[(2-methylquinolin-4-yl)methoxy]piperidin-1-yl}sulphonyl)ethyl(hydroxy)formamide (47 mg, 0.11 mmol). NMR: 1.2 (m, 3H), 1.7 (m, 2H), 2.0 (m, 2H), 2.65 (s, 3H), 3.05 (m, 3H), 3.4 (m, 4H), 3.7 (m, 1H), 5.0 (s, 2H), 7.4 (s, 1H), 7.5 (t, 1H), 7.7 (t, 1H) and 8.0 (m, 3H); MS: 422.

### Example 3

### (R/S)-1-pyrid-3-yl-2-({4-[(2-methylquinolin-4-yl)methoxy]piperidin-1-yl}sulphonyl)ethyl(hydroxy)formamide

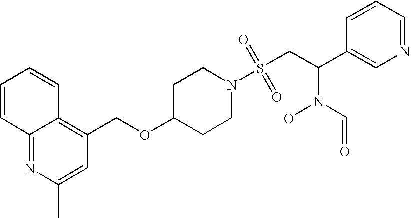

The procedure described in Example 1 was followed except that 4-(2-methylquinolin-4-ylmethyloxy)piperidin-1-ylsulphonylmethane (150 mg, 0.45 mmol) (synthesis described above) was reacted with pyrid-3-ylcarboxaldehyde (0.047 ml, 0.495 mml) instead of 4-(2-pyrimidinyl)-butanal to give (R/S)-1-pyrid-3-yl-2-({4-[(2-methylquinolin-4-yl)methoxy]piperidin-1-yl}sulphonyl)ethyl(hydroxy)formamide (74 mg, 0.15 mmol). NMR: 1.6 (m, 2H), 1.9 (m, 2H), 2.6 (m, 3H), 3.0 (m, 2H), 3.0 (m, 4H), 3.75 (m, 2H), 5.0 (s, 2H), 7.4 (m, 2H), 7.5 (t, 1H), 7.7 (t, 1H), 7.9 (m, 2H), 8.0 (d, 1H), 8.2 (s, 1H), 8.5 (m, 1H) and 8.6 (m, 1H), MS: 485.

### Example 4

### (R/S)-1-(1H-imidazol-4-yl)-2-({4-[(2-methylquinolin-4-yl)methoxy]piperidin-1-yl}sulphonyl)ethyl(hydroxy)formamide

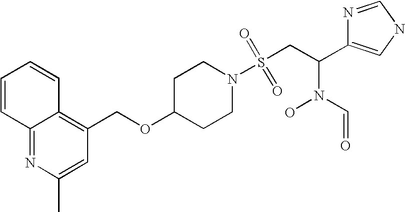

The procedure described in Example 1 was followed except that 4-(2-methylquinolin-4-ylmethyloxy)piperidin-1-ylsulphonylmethane (150 mg, 0.45 mmol) (synthesis described above) was reacted with imidazol-5-ylcarboxaldehyde (48 mg, 0.495 mml) instead of 4-(2-pyrimidinyl)-butanal to give (R/S)-1-(1H-imidazol-4-yl)-2-({4-[(2-methylquinolin-4-yl)methoxy]piperidin-1-yl}sulphonyl)ethyl(hydroxy)formamide, which was purified using a 10 g SCX column (eluting with 100% MeOH followed by 10% aqueous ammonia, MeOH) (39 mg, 0.15 mmol). NMR: 1.6 (m, 2H), 2.0 (m, 2H), 2.65 (s, 3H), 3.0 (m, 2H), 3.2 (m, 2H), 3.3 (m, 2H), 3.7 (m, 2H), 5.0 (s, 2H), 7.1 (s, 1H), 7.5 (m, 3H), 7.7 (m, 2H), and 8.0 (m, 3H); MS: 474.

### Example 5

### (R/S)-2-({4-[(2,5-dimethylbenzyl)oxy]piperidin-1-yl}sulphonyl)-1-pyrid-3-ylethyl(hydroxy)formamide

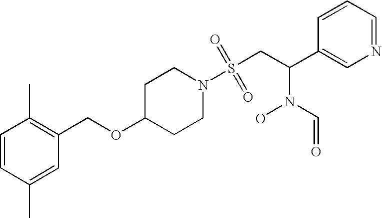

To a stirred solution of (R/S)-1-[({4-[(2,5-dimethylbenzyl)oxy]piperidin-1-yl}sulphonyl)methyl]pyrid-3-ylmethylhydroxylamine (130 mg, 0.31 mmol) in THF (2.5 ml), was added a preformed mixture of acetic anhydride (100 μl) and formic acid (0.4 ml). The mixture was stirred at RT overnight. The solvents were removed by rotary evaporation, and saturated sodium hydrogen carbonate (4 ml) was added. EtOAc (10 ml) was added and the mixture separated. The aqueous layer was re-extracted with EtOAc (5 ml) and the combined organic layer was concentrated in the presence of MeOH (10 ml) and purified by chromatography (10 g silica bond elute, eluent 0→100% EtOH/DCM followed by 5% EtOH/EtOAc) to give (R/S)-2-({4-[(2,5-dimethylbenzyl)oxy]piperidin-1-yl}sulphonyl)-1-pyrid-3-ylethyl(hydroxy)formamide as a foam (64 mg, 0.14 mmol). MS: 447.

The starting material (R/S)-1-[({4-[(2,5-dimethylbenzyl)oxy]piperidin-1-yl}sulphonyl)methyl]pyrid-3-ylmethylhydroxylamine was prepared as follows:

i) To a solution of tert-butyl 4-hydroxypiperidin-1-ylcarboxylate (4 g, 19.9 mmol) in DMF (100 ml) at RT was added sodium hydride (796 mg, 60% dispersion in oil, 19.9 mmol). After 1 hour 2,5-dimethylbenzyl chloride (2.94 ml, 19.9 mmol) was added dropwise. After 16 hours water was added (5 ml) and the DMF removed in vacuo. The mixture was partitioned between water (100 ml) and DCM (3×200 ml) and the combined organic layer was dried (MgSO₄), concentrated and purified by chromatography (MPLC, eluting with 0→20% EtOAc/DCM) to give tert-butyl 4-(2,5-dimethylbenzyloxy)piperidin-1-ylcarboxylate as a green oil (4.15 g, 13 mmol). NMR: 1.4 (m, 11H), 1.8 (m, 2H), 2.2 (d, 6H), 3.0 (m, 2H), 3.6 (m, 3H), 4.4 (s, 2H), 7.0 (m, 2H), 7.1 (s, 1H); MS: 320.

ii) To a solution of tert-butyl 4-(2,5-dimethylbenzyloxy)piperidin-1-ylcarboxylate (4.1 g, 12.85 mmol) in DCM (30 ml) was added TFA (3 ml) and the mixture stirred overnight at RT. TFA (3 ml) was added and the mixture stirred at 40° C. After 1 hour the mixture was concentrated and the residue azeotroped with toluene to give 4-(2,5-dimethylbenzyloxy)piperidine.TFA salt as a colourless oil (5.52 g, 12.85 mmol plus a small amount of toluene). NMR: 1.7 (m, 2H), 2.0 (m, 2H), 2.2 (s, 3H), 2.25 (s, 3H), 3.0 (m, 2H), 3.2 (m, 2H), 3.65 (m, 1H), 4.45 (s, 2H), 7.0 (m, 2H) and 7.1 (s, 1H): MS: 220.

iii) To a solution of 4-(2,5-dimethylbenzyloxy)piperidine.TFA salt (5.51 g, 12.85 mmol plus a small amount of toluene) in DCM (90 ml) at 0° C. was added triethylamine (8.59 ml, 61.6 mmol) followed by dropwise addition of methanesulphonyl chloride (1.05 ml, 13.6 mmol) over 5 minutes and the reaction mixture was allowed to warm to RT. After 63 hours the mixture was diluted with DCM (90 ml), washed with water (50 ml), brine (50 ml), dried (MgSO₄) and concentrated to give a light brown oil. The oil was triturated with EtOH (20 ml), filtered and washed with cold EtOH and concentrated to give 4-(2,5-dimethylbenzyloxy)piperidin-1-lsulphonylmethane as a white solid (2.63 g, 8.0 mmol). NMR: 1.6 (m, 2H), 1.9 (m, 2H), 2.2 (s, 3H), 2.25 (s, 3H), 2.85 (s, 3H), 3.0 (m, 2H), 3.55 (m, 1H), 4.45 (s, 2H), 7.0 (m, 2H) and 7.1 (s, 1H); MS: 298.

iv) To a stirred solution of 4-(2,5-dimethylbenzyloxy)piperidin-1-ylsulphonylmethane (200 mg, 0.67 mmol) in THF (8 ml) at −10° C. under argon was added LHMDS (1.48 ml, 1M in THF, 1.48 mmol) and after a further 15 minutes, diethyl chlorophosphate (0.086 ml, 0.67 mmol) was added. After 1 hour a solution of pyrid-3-ylcarboxaldehyde (70 mg, 0.74 mmol) in THF (0.5 ml) was added and the mixture allowed to warm to RT over the weekend. Saturated ammonium chloride (10 ml) was then added and the organic layer separated. The aqueous layer was re-extracted with EtOAc (10 ml) and the combined organics were concentrated and purified by chromatography (10 g silica bond elute, eluent 5→100% EtOAc/hexane) to give E-1-{4-(2,5-dimethylbenzyloxy)-piperidin-1-ylsulphonyl}-2-pyrid-3-ylethene as a clear gum (0.09 g, 0.23 mmol). MS: 387.

v) To a stirred solution of the E-1-{4-(2,5-dimethylbenzyloxy)-piperidin-1-ylsulphonyl}-2-pyrid-3-ylethene (90 mg, 0.23 mmol) in THF (5 ml) under argon was added hydroxylamine (50% solution in water, 2 ml) and the mixture stirred for 4 days. The mixture was poured into water (2 ml) and EtOAc (10 ml) and the partitioned organic layer was dried (MgSO₄) and concentrated to give (R/S)-1-[({4-[(2,5-dimethylbenzyl)oxy]piperidin-1-yl}sulphonyl)methyl]pyrid-3-ylmethyl hydroxylamine (96 mg, 0.23 mmol); MS: 420.

### Examples 6-9

The procedures described in Example 5 were followed using 4-(2,5-dimethylbenzyloxy)piperidin-1-ylsulphonylmethane (decribed above) with the aldehyde highlighted in the table in place of pyrid-3-ylcarboxaldehyde.

| | | | MS of |
|---|---|---|---|
| | | Aldehyde starting | final |
| Example | Structure and Name | material | product |
| 6 | 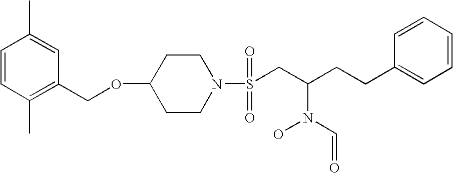 | 3-Phenylpropan-1-al | 475 |
| | (R/S)-[1-({[4-(2,5- | | |
| | Dimethylbenzyloxy)piperidin-1- | | |
| | yl]sulphonyl}methyl)-3- | | |
| | phenylpropyl]hydroxyformamide | | |
| 7 | 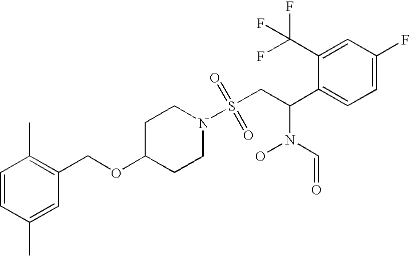 | 4-Fluoro-2- trifluoromethylbenzal dehyde 533 | |
| | (R/S)-2-({4-[(2,5- | | |
| | dimethylbenzyl)oxy]piperidin-1- | | |
| | yl}sulphonyl)-1-[4-fluoro-2- | | |
| | (trifluoromethyl)phenyl]ethyl(hy | | |
| | droxy)formamide | | |
| 8 | 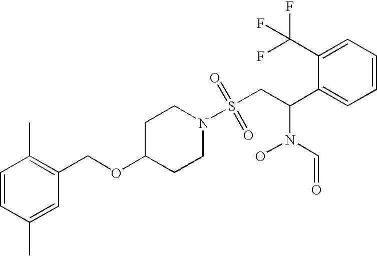 | 2-Trifluoromethylbenzal dehyde | 515 |
| | (R/S)-2-({4-[(2,5- | | |
| | dimethylbenzyl)oxy]piperidin-1- | | |
| | yl}sulphonyl)-1-[2- | | |
| | (trifluoromethyl)phenyl]ethyl(hy | | |
| | droxy)formamide | | |
| 9 | 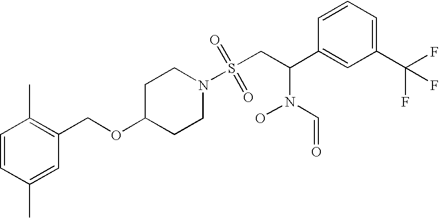 | 3-Trifluoromethylbenzal dehyde | 515 |
| | (R/S)-2-({4-[(2,5- | | |
| | dimethylbenzyl)oxy]piperidin-1- | | |
| | yl}sulphonyl)-1-[3- | | |
| | (trifluoromethyl)phenyl]ethyl(hy | | |
| | droxy)formainide | | |

### Example 10

### (R/S)-2-({4-[(2,5-Dimethylphenoxy)methyl]piperidin-1-yl}sulphonyl)-1-(4-fluorophenyl)ethyl(hydroxy)formamide

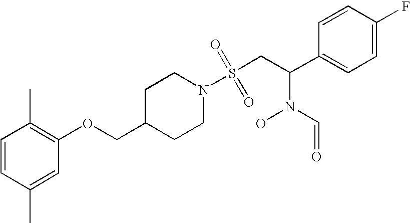

To a stirred solution of (R/S)-2-({4-[(2,5-dimethylphenoxy)methyl]piperidin-1-yl}sulphonyl)-1-(4-fluorophenyl)ethylhydroxylamine (185 mg, 0.42 mmol) in THF (5 ml), was added a preformed mixture of acetic anhydride (200 μl, 2.1 mmol) and formic acid (0.8 ml). The mixture was stirred at RT overnight. The solvents were removed by rotary evaporation, and the residue was stirred in a mixture of EtOAc and saturated sodium hydrogen carbonate for 2 h, then the layers were separated and the organic layer was washed with water, dried (Na₂SO₄), concentrated and purified by chromatography (10 g silica bond elute, eluent 50→100% EtOAc/Hexane; then on MPLC, eluent 25→75% EtOAc/DCM; then on a 20 g silica bond elute, eluent 30→100% EtOAc/DCM) to give (R/S)-2-({4-[(2,5-dimethylphenoxy)methylpiperidin-1-yl}sulphonyl)-1-(4-fluorophenyl)ethyl(hydroxy)formamide as a pale-yellow foam (90 mg, 0.19 mmol). NMR: 1.3 (m, 4H), 1.8 (m, 3H), 2.1 (s, 3H), 2.25 (s, 3H), 2.8 (m, 2H), 3.3 (m, 1H), 3.6 (m, 2H), 3.8 (d, 2H), 6.6 (d, 1H), 6.7 (s, 1H), 7.0 (d, 1H), 7.2 (t, 2H), 7.5 (m, 2H), 8.2 (s, 1H); MS: 465.

The starting material (R/S)-2-({4-[(2,5-dimethylphenoxy)methyl]piperidin-1-yl}sulphonyl)-1-(4-fluorophenyl)ethylhydroxylamine was prepared as follows:

i) To a stirred solution of piperidin-4-ylmethanol (2.85 g, 24.8 mmol) dissolved in DMF (130 ml) at 0° C. was added triethylamine (13.8 ml, 99.2 mmol) followed by methanesulphonyl chloride (4.8 ml, 62 mmol) dropwise over 5 minutes and the reaction mixture allowed to warm to RT. After 16 hours the reaction mixture was concentrated and the residue was partitioned between water (50 ml) and DCM (3×150 ml). The combined organics were dried (Na₂SO₄), concentrated and triturated with EtOH to give 4-(methanesulphonyloxymethyl)piperidin-1-ylsulphonylmethane as a pale yellow solid (3.2 g, 11.8 mmol). NMR: 1.3 (m, 2H), 1.8 (m, 3H), 2.7 (m, 2H), 2.85 (s, 3H), 3.15 (s, 3H), 3.6 (m, 2H), 4.1 (d, 2H); MS: 272.

ii) To a stirred solution of 2,5-dimethylphenol (298 mg, 2.4 mmol) and 4-(methanesulphonyloxymethyl)piperidin-1-ylsulphonylmethane (600 mg, 2.2 mmol) in DMF (10 ml) was added sodium hydride (132 mg, 3.6 mmol) and the mixture was heated to 60° C. After 1 hour the mixture was cooled, concentrated and triturated with EtOH to give a brown solid. This solid was treated with saturated sodium hydrogen carbonate, filtered and dried under vacuum to give 4-(2,5-dimethylphenyloxymethyl)piperidin-1-ylsulphonylmethane as a white powdery solid (518 mg, 1.95 mmol). NMR: 1.4 (m, 2H), 1.9 (m, 3H), 2.1 (s, 3H), 2.25 (s, 3H), 2.7 (m, 2H), 2.85 (s, 3H), 3.6 (m, 2H), 3.8 (d, 2H), 6.6 (d, 1H), 6.7 (s, 1H) and 7.0 (1H); MS: 298.

iii) To a stirred solution of 4-(2,5-dimethylphenyloxymethyl)piperidin-1-ylsulphonylmethane (200 mg, 0.67 mmol) in THF (8 ml) at −10° C. under argon was added LHMDS (1.48 ml, 1M in THF, 1.48 mmol) and after a further 15 minutes, diethyl chlorophosphate (0.097 ml, 0.67 mmol) was added. After 1.5 hours 4-fluorobenzaldehyde (0.079 ml, 0.74 mmol) was added and the mixture allowed to warm to RT overnight. Saturated ammonium chloride (10 ml) was then added and the organic layer separated. The aqueous layer was re-extracted with EtOAc (10 ml) and the combined organics were dried (Na₂SO₄), concentrated and purified by chromatography (10 g silica bond elute, eluent 5→10% EtOAc/Hexane) to give E-1-{4-(2,5-dimethylphenyloxymethyl)-piperidin-1-ylsulphonyl}-2-(4-fluorophenyl)ethene as a white waxy solid (0.21 g, 0.5 mmol). NMR: 1.4 (m, 2H), 1.9 (m, 3H), 2.0 (s, 3H), 2.25 (s, 3H), 2.7 (m, 2H), 3.6 (d, 2H), 3.8 (d, 2H), 6.6 (d, 1H), 6.7 (s, 1H), 6.95 (d, 1H), 7.2 (m, 3H), 7.4 (m, 1H) and 7.8 (m, 2H); MS: 404.

iv) To a stirred solution of the E-1-{4-(2,5-dimethylphenyloxymethyl)-piperidin-1-ylsulphonyl}-2-(4-fluorophenyl)ethene (200 mg, 0.5 mmol) in THF (5 ml) under argon was added hydroxylamine (50% solution in water, 1.5 ml) and the mixture stirred over the weekend. Saturated ammonium chloride (8 ml) was added and the layers separated. The aqueous layer was partitioned with DCM (10 ml) and the combined organics were dried (Na₂SO₄), concentrated and purified by chromatography (10 g silica bond elute, eluent 50→100% EtOAc/Hexane; then 20 g silica bond elute, eluent 50→100% EtOAc/Hexane) to give (R/S)-2-({4-[(2,5-dimethylphenyloxy)methyl]piperidin-1-yl}sulphonyl)-1-(4-fluorophenyl)ethylhydroxylamine as a colourless oil (170 mg, 0.39 mmol); NMR: 1.3 (m, 2H), 1.8 (m, 3H), 2.1 (s, 3H), 2.25 (s, 3H), 2.8 (m, 2H), 3.6 (m, 3H), 3.8 (d, 2H), 4.2 (m, 1H), 5.9 (br s, 1H), 6.6 (d, 1H), 6.7 (s, 1H), 7.0 (d, 1H), 7.15 (m, 2H), 7.4 (m, 2H), MS: 437.

### Example 11

### (R/S)-1-{[(4-{[(2,5-Dimethylbenzyl)oxy]methyl}piperidin-1-yl)sulphonyl]methyl}-4-pyrimidin-2-ylbutyl(hydroxy)formamide

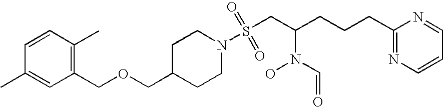

The procedure described in Example 1 was followed using 4-(2,5-dimethylbenzyloxymethyl)piperidin-1-ylsulphonylmethane (synthesis described below) (171 mg, 0.36 mmol) in place of 4-(2-methylquinolin-4-ylmethyloxy)piperidin-1-ylsulphonylmethane to give (R/S)-1-{[(4-{[(2,5-dimethylbenzyl)oxy]methyl}piperidin-1-yl)sulphonyl]methyl}-4-pyrimidin-2-ylbutyl(hydroxy)formamide (57 mg, 0.113 mmol). MS: 505.

The synthesis of 4-(2,5-dimethylbenzyloxymethyl)piperidin-1-ylsulphonylmethane was achieved as shown below:

i) To a stirred solution of 2,5-dimethylbenzyl alcohol (500 μl, 3.7 mmol) in DMF (15 ml) was added sodium hydride (180 mg, 4.9 mmol) at RT. After 90 minutes a solution of 4-(methanesulphonyloxymethyl)piperidin-1-ylsulphonylmethane (1.09 g, 4.0 mmol) in DMF (15 ml) was added and the reaction left to stir overnight at RT. Water (5 ml) was added and the solvent evaporated. The residue was then partitioned between EtOAc (40 ml) and brine (25 ml) and the aqueous layer re-extracted with EtOAc (25 ml). The combined organic layer was dried (Na₂SO₄), concentrated and purified by chromatography (Flashmaster II) to give 4-(2,5-dimethylbenzyloxymethyl)piperidin-1-ylsulphonylmethane as a white solid (400 mg, 1.3 mmol). NMR: 1.37 (m, 2H), 1.76 (m, 1H), 1.87 (m, 2H), 2.28 (s, 3H), 2.31 (s, 3H), 2.64 (m, 2H), 2.76 (s, 3H), 3.35 (d, 2H), 3.8 (m, 2H), 4.45 (s, 2H), 7.0-7.09 (m, 3H).

### Example 12

### (R/S)-2-Methyl-3-({4-[(2-methylquinolin-4-yl)methoxy]piperidin-1-yl}sulphonyl)propionic hydroxamic acid

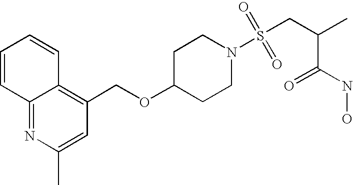

To a stirred solution of (R/S)-2-methyl-3-({4-[(2-methylquinolinyl)methoxy)piperidin-1-yl}sulphonyl)propionic acid (110 mg, 0.27 mmol) (described below) in DCM (4 ml) at 0° C. was added DMF (0.05 ml) and oxalyl chloride (0.03 ml, 0.32 mmol) dropwise. After 45 minutes at 0° C. the reaction mixture was concentrated in vacuo and dried under vacuum for 1 hour to give the acid chloride as a light brown foam. To a stirred solution of hydroxylamine (50% aqueous solution, 0.3 ml) in THF (5 ml) at RT was added a solution of the acid chloride in DCM (2 ml) dropwise over 5 minutes. After 1 hour the reaction mixture was diluted with EtOAc (20 ml) and treated with saturated ammonium chloride (10 ml). The organic layer was dried (Na₂SO₄), concentrated, triturated with ether, filtered and dried in vacuo to give (R/S)-2-methyl-3-({4-[(2-methylquinolin-4-yl)methoxy]piperidin-1-yl}sulphonyl)propionic hydroxamic acid as an off-white solid (77 mg, 0.18 mmol). Melting Point: 184.7° C.; NMR: 1.1 (d, 3H), 1.7 (m, 2H), 1.95 (m, 2H), 2.65 (s, 3H), 3.0 (m, 3H), 3.4 (m, 4H), 3.7 (m, 1H), 5.0 (s, 2H), 7.45 (s, 1H), 7.5 (t, 1H), 7.7 (t, 1H), 7.9 (d, 1H), 8.05 (d, 1H), 8.8 (s, 1H) and 10.55 (br s, 1H); MS: 422.5.

The (R/S)-2-methyl-3-({4-[(2-methylquinolin-4-yl)methoxy]piperidin-1-yl}sulphonyl)propionic acid described above was prepared as follows:

i) To a stirred suspension of 4-(2-methylquinolin-4-ylmethyloxy)-piperidin-1-ylsulphonylmethane (400 mg, 1.2 mmol) (synthesis described above) in THF (4 ml) at −10° C. under argon was added LHMDS (1.26 ml, 1M in THF, 1.26 mmol). Immediately after this to a separate stirred solution of 2-bromopropionic acid (0.12 ml, 1.26 mmol) in THF (4 ml) at −10° C. under argon was added LHMDS (1.32 ml, 1M in THF, 1.32 mmol). After 10 minutes this solution was added to the first solution over 5 minutes. After 1 hour saturated ammonium chloride (10 ml) was added, the reaction mixture acidified with glacial acetic acid and extracted with EtOAc (2×30 ml). The combined organic extracts were dried (Na₂SO₄), concentrated and azeotroped with toluene to give a brown gum. This was then purified by chromatography (10 g silica bond elute, eluent 0→10% EtOH in DCM) to give (R/S)-2-methyl-3-({4-[(2-methylquinolin-4-yl)methoxy]piperidin-1-yl}sulphonyl)propionic acid as a yellow foam (280 mg, 0.69 mmol). NMR: 1.25 (d, 3H), 1.7 (m, 2H), 2.0 (m, 2H), 2.65 (s, 3H), 2.8 (m, 1H), 3.05 (m, 3H), 3.4 (m, 3H), 3.7 (m, 1H), 5.0 (s, 2H), 7.4 (s, 1H), 7.5 (t, 1H), 7.7 (t, 1H), 7.9 (d, 1H), 8.05 (d, 1H); MS: 407.

### Example 13

### (R/S)-2-({4-[(2,5-difluorobenzyl)oxy]piperidin-1-yl}sulphonyl)-1-phenylethyl(hydroxy)formamide

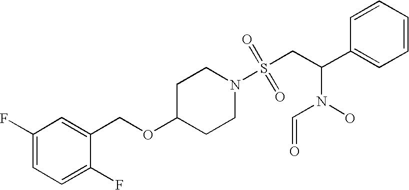

To a solution of (R/S)-2-{[4-[(2,5-difluorobenzyl)oxy]piperidin-1-yl]sulphonyl}-1-phenylethylhydroxylamine (described below) (0.75 mmol) in DCM (1 ml) was added a preformed-mixture of formic acid (2 ml) and acetic anhydride (1 ml) and stirred at RT overnight. MeOH (5 ml) was then added and, after stirring for 30 minutes, the mixture was evaporated. The residue was re-dissolved in MeOH (2 ml) and allowed to stand at RT overnight. After evaporation, the mixture was purified by chromatography (10 g silica bond elute, eluent 0→5% MeOH/DCM) to give (R/S)-2-({4-[(2,5-difluorobenzyl)oxy]piperidin-1-yl}sulphonyl)-1-phenylethyl(hydroxy)formamide (0.075 mmol, 34 mg) as a solid. MS: 455.

The starting (R/S)-2-{[4-[(2,5-difluorobenzyl)oxy]piperidin-1-yl]sulphonyl}-1-phenylethylhydroxylamine was prepared as follows:

i) Triethylamine (8.0 g, 0.079 mol) was added to a stirred solution of E-β-styrenesulphonyl chloride (12.0 g, 0.059 mol) and 4-hydroxypiperidine (8.0 g, 0.079 mol) in THF (100 ml) at RT. Stirring was continued overnight before the reaction mixture was concentrated to low volume and partitioned between EtOAc followed by aqueous 1M HCl, saturated sodium hydrogen carbonate and brine. The organic fraction was then dried (Na₂SO₄) and concentrated to give E-1-[4-hydroxypiperidin-1-ylsulphonyl]-2-phenylethene. (12.75 g; 0.046 mol); NMR (CDCl₃): 1.5-1.8 (m, 4H), 1.9-2.1 (m, 2H), 3.0-3.2 (m, 2H), 3.4-3.6 (m, 2H), 3.85 (s, 1H), 6.65 (s, 1H) and 7.3-7.6 (m, 6H); MS: 268.

ii) E-1-[4-hydroxypiperidin-1-ylsulphonyl]-2-phenylethene was dissolved in DMF (0.2 g; 0.75 mmol in 3 ml) and 2,5-difluorobenzyl bromide (1.5 mmol) was added. A covering of argon gas was introduced before solid sodium hydride (0.1 g; incl. oil) was carefully added, in three portions to the stirred reaction. Stirring was continued overnight. Water (5 ml) was added (dropwise initially) and the resultant mixture extracted with EtOAc (5 ml). The organic layer was separated and the aqueous layer washed again with EtOAc (3 ml). The combined organics were evaporated, re-dissolved in DCM (5 ml) and applied to a 10 g Silica BondElut column and eluted with a gradient from DCM to 2.5% MeOH in DCM to give E-1-[4-[(2,5-difluorobenzyl)oxy]piperidin-1-ylsulphonyl]-2-phenylethene which was carried through to the next step.

iii) E-1-[4-[(2,5-difluorobenzyl)oxy]piperidin-1-ylsulphonyl]-2-phenylethene, was dissolved in THF (1 ml) and the air in the tube excluded with argon before hydroxylamine in water (50% solution, 1 ml) was added and the mixture stirred vigorously overnight. EtOAc (1 ml) was added and the aqueous layer separated. The organic layers were washed with brine and dried (Na₂SO₄) and concentrated to give (R/S)-2-{[4-[(2,5-difluorobenzyl)oxy]piperidin-1-yl]sulphonyl}-1-phenylethylhydroxylamine which was carried through to the final step.

### Examples 14-31

The procedure described in Example 13 was repeated using the appropriate halide (bromo or chloro) in place of 2,5-difluorobenzyl bromide to give the products listed below.

| 1 | | Halide starting | |
|---|---|---|---|
| Example | Structure and Name | material | MH+ |
| 14 | 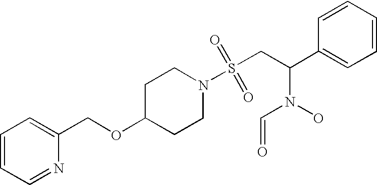 | 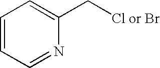 | 420 |
| | (R/S)-hydroxy(1-phenyl-2-{[4- | | |
| | (pyridin-2-ylmethoxy)piperidin-1- | | |
| | yl[sulphonyl}ethyl)formamide | | |
| 15 | 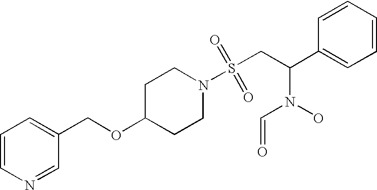 | 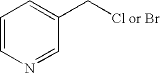 | 420 |
| | (R/S)-hydroxy(1-phenyl-2-{[4- | | |
| | (pyridin-3-ylmethoxy)piperidin-1- | | |
| | yl]sulphonyl}ethylformamide | | |
| 16 | 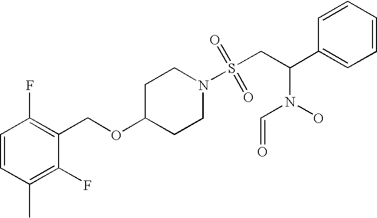 | 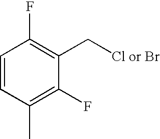 | 469 |
| | (R/S)-2-({4-[(2,6-difluoro-3- | | |
| | methylbenzyl)oxy]piperidin-1- | | |
| | yl}sulphonyl)-1- | | |
| | phenylethyl(hydroxy)formamide | | |
| 17 | 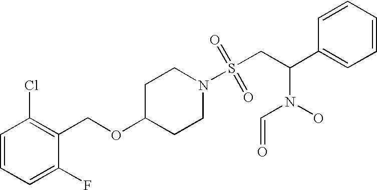 | 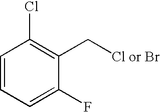 | 471 |
| | (R/S)-2-({4-[(2-chloro-6- | | |
| | fluorobenzyl)oxy]piperidin-1- | | |
| | yl}sulphonyl)-l- | | |
| | phenylethyl(hydroxy)formamide | | |
| 18 | 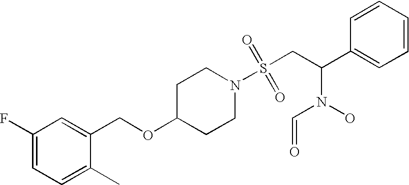 | 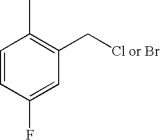 | 451 |
| | (R/S)-2-({4-[(5-fluoro-2- | | |
| | methylbenzyl)oxy]piperidin-1- | | |
| | yl}sulphonyl)-1- | | |
| | phenylethyl(hydroxy)formamide | | |
| 19 | 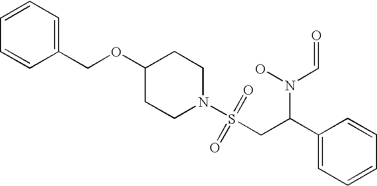 | 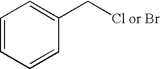 | 419 |
| | (R/S)-2-{[4-(benzyloxy)piperidifl-1- | | |
| | yl]sulphonyl}-1- | | |
| | phenylethyl(hydroxy)formamide | | |
| 20 | 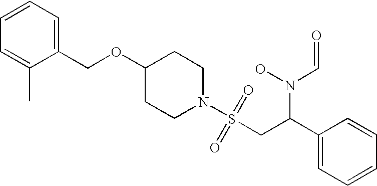 | 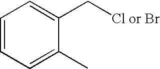 | 433 |
| | (R/S)-hydroxy[2-({4-[(2- | | |
| | methylbenzyl)oxy]pipendin-1- | | |
| | yl}sulphonyl)-1- | | |
| | phenylethyl)formamide | | |
| 21 | 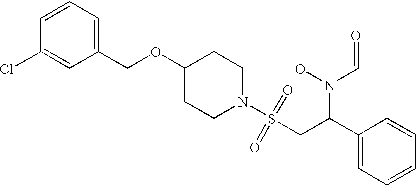 | 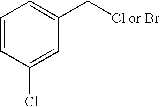 | 453 |
| | (R/S)-2-({4-[(3- | | |
| | chlorobenzyl)oxy]piperidin-1- | | |
| | yl}sulphonyl)-1- | | |
| | phenylethyl(hydroxy)formamide | | |
| 22 | 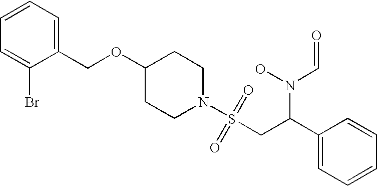 | 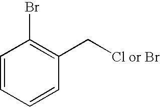 | 497 |
| | (R/S)-2-({4-[(2- | | |
| | bromobenzyl)oxy]piperidin-1- | | |
| | yl}sulphonyl)-1- | | |
| | phenylethyl(hydroxy)formamide | | |
| 23 | 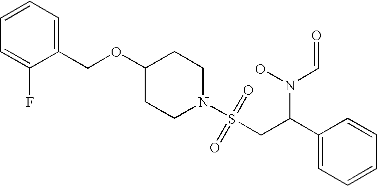 | 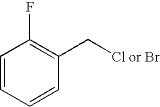 | 437 |
| | (R/S)-2-({4-[(2- | | |
| | fluorobenzyl)oxy]piperidin-1- | | |
| | yl}sulphonyl)-1- | | |
| | phenylethyl(hydroxy)formamide | | |
| 24 | 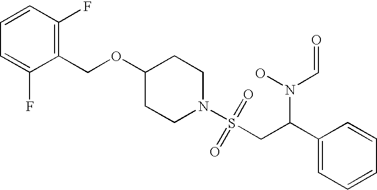 | 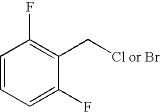 | 455 |
| | (R/S)-2-({4-[(2,6- | | |
| | difluorobenzyl)oxy]piperidin-1- | | |
| | yl}sulphonyl)-1- | | |
| | phenylethyl(hydroxy)formamide | | |
| 25 | 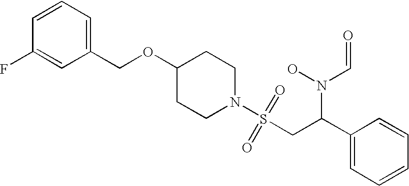 | 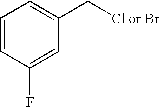 | 437 |
| | (R/S)-2-({4-[(3- | | |
| | fluorobenzyl)oxy]piperidin-1- | | |
| | yl}sulphonyl)-1- | | |
| | phenylethyl(hydroxy)formamide | | |
| 26 | 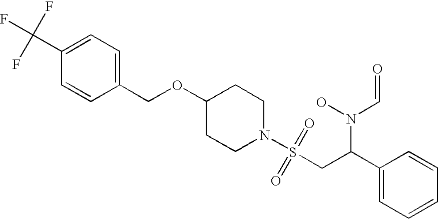 | 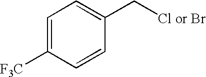 | 487 |
| | (R/S)-hydroxy{1-phenyl-2-[(4-{[4- | | |
| | (trifluoromethyl)benzyl]oxy}piperidin- | | |
| | 1-yl)sulphonyl]ethyl}formamide | | |
| 27 | 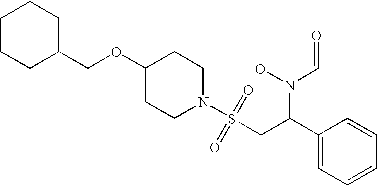 | 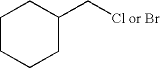 | 425 |
| | (R/S)-2-{[4- | | |
| | (cyclohexylmethoxy)piperidin-1- | | |
| | yl]sulphonyl}-1- | | |
| | phenylethyl(hydroxy)formamide | | |
| 28 | 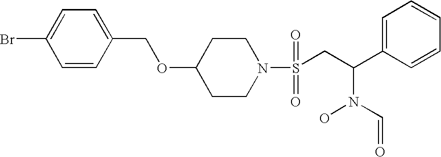 | 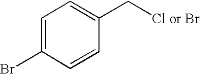 | 497 |
| | (R/S)-2-({4-[(4- | | |
| | bromobenzyl)oxy]piperidin-1- | | |
| | yl}sulphonyl)-1- | | |
| | phenylethyl(hydroxy)formamide | | |
| 29 | 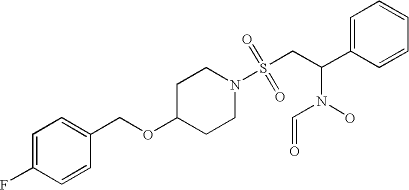 | 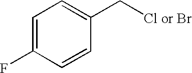 | 437 |
| | (R/S)-2-({4-[(4- | | |
| | fluorobenzyl)oxy]piperidin-1- | | |
| | yl}sulphonyl)-1- | | |
| | phenylethyl(hydroxy)formamide | | |
| 30 | 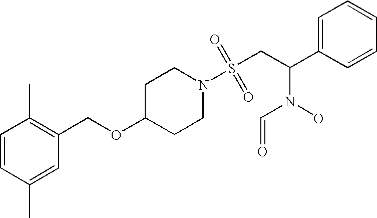 | 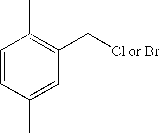 | 447 |
| | (R/S)-2-({4-[(2,5- | | |
| | dimethylbenzyl)oxy]piperidin-1- | | |
| | yl}sulphonyl)-1- | | |
| | phenylethyl(hydroxy)formamide | | |
| 31 | 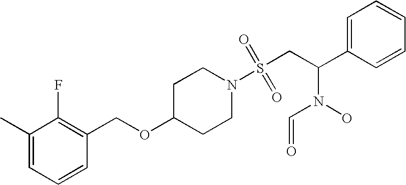 | 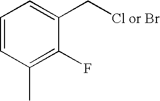 | 451 |
| | (R/S)-2-({4-[(2-fluoro-3- | | |
| | methylbenzyl)oxy]piperidin-1- | | |
| | yl}sulphonyl)-1- | | |
| | phenylethyl(hydroxy)formamide | | |

### Examples 32-45

The procedure described in Example 13 was repeated using the appropriate halide (bromo or chloro) in place of 2,5-difluorobenzyl bromide and using E-1-[4-hydroxypiperidin-1-ylsulphonyl]-2-(pyrid-3-yl)ethene (described below) instead of E-1-[4-hydroxypiperidin-1-ylsulphonyl]-2-phenylethene to give the products listed.

The synthesis of the starting E-1-[4-hydroxypiperidin-1-ylsulphonyl]-2-(pyrid-3-yl)ethene is shown below:

i) A solution of 4-hydroxypiperidine (8 g; 0.08 mol) in DCM (80 ml) was cooled in an ice bath before pyridine (7.4 ml; 0.09 mol) and TBDMS triflate (20 ml; 0.088 mol) were added. The resulting mixture was stirred for 2.5 hours. Iced water was added and the organic layer separated, washed with brine, dried and evaporated to give 4-(tert-butyldimethylsilyl)oxypiperidine as a pale yellow residue (24 g).

ii) Methanesulphonyl chloride (1.0 ml; 0.012 mol) was added to a solution of the 4-(tert-butyldimethylsilyl)oxypiperidine (2.7 g; 0.012 mol) and DIPEA (4.4 ml; 0.025 mol) in DCM (20 ml) and the whole stirred at RT overnight. Water (20 ml) was added and the organic layer separated and washed with 2M hydrochloric acid, saturated sodium bicarbonate and brine and evaporated to give 1-methanesulphonyl-4-(tert-butyldimethylsilyl)oxypiperidine as a black, oily residue.

iii) A solution of 1-methanesulphonyl-4-(tert-butyldimethylsilyl)oxypiperidine (2.0 g; 6.8 mmol) in THF (50 ml) was covered with argon and cooled in an ice/acetone bath before a solution of LHMDS in THF (15.0 ml; 1M; 15.0 mmol) was added dropwise. After stirring for 30 minutes, diethyl chlorophosphate (1.0 ml; 6.8 mmol) was added and stirring continued for a further 50 minutes. Nicotinaldehyde (0.64 ml; 6.8 mmol) was then added and the solution allowed to warn to RT and stirred overnight. A saturated solution of ammonium chloride was then added and the mixture extracted with EtOAc. The dried organic extracts were concentrated in vacuo. Purification was by chromatography on silica, eluting with a increasing gradient from hexane to 50% EtOAc in hexane to give E-1-[4(tert-butyldimethylsilyloxy)piperidin-1-ylsulphonyl]-2-(pyrid-3-yl)ethene (1.93 g, 5.05 mmol).

iv) E-1-[4-(tert-butyldimethylsilyloxy)piperidin-1-ylsulphonyl]-2-(pyrid-3-yl)ethene (1.93 g; 5.05 mmol) was added to a preformed-mixture of acetyl chloride (2 ml) in MeOH (20 ml) and stirred at RT for 2 hours. Concentration in vacuo gave a solid which was partitioned between saturated sodium bicarbonate and EtOAc. The organic extracts were dried and evaporated. Purification was by chromatography on silica (20 g) eluting with a gradient from DCM to 20% MeOH in DCM. Evaporation of fractions containing product gave E-1-[4-(hydroxy)piperidin-1-ylsulphonyl]-2-(pyrid-3-yl)ethene as a white solid (0.4 g; 30%). NMR (400 MHz): d, 1.4 (2H, m, CH2); 1.8 (2H, m, CH₂); 2.9 (2H, m, CH₂); 3.2 (2H, m, CH₂); 3.6 (1H, m, CH); 4.8 (1H, d, OH); 7.5 (3H, m, CH); 8.2 (1H, m, CH); 8.6 (1H, m, CH); 8.9 (1H, d, CH).

| | | Halide starting | |
|---|---|---|---|
| Example | Structure and Name | material | MH+ |
| 32 | 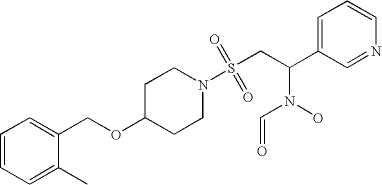 | 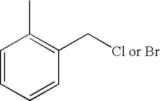 | 434 |
| | (R/S)-hydroxy[2-({4-[(2- | | |
| | methylbenzyl)oxy]piperidin-1- | | |
| | yl}sulphonyl)-1-pyridin-3- | | |
| | ylethyl]formamide | | |
| 33 | 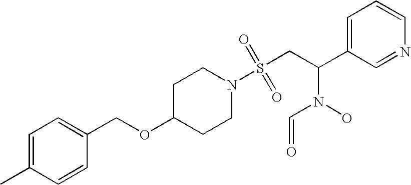 | 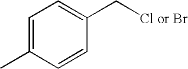 | 434 |
| | (R/S)-hydroxy[2-({4-[(4- | | |
| | methylbenzyl)oxy]piperidin-1- | | |
| | yl}sulphonyl)-1-pyridin-3- | | |
| | ylethyl]formamide | | |
| 34 | 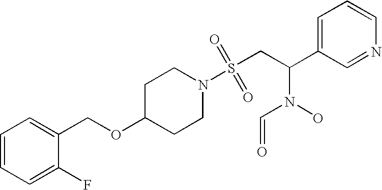 | 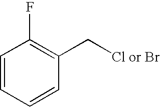 | 438 |
| | (R/S)-2-({4-[(2- | | |
| | fluorobenzyl)oxy]piperidin-1- | | |
| | yl}sulphonyl)-1-pyridin-3- | | |
| | ylethyl(hydroxy)formamide | | |
| 35 | 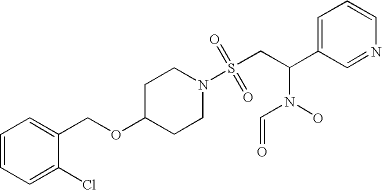 | 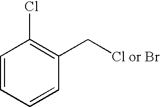 | 454 |
| | (R/S)-2-({4-[(2- | | |
| | chlorobenzyl)oxy]piperidin-1- | | |
| | yl}sulphonyl)-1-pyridin-3- | | |
| | ylethyl(hydroxy)formamide | | |
| 36 | 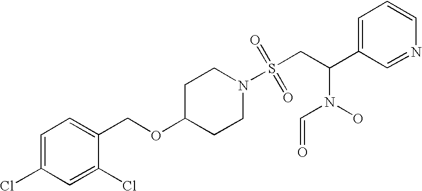 | 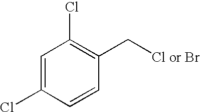 | 488 |
| | (R/S)-2-({4-[(2,4- | | |
| | dichlorobenzyl)oxy]piperidin-1- | | |
| | yl}sulphonyl)-1-pyridin-3- | | |
| | ylethyl(hydroxy)formamide | | |
| 37 | 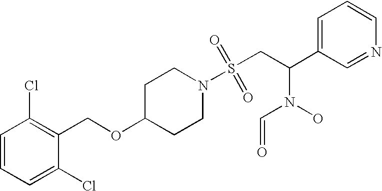 | 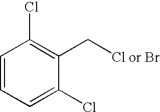 | 488 |
| | (R/S)-2-({4-[(2,6- | | |
| | dichlorobenzyl)oxy]piperidin-1- | | |
| | yl}sulphonyl)-1-pyridin-3- | | |
| | ylethyl(hydroxy)formamide | | |
| 38 | 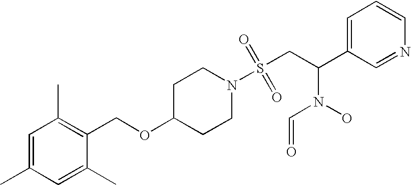 | 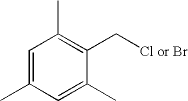 | 462 |
| | (R/S)-hydroxy(2-{[4- | | |
| | (mesitylmethoxy)piperidin-1- | | |
| | yl]sulphonyl}-1-pyridin-3- | | |
| | ylethyl)formamide | | |
| 39 | 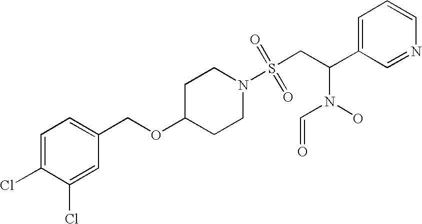 | 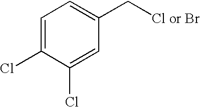 | 487 |
| | (R/S)-2-({4-[(3,4- | | |
| | dichlorobenzyl)oxy]piperidin-1- | | |
| | yl}sulphonyl)-1-pyridin-3- | | |
| | ylethyl(hydroxy)formamide | | |
| 40 | 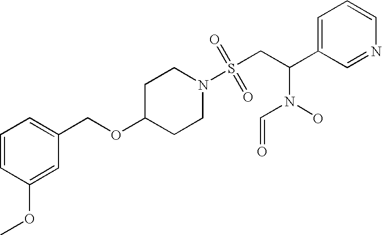 | 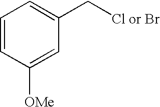 | 450 |
| | (R/S)-hydroxy[2-({4-[(3- | | |
| | methoxybenzyl)oxy]piperidin-1- | | |
| | yl}sulphonyl)-1-pyridin-3- | | |
| | ylethyl]formamide | | |
| 41 | 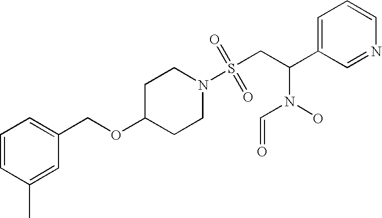 | 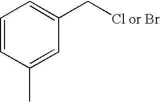 | 434 |
| | (R/S)-hydroxy[2-({4-{(3- | | |
| | methylbenzyl)oxy]piperidin-1- | | |
| | yl}sulphonyl)-1-pyridin-3- | | |
| | ylethyl]formamide | | |
| 42 | 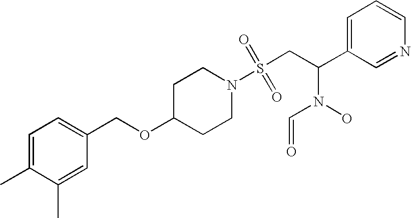 | 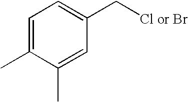 | 447 |
| | (R/S)-2-({4-[(3,4- | | |
| | dimethylbenzyl)oxy]piperidin-1- | | |
| | yl}sulphonyl)-1-pyridin-3- | | |
| | ylethyl(hydroxy)formamide | | |
| 43 | 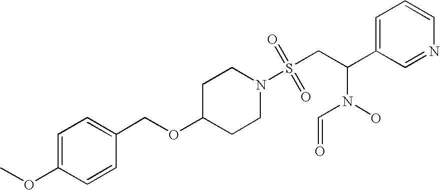 | 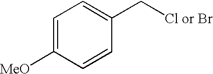 | 450 |
| | (R/S)-hydroxy[2-({4[(4- | | |
| | methoxybenzyl)oxy]piperidin-1- | | |
| | yl}sulphonyl)-1-pyridin-3- | | |
| | ylethyl]formamide | | |
| 44 | 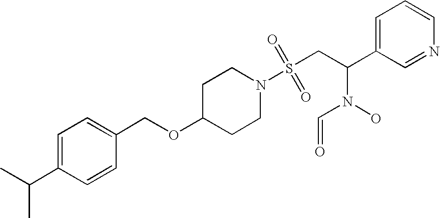 | 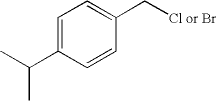 | 462 |
| | (R/S)-hydroxy[2-({4-[(4- | | |
| | isopropylbenzyl)oxy]piperidin-1- | | |
| | yl}sulphonyl)-1-pyridin-3- | | |
| | ylethyl]formamide | | |
| 45 | 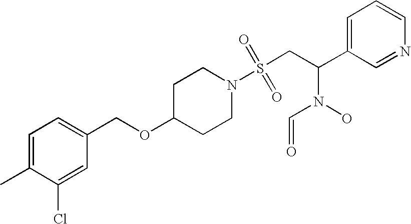 | 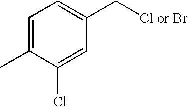 | 468 |
| | (R/S)-2-({4-[(3-chloro-4- | | |
| | methylbenzyl)oxy]piperidin-1- | | |
| | yl}sulphonyl)-1-pyridin-3- | | |
| | ylethyl(hydroxy)formamide | | |

### Example 46

### (R/S)—N-hydroxy-N-isopropyl-2-methyl-3-({4-[(2-methylquinolin-4-yl)methoxy]piperidin-1-yl}sulphonyl)propanamide

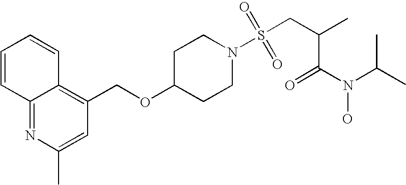

To a stirred solution of (R/S)-2-methyl-3-({4[(2-methylquinolin-4-yl)methoxy]piperidin-1-yl}sulphonyl)propionic acid (125 mg) (described within Example 12) in DCM (4 ml) under argon was added DMP (0.03 ml). The solution was cooled to 0° C. and oxalyl chloride (0.36 ml) was added in a dropwise manner. The temperature was maintained at 0° C. for 45 minutes and the solution then concentrated in vacuo and dried under vacuum for ten minutes. The resulting brown gum was dissolved in DCM (2 ml) and added to a vigorously stirred solution of N-isopropylhydroxylamine hydrochloride (173 mg) and triethylamine (0.22 ml) in THF (5 ml) dropwise over 2 minutes. Stirring was continued for 1 hour after which the reaction mixture was diluted with EtOAc (20 ml) and treated with saturated sodium chloride (10 ml), dried (sodium sulphate), concentrated in vacuo and purified on a 10 g silica bond elute using a 0-5% EtOH/DCM gradient over 40 minutes as eluent to give (R/S)—N-hydroxy-N-isopropyl-2-methyl-3-({4-[(2-methylquinolin-4-yl)methoxy]piperidin-1-yl}sulphonyl)propanamide as a pale yellow foam (43 mg). NMR: δ 1.05 (d, 6H), 1.15 (d, 3H), 1.7 (m, 2H), 1.95 (m, 2H), 2.65 (s, 3H), 2.9 (m, 1H), 3.0 (m, 2H), 3.4 (m, 4H+H₂O), 3.7 (m, 1H), 4.5 (m, 1H), 5.0 (s, 2H), 7.4 (s, 1H), 7.5 (t, 1H), 7.7 (t, 1H), 7.9 (d, 1H), 8.05 (d, 1H), 9.4 (s, 1H); MS: 464.28.

### Examples 47 and 48

Hydroxy{(1R)-1-[({4-[(2-methylquinolin-4-yl)methoxy]piperidin-1-yl}sulphonyl)methyl]-4-pyrimidin-2-ylbutyl}formamide and hydroxy{(1S)-1-[({4-[(2-methylquinolin-4-yl)methoxy]piperidin-1-yl}sulphonyl)methyl]-4-pyrimidin-2-ylbutyl}formamide

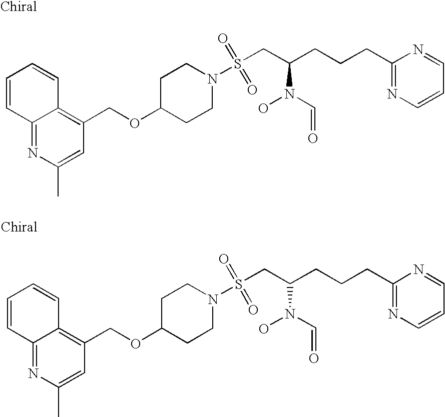

(R/S)-hydroxy{1-[((4-[(2-methylquinolin-4-yl)methoxy]piperidin-1-yl}sulphonyl)methyl]-4-pyrimidin-2-ylbutyl}formamide (472 mg) (described within Example 1) was enantiomerically separated by chiral chromatography (instrument: Rainin; column: Kromasil 100 10 μm CHI-TBB (250 mm×20 mm) ref E5009; eluent: MTBE/EtOAc/formic acid/triethylamine 1600/400/2/1) to give Isomer A, hydroxy{(1R)-1-[({4-[(2-methylquinolin-4-yl)methoxy]piperidin-1-yl}sulphonyl)methyl]-4-pyrimidin-2-ylbutyl}formamide, as a white foam (104 mg); NMR: δ 1.7(m, 6H), 2.0(m, 2H), 2.65(s, 3H), 2.85(m, 2H), 3.1(m, 3H), 3.4(m, 4H+H₂O), 3.7(m, 1H), 5.0(s, 2H), 7.3(m, 1H), 7.4(s, 1H), 7.5(t, 1H), 7.7(t, 1H), 8.1(m, 3H); 8.7(m, 2H); 9.7(m, 1H); MS: 527.98.

The same enantiomeric separation described in above also gave the other enantiomer, hydroxy{(1S)-1-[({4-[(2-methylquinolin-4-yl)methoxy]piperidin-1-yl}sulphonyl)methyl]-4-pyrimidin-2-ylbutyl}formamide, (isomer B) as a white foam (77 mg); NMR: δ 1.7(m, 6H), 2.0(m, 2H), 2.65(s, 3H), 2.85(m, 2H), 3.1(m, 3H), 3.4(m, 4H+H₂O), 3.7(m, 1H), 5.0(s, 2H), 7.3(m, 1H),7.4(s, 1H), 7.5(t, 1H), 7.7(t, 1H), 8.1(m, 3H); 8.7(m, 2H); 9.7(m, 1H), MS: 527.98.

### Example 49

### (2R)—N-hydroxy-2-methyl-3-({4-[(2-methylquinolin-4-yl)methoxy]piperidin-1-yl}sulphonyl)propanamide

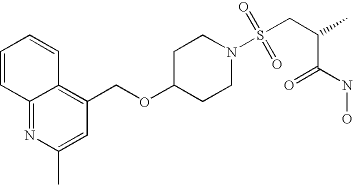

To a stirred solution of (R)-2-methyl-3-({4-[(2-methylquinolin-4-yl)methoxy]piperidin-1-yl}sulphonyl)propionic acid (120 mg) (described below) in DCM (5 ml) at RT was added DMF (0.05 ml). Oxalyl chloride (0.076 ml) was added. After 45 minutes the solution was concentrated in vacuo and dried under vacuum for 10 minutes. The residue was dissolved in DCM (3 ml) and added to a vigorously stirred solution of 50% aqueous hydroxylamine (0.5 ml) in THF (5 ml) dropwise over 2 minutes. Stirring was continued for 1 hour after which the reaction mixture was diluted with EtOAc (30 ml) and treated with saturated aqueous ammonium chloride (10 ml), dried (sodium sulphate) and concentrated in vacuo. The crude product was purified on a 10 g silica bond elute using a 0-30% ETOH/DCM gradient over 1 hour as eluent to give the product as a white foam (90 mg); MS: 422.24. Chiral purity determination (instrument Jasco, column 10 μm Kromasil 100 CHI-DMB(250×4.6), eluent TBME/acetic acid/triethylamine 100/0.1/0.05)≧99%.

The starting (R)-2-methyl-3-({4-[(2-methylquinolin-4-yl)methoxy]piperidin-1-yl}sulphonyl)propionic acid was prepared according to the same procedure in Example 12 for the synthesis of (R/S)-2-methyl-3-({4-[(2-methylquinolin-4-yl)methoxy]piperidin-1-yl}sulphonyl)propionic acid except that S-(−)-2-bromopropionic acid (0.085 ml) was used in place of 2-bromopropionic acid to give (R)-2-methyl-3-({4-[(2-methylquinolin-4-yl)methoxy]piperidin-1-yl}sulphonyl)propionic acid as a pale yellow foam (120 mg); MS 407.35.

### Examples 50, 51 and 52

(R/S)-2-Cyclopentyl-N-hydroxy-3-({4-[(2-methylquinolin-4-yl)methoxy1piperidin-1-yl}sulphonyl)propanamide, (2S)-2-cyclopentyl-N-hydroxy-3-({4-[(2-methylquinolin-4-yl)methoxy]piperidin-1-yl}sulphonyl)propanamide and (2R)-2-cyclopentyl-N-hydroxy-3-({4-[(2-methylquinolin-4-yl)methoxy]piperidin-1-yl}sulphonyl)propanamide

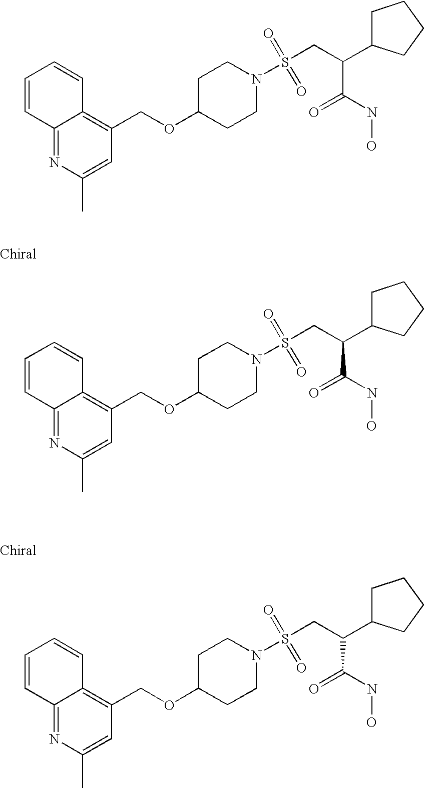

The method described in Example 49 was followed except that R)-2-methyl-3-({4-[(2-methylquinolin-4-yl)methoxy]piperidin-1-yl}sulphonyl)propionic acid was replaced with (R/S)-3-{[4-(2-methylquinolin-4-ylmethoxy)piperidin-1-yl]sulphonyl}-2-cyclopentylpropionic acid to give (R/S)-2-cyclopentyl-N-hydroxy-3-({4-[(2-methylquinolin-4-yl)methoxy]piperidin-1-yl}sulphonyl)propanamide as a white solid (210 mg). NMR: δ 1.2(m, 2H), 1.6(m, 9H), 2.0(m, 2H), 2.3(t, 1H), 2.65(s, 3H), 3.0(m, 3H), 3.4(m, 3H+H₂O), 3.7(m, 1H), 5.0(s, 2H), 7.4(s, 1H), 7.5(t, 1H), 7.7(t, 1H), 7.8(d, 1H), 8.05(d, 1H), 8.75(s, 1H), 10.5(s, 1H); MS: 476.11.

(R/S)-2-cyclopentyl-N-hydroxy-3-({4-[(2-methylquinolin-4-yl)methoxy]piperidin-1-yl}sulphonyl)propanamide (100 mg) was enantiomerically separated by chiral chromatography (instrument: Gilson, column: Merck 50 mm 20 μm Chiralpak AD No.ADOOSC-H001, eluent: acetonitrile/EtOH/acetic acid 95/5/0.2) to give isomer A, (2R)-2-cyclopentyl-N-hydroxy-3-({4-[(2-methylquinolin-4-yl)methoxy]piperidin-1-yl}sulphonyl)propanamide as a white solid (26 mg); MS 476.01. The method also yielded the other enantiomer (isomer B) (2S)-2-cyclopentyl-N-hydroxy-3-({4-[(2-methylquinolinyl)methoxy]piperidin-1-yl}sulphonyl)propanamide as a white solid (28mg); MS 476.03.

The starting (R/S)-3-{[4-(2-methylquinolin-4-ylmethoxy)piperidin-1-yl]sulphonyl}-2-cyclopentylpropionic acid was prepared as described below:

i) Sodium metal (2.88 g) was added in small portions to absolute ethanol (220 ml) under argon with stirring at RT. On achievement of complete solution, a mixture of diethyl malonate (20 g) and cyclopentyl bromide (18.64 g) was added and the mixture stirred under reflux for 2 hours, allowed to cool and excess solvent removed in vacuo. The residue was partitioned between water (150 ml) and diethyl ether (3×200 ml) and combined organics were dried (sodium sulphate), concentrated in vacuo and purified on a 10 g silica bond elute using a 5-35% EtOAc/isohexane gradient over 50 minutes as eluent to give diethyl cyclopentylmalonate as a colourless oil (18.34 g); NMR: δ 1.1(m, 9H), 1.5(m, 4H), 1.7(m, 2H), 2.3(m, 1H), 4.1 (q, 4H).

ii) 3M aqueous sodium hydroxide (200 ml) was added to a stirred solution of diethyl cyclopentylmalonate (18.33 g) in THF (300 ml) and MeOH (300 ml). Stirring was continued overnight and organic solvents removed in vacuo. The resulting aqueous solution was saturated with salt, acidified with concentrated hydrochloric acid and partitioned three times with EtOAc. The combined organic extracts were dried (magnesium sulphate), concentrated in vacuo and azeotroped once with toluene to give cyclopentylmalonic acid as an off-white solid (12.7 g); NMR: δ 1.2(m, 2H), 1.5(m, 4H), 1.7(m, 2H), 2.25(m, 1H), 3.0(d, 1H), 12.5(s, 2H); MS 171.18(ES−).

iii) Morpholine (7.08 ml) was added to a stirred solution of cyclopentylmalonic acid (12.69 g) in water (55 ml) and acetic acid (9 ml) at RT. After 20 minutes 37% aqueous formaldehyde (3.33 g) was added and stirring was continued overnight. The reaction was then heated to 80° C. and maintained for 2 hours, allowed to cool to RT and made basic with solid sodium hydrogen carbonate. This solution was washed with DCM (100 ml) and then acidified using 2M hydrochloric acid followed by concentrated hydrochloric acid and partitioned with DCM (3×150 ml). Combined organic extracts were washed with water (100 ml) and brine (100 ml), dried (magnesium sulphate) and concentrated in vacuo to give 2-cyclopentylpropenoic acid as a white solid (2.8 g); NMR: δ 1.3(m, 2H), 1.6(m, 4H), 1.8(m, 2H), 2.85(m,1H), 5.5(s, 1H), 6.0(s, 1H), 12.3(s, 1H); MS 139.11(ES−).

iv) Hydrogen bromide (30 wt. % solution in acetic acid, 22 ml) was added to 2-cyclopentylpropenoic acid (2.8 g). The mixture was stirred at RT overnight and then poured cautiously into water (130 ml) and partitioned with EtOAc (3×75 ml). The combined organic extracts were treated with water (50 ml) and brine (50 ml), dried (magnesium sulphate), concentrated in vacuo and azeotroped twice with toluene. The crude product was purified on a 50 g silica bond elute using a 25-50% EtOAc/isohexane gradient over 45 minutes as eluent to give (R/S)-3-bromo-2-cyclopentylpropionic acid as a pale yellow solid (2.51 g); NMR: δ 1.2(m, 2H), 1.6(m, 6H), 1.9(m, 1H), 2.5(m, 1H), 3.6(m, 2H); MS 223.23(ES+), 221.15(ES−).

v) (R/S)-3-Bromo-2-cyclopentylpropionic acid (2.5 g) was mixed with DCM (35 ml), isobutylene (18 ml) and concentrated sulphuric acid (2 drops) and the reaction was carried out at 25° C. for 48 hours at a pressure of 1 bar (high pressure facility). The solution was treated with saturated aqueous sodium hydrogen carbonate (50 ml), dried and concentrated in vacuo to give (R/S)-tert-butyl-3-bromo-2-cyclopentylpropionate as a light green oil (1.2 g); NMR: δ 1.3 (m, 3H), 1.4 (s, 9H), 1.6(m, 5H), 1.9 (m, 1H), 2.5(m, 1H), 3.6(m, 2H); MS: 278 (ES+).

vi) Potassium thioacetate (1.23 g) was added to a stirred solution of (R/S)-tert-butyl-3-bromo-2-cyclopentylpropionate (1.19 g) in DMP (25 ml) under argon at RT. The solution was heated to 100° C. and maintained for 3 hours, then allowed to cool, poured into water (100 ml) and partitioned with EtOAc (3×150 ml). Combined organic extracts were treated with saturated aqueous sodium hydrogen carbonate (50 ml), water (50 ml) and brine (50 ml), dried (sodium sulphate), concentrated in vacuo and purified on a 20 g silica bond elute using a 0-10% EtOAc/isohexane gradient over 45 minutes as eluent to give (R/S)-tert-butyl-3-acetylthio-2-cyclopentylpropionate as a light brown oil (870 mg); NMR: δ 1.2(m, 31), 1.4(s, 9H), 1.5(m, 4H), 1.8(m, 1H), 1.9(m, 1H), 2.2(m, 1H), 2.3(s, 3H), 3.0(m, 2H); MS 273(ES+), 271(ES−).

vii) (R/S)-Tert-butyl-3-acetylthio-2-cyclopentylpropionate (860 mg) was suspended in 5% acetic acid in water (50 ml) and stirred at RT. Chlorine gas was bubbled through the suspension for 30 minutes. The chlorine source was then removed and the reaction stirred for a further 30 minutes. The mixture was partitioned with DCM (3×100 ml) and combined organic extracts treated with water (50 ml) and brine (50 ml), dried (magnesium sulphate), concentrated in vacuo and azeotroped once with toluene to give (R/S)-tert-butyl-3-chlorosulphonyl-2-cyclopentylpropionate as a pale yellow oil (930 mg); NMR (CDCl₃) δ 1.4(m, 2H), 1.5(s, 9H), 1.7(m, 6H), 2.1(m, 1H), 2.9(m, 1H) and 3.9(m, 2H); MS: 296.61(ES+).

viii) (2-Methyl)quinolin-4-ylmethyloxypiperid-4-yl tetratrifluoroacetate (1.103 g) (described in example 1) in DCM (20 ml) was stirred under argon at 0° C. Triethylamine (1.52 ml) was added followed by a solution of tert-butyl-3-chlorosulphonyl-2-cyclopentylpropionate (460 mg) in DCM (2 ml). The reaction was maintained at 0° C. for 1 hour and then diluted with DCM (75 ml), washed with water (40 ml) and brine (40 ml), dried (sodium sulphate), concentrated in vacuo and purified on a 20 g silica bond elute using a 0-5% EtOH/DCM gradient as eluent to give (R/S)-tert-butyl-3-{(4-(2-methylquinolin-4-ylmethoxy)piperidin-1-yl]sulphonyl}-2-cyclopentylpropanoate as a pale yellow oil (470 mg), NMR: δ 1.2(m, 2H), 1.4(s, 9H), 1.6(m, 9H), 2.0(m, 3H), 2.65(s, 3H), 3.1(m, 3H), 3.4(m, 3H+H₂O), 3.7(m, 1H), 5.0(s, 2H), 7.4(s, 1H), 7.5(t, 1H), 7.7(t, 1H), 7.9(d, 1H), 8.0(d, 1H); MS 517.46(ES+).

ix) MeOH saturated with hydrogen chloride (10 ml) and concentrated hydrochloric acid (2 ml) were added to a solution of (R/S)-tert-butyl-3-{[4-(2-methylquinolin-4-ylmethoxy)piperidin-1-yl]sulphonyl}-2-cyclopentylpropanoate (460 mg) in MeOH (4 ml). The solution was stirred at RT for 2 hours, concentrated in vacuo and purified on a 20 g silica bond elute using a 0-10% EtOH/DCM gradient over 45 minutes as eluent to give (R/S)-3-{[4-(2-methylquinolin-4-ylmethoxy)piperidin-1-yl]sulphonyl}-2-cyclopentylpropionic acid as a white foam (360 mg); NMR: δ 1.2(m, 2H), 1.6(m, 8H), 2.0(m, 3H), 2.75(s, 3H), 3.1(m, 4H), 3.4(m, 3H+H₂O), 3.7(m, 1H), 5.1(s, 2H), 7.6(m, 2H), 7.8(t, 1H), 8.1(t, 2H); MS 461.2.

### Examples 53 and 54

(2S)—N-hydroxy-4-methyl-2-[({4-[(2-methylquinolin-4-yl)methoxy]piperidin-1-yl}sulphonyl)methyl]pentanamide and (2R)—N-hydroxy-4-methyl-2-[({4-[(2-methylquinolin-4-yl)methoxy]piperidin-1-yl}sulphonyl)methyl]pentanamide

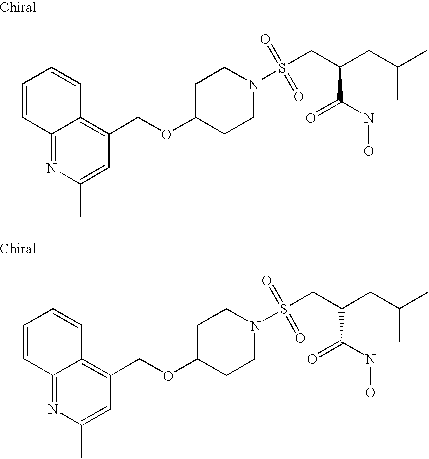

The method described in Example 49 was followed except that (R)-2-methyl-3-({4-[(2-methylquinolin-4-yl)methoxy]piperidin-1-yl}sulphonyl)propionic acid was replaced with (R/S)-4-methyl-2-[({4-[(2-methylquinolin-4-yl)methoxy]piperidin-1-yl}sulphonyl)methyl]pentanoic acid to give (R/S)—N-hydroxy-4-methyl-2-[({4-[(2-methylquinolin-4-yl)methoxy]piperidin-1-yl}sulphonyl)methyl]pentanamide as a white solid (69 mg); NMR: 0.8(m, 6H), 1.4(m, 3H), 1.65(m, 2H), 2.0(m, 2H), 2.7(s, 3H), 3.0(m, 3H), 3.5(m, 4H+H₂O); 3.7(m, 1H), 5.0(s, 2H), 7.4(s, 1H), 7.5(t, 1H), 7.7(t, 1H), 7.9(d, 1H), 8.05(d, 1H), 8.8(s,1H); MS: 461.84(ES−).

(R/S)—N-hydroxy-4-methyl-2-[({4-[(2-methylquinolin-4-yl)methoxy]piperidin-1-yl}sulphonyl)methyl]pentanamide (240 mg) was enantiomerically separated by chiral chromatography (instrument: Gilson, column: Merck 50 mm 20 μm Chiralpak AD No.ADOOSC-HBU01, eluent: acetonitrile/EtOH/acetic acid 95/5/0.2) to give isomer A, (R)—N-hydroxymethyl-2-[({4-[(2-methylquinolin-4-yl)methoxy]piperidin-1-yl}sulphonyl)methyl]pentanamide as a white solid (97 mg); MS: 464.2. The method also yielded the other enantiomer (isomer B) (R)—N-hydroxy-4-methyl-2-[({4-[(2-methylquinolin-4-yl)methoxy]piperidin-1-yl}sulphonyl)methyl]pentanamide as a white solid (89 mg); MS: 464.2.

The starting (R/S)-4-methyl-2-[({4-[(2-methylquinolin-4-yl)methoxy]piperidin-1-yl}sulphonyl)methyl]pentanoic acid was prepared as follows: i) The method described in Example 12 was followed except that 2-bromopropionic acid was replaced by dl-α-bromoisocaproic acid (185 mg) to give (R/S)-4-methyl-2-[({4-[(2-methylquinolin-4-yl)methoxy]piperidin-1-yl}sulphonyl)methyl]pentanoic acid as a pale yellow foam (320 mg); MS: 448.89.

### Example 55

### (R/S)—N-{1-[4-(2,6-Dimethyl-pyridin-4-ylmethoxy)-piperidine-1-sulphonylmethyl]-4-pyrimidin-2-yl-butyl}-N-hydroxy-formamide

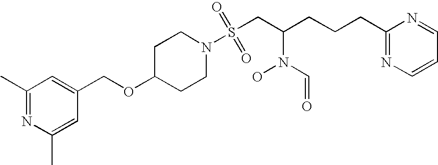

The procedure described in Example 1 was followed using 4-(1-methanesulphonylpiperidin-4-yloxymethyl)-2,6-dimethylpyridine (synthesis described below) (650 mg, 2.18 mmol) in place of (2-methylquinolin-4-ylmethyloxy)piperidin-4-ylsulphonylmethane to give ((R/S)—N-{1-[4-(2,6-Dimethylpyridin-4-ylmethoxy)piperidin-1-ylsulphonylmethyl}-4-pyrimidin-2-yl-butyl}-N-hydroxyformamide (120 mg, 0.24 mmol). NMR (CDCl₃): 1.67-1.97 (m, 8H), 2.52 (s, 6H), 2.88-3.55 (m, 8H), 3.60 (m, 1H), 4.21 (m, 0.5H), 4.46 (s, 1H), 4.47 (s 1H), 4.87 (m, 0.5H), 6.91 (s, 0.5H), 6.92 (s, 0.5H), 7.19 (t, 0.5H) 7.24 (t, 0.5H), 8.04 (s, 0.5H), 8.49 (s, 0.5H), 8.68 (d, 1H), 8.69 (d, 1H), 9.80 (s, 0.5H); MS: 492.

The synthesis of 4-(1-methanesulphonylpiperidin-4-yloxymethyl)-2,6-dimethylpyridine was achieved as shown below:

i) 4-Hydroxymethyl-2,6-lutidine^{¥} (800 mg, 5.83 mmol) was dissolved in chloroform. To this was added carbon tetrabromide (2.1 g, 6.4 mmol) and polystyrene supported triphenylphosphine (3 mmol/g, 4.3 g). The reaction mixture was stirred at RT for 20 minutesand then filtered. The solid was washed with DCM (5 oml) and the filtrate evaporated to a pale yellow oil. This was purified by chromatography (Flashmaster II, 50 g silica column, 0-100% EtOAcfisohexane)to give 4-bromomethyl-2,6-lutidine as a colourless oil (1.1 g, 5.5 mmol); NMR CDCl3) δ 2.52 (s, 6H), 4.32 (s, 2H), 6.97 (s, 2H).

ii) To a solution of 1-methanesulphonyl-piperidin-4-ol (700 mg, 3.9 mmol) in DMP (40 ml) was added sodium hydride (60% dispersion, 160 mg, 4.1 mmol). The suspension was stirred at RT for 20 minutes before adding a solution of 4-bromomethyl-2,6-lutidine (800mg, 4 mmol) in THF (7.3 ml). The reaction mixture was stirred at RT for 5 d. Water was added (2 ml) and the reaction mixture evaporated. The residue was partitioned between brine (40 ml) and EtOAc (40 ml). The aqueous phase was extracted with EtOAc (40 ml) and the combined organic phases dried (Na₂SO₄) and evaporated. Crude product was dissolved in EtOAc (20 ml) and extracted with 2M HCl (20 ml). The aqueous phase was basified with saturated aqueous and extracted with DCM (2×30 ml). The combined organic phases were dried and evaporated to give 4-(1-methanesulphonylpiperidin-4-yloxymethyl)-2,6-dimethylpyridine as a yellow oil which slowly solidified (650 mg, 2.18 mmol); NMR (CDCl3,) δ 1.87 (m, 2Et), 1.96 (m, 2H), 2.52 (s, 6H), 2.79 (s, 3H), 3.21 (m, 2H), 3.41 (m, 2H), 3.62 (1H, m), 4.48 (s, 2H), 6.92 (s,2H).

^{¥}This compound has been described in the literature: R. B. Katz, J. Mistry and M. B. Mitchell, Synth. Commun., 1989, 317-325.” on lines 25-26.

What we claim is:

1. A compound of formula (1): 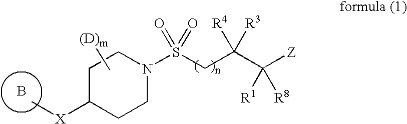
Z is selected from —CONR¹⁵OH and —N(OH)CHO;
R¹⁵ is hydrogen or C₁₋₃alkyl;
R¹ is hydrogen or a group selected from C₁₋₆alkyl, C₂₋₆alkenyl, C₂₋₆alkynyl, C₃₋₇cycloalkyl, C₅₋₇cycloalkenyl, aryl and heteroaryl where the group is optionally substituted by one or more substituents independently selected from halo, nitro, cyano, trifluoromethyl, trifluoromethyloxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, C₁₋₄alkoxycarbonyl, —OR⁵, —SR², —SOR², —SO₂R², —COR², —CO₂R⁵, —CONR⁵R, —NR¹⁶COR⁵, —SO₂NR⁵R⁶ and —NR¹⁶SO₂R²;
R¹⁶ is hydrogen or C₁₋₃alkyl;
R¹⁷ is selected from halo, nitro, cyano, trifluoromethyl, trifluoromethoxy, C₁₋₆alkyl, C₃₋₆cycloalkyl and C₁₋₆alkoxy;
R² is group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;
R⁵ is hydrogen or a group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;
R⁶ is hydrogen, C₁₋₆alkyl or C₃₋₆cycloalkyl;
or R⁵ and R⁶ together with the nitrogen to which they are attached form a heterocyclic 4- to 7-membered ring;
R⁵ is hydrogen or a group selected from C₁₋₆alkyl, C₃₋₇cycloalkyl and C₅₋₇cycloalkenyl where the group is optionally substituted by one or more substituents independently selected from halo, nitro, cyano, trifluoromethyl, trifluoromethyloxy and C₁₋₄alkyl;
R³ and R⁴ are both hydrogen;
n is 0 or 1;
m is 0 or 1;
D is hydrogen, C₁₋₄alkyl, C₃₋₆cycloalkyl or fluoro;
X is —(CR⁹R¹⁰)ₜ-Q-(CR¹¹R¹²)ᵤ— where t and u are independently 0 or 1 with the proviso that t and u cannot both be 0;
Q is O, S, SO or SO₂;
R⁹, R¹⁰, R¹¹ and R¹² are independently selected from hydrogen, C₁₋₄alkyl and C₃₋₆cycloalkyl;
B is a group selected from aryl, heteroaryl, heterocyclyl, C₃₋₁₀cycloalkyl and C₅₋₇cycloalkenyl where each group is optionally substituted by one or more groups independently selected from nitro, trifluoromethyl, trifluoromethyloxy, halo, C₁₋₄alkyl (optionally substituted by one or more R¹³), C₂₋₄alkenyl, C₂₋₄alkynyl, C₃₋₆cycloalkyl (optionally substituted by one or more R¹³), heterocycloalkyl, heteroaryl, aryl, —OR¹³, cyano, —NR¹³R¹⁴, —CONR¹³R¹⁴, —NR¹⁶COR¹³, —SO₂NR¹³R¹⁴, —NR¹⁶SO₂R¹³, —SR¹³, —SOR⁷ and —SO₂R⁷;
R⁷ is C₁₋₆alkyl or C₃₋₆cycloalkyl
R¹³ and R¹⁴ are independently hydrogen, C₁₋₆alkyl or C₃₋₆cycloalkyl;
or R¹³ and R¹⁴ together with the nitrogen to which they are attached form a heterocyclic 4 to 7-membered ring.
or a pharmaceutically acceptable salt or an in vivo hydrolysable ester thereof.

2. A compound according to claim 1 wherein X is —(CH₂)—O—, —O—(CH₂)—, —(CH₂)—O—(CH₂)— or —(CHMe)—O—.

3. A compound according to claim 1 wherein R¹ is C₁₋₄alkyl, C₂₋₄alkynyl, C₃₋₆cycloalkyl, aryl, heteroaryl and C₁₋₄alkyl substituted by aryl or heteroaryl wherein any R¹ group is optionally substituted by one or more substitutents independently selected from halo, cyano, nitro, C₁₋₄alkoxy, C₁₋₄alkyl, trifluoromethyl and trifluoromethoxy.

4. A compound according to claim 1 wherein B is a group selected from aryl, heteroaryl, heterocyclyl, C₃₋₁₀cycloalkyl and C₅₋₇cycloalkenyl where each group is optionally substituted by one or more groups independently selected from nitro, trifluoromethyl, halo, C₁₋₄alkyl, heteroaryl, —OR¹³, cyano, —NR¹³R¹⁴, —CONR¹³R¹⁴ and —NR¹⁶COR¹³.

5. A compound according to claim 4 wherein B is aryl, heteroaryl or C₃₋₆cycloalkyl optionally substituted by 1, 2 or 3 groups independently selected from C₁₋₄alkyl, halo, cyano, nitro, C₁₋₄alkoxy and trifluoromethyl

6. A compound according claim 5 wherein B is 2,5-dimethylphenyl or 2-methylquinolin-4-yl.

7. A compound according to claim 1, selected from:
(R/S)-1-[({4-[(2-methylquinolin-4-yl)methyloxy]piperidin-1-yl}sulphonyl)methyl]-4-pyrimidin-2-ylbutyl(hydroxy)formamide;
(R/S)-1-methyl-2-({4-[(2-methylquinolin-4-yl)methoxy]piperidin-1-yl}sulphonyl)ethyl(hydroxy)formamide;
(R/S)-1-pyrid-3-yl-2-({4-[(2-methylquinolin-4-yl)methoxy]piperidin-1-yl}sulphonyl)ethyl(hydroxy)formamide;
(R/S)-1-(1H-imidazol-4-yl)-2-({4-[(2-methylquinolin-4-yl)methoxy]piperidin-1-yl}sulphonyl)ethyl(hydroxy)formamide;
(R/S)-2-({4-[(2,5-dimethylbenzyl)oxy]piperidin-1-yl}sulphonyl)-1-pyrid-3-ylethyl(hydroxy)formamide;
(R/S)-[1-({[4-(2,5-dimethylbenzyloxy)piperidin-1-yl]sulphonyl}methyl)-3-phenylpropyl]hydroxyformamide;
(R/S)-2-({4-[(2,5-dimethylbenzyl)oxy]piperidin-1-yl}sulphonyl)-1-[4-fluoro-2-(trifluoromethyl)phenyl]ethyl(hydroxy)formamide;
(R/S)-2-({4-[(2,5-dimethylbenzyl)oxy]piperidin-1-yl}sulphonyl)-1-[2-(trifluoromethyl)phenyl]ethyl(hydroxy)formamide;
(R/S)-2-({4-[(2,5-dimethylbenzyl)oxy]piperidin-1-yl}sulphonyl)-1-[3-(trifluoromethyl)phenyl]ethyl(hydroxy)formamide;
(R/S)-2-({4-[(2,5-dimethylphenoxy)methyl]piperidin-1-yl}sulphonyl)-1-(4-fluorophenyl)ethyl(hydroxy)formamide;
(R/S)-1-{[(4-{[(2,5-dimethylbenzyl)oxy]methyl}piperidin-1-yl)sulphonyl]methyl}-4-pyrimidin-2-ylbutyl(hydroxy)formamide
(R/S)-2-methyl-3-({4-[(2-methylquinolin-4-yl)methoxy]piperidin-1-yl}sulphonyl)propionic hydroxamic acid
(R/S)-2-({4-[(2,5-difluorobenzyl)oxy]piperidin-1-yl}sulphonyl)-1-phenylethyl(hydroxy)formamide
(R/S)-hydroxy(1-phenyl-2-{[4-(pyridin-2-ylmethoxy)piperidin-1-yl]sulphonyl}ethyl)formamide;
(R/S)-hydroxy(1-phenyl-2-{[4-(pyridin-3-ylmethoxy)piperidin-1-yl]sulphonyl}ethyl)formamide;
(R/S)-2-({4-[(2,6-difluoro-3-methylbenzyl)oxy]piperidin-1-yl}sulphonyl)-1-phenylethyl(hydroxy)formamide;
(R/S)-2-({4-[(2-chloro-6-fluorobenzyl)oxy]piperidin-1-yl}sulphonyl)-1-phenylethyl(hydroxy)formamide;
(R/S)-2-({4-[(5-fluoro-2-methylbenzyl)oxy]piperidin-1-yl}sulphonyl)-1-phenylethyl(hydroxy)formamide;
(R/S)-2-{[4-(benzyloxy)piperidin-1-yl]sulphonyl}-1-phenylethyl(hydroxy)formamide;
(R/S)-hydroxy[2-({4-[(2-methylbenzyl)oxy]piperidin-1-yl}sulphonyl)-1-phenylethyl]formamide;
(R/S)-2-({4-[(3-chlorobenzyl)oxy]piperidin-1-yl}sulphonyl)-1-phenylethyl(hydroxy)formamide;
(R/S)-2-({4-[(2-bromobenzyl)oxy]piperidin-1-yl}sulphonyl)-1-phenylethyl(hydroxy)formamide;
(R/S)-2-({4-[(2-fluorobenzyl)oxy]piperidin-1-yl}sulphonyl)-1-phenylethyl(hydroxy)formamide;
(R/S)-2-({4-[(2,6-difluorobenzyl)oxy]piperidin-1-yl}sulphonyl)-1-phenylethyl(hydroxy)formamide;
(R/S)-2-({4-[(3-fluorobenzyl)oxy]piperidin-1-yl}sulphonyl)-1-phenylethyl(hydroxy)formamide;
(R/S)-hydroxy{1-phenyl-2-[(4-{[4-(trifluoromethyl)benzyl]oxy}piperidin-1-yl)sulphonyl]ethyl}formamide;
(R/S)-2-{[4-(cyclohexylmethoxy)piperidin-1-yl]sulphonyl}-1-phenylethyl(hydroxy)formamide;
(R/S)-2-({4-[(4-bromobenzyl)oxy]piperidin-1-yl}sulphonyl)-1-phenylethyl(hydroxy)formamide;
(R/S)-2-({4-[(4-fluorobenzyl)oxy]piperidin-1-yl}sulphonyl)-1-phenylethyl(hydroxy)formamide;
(R/S)-2-({4-[(2,5-dimethylbenzyl)oxy]piperidin-1-yl}sulphonyl)-1-phenylethyl(hydroxy)formamide;
(R/S)-2-({4-[(2-fluoro-3-methylbenzyl)oxy]piperidin-1-yl}sulphonyl)-1-phenylethyl(hydroxy)formamide;
(R/S)-hydroxy[2-({4-[(2-methylbenzyl)oxy]piperidin-1-yl}sulphonyl)-1-pyridin-3-ylethyl]formamide;
(R/S)-hydroxy[2-({4-[(4-methylbenzyl)oxy]piperidin-1-yl}sulphonyl)-1-pyridin-3-ylethyl]formamide;
(R/S)-2-({4-[(2-fluorobenzyl)oxy]piperidin-1-yl}sulphonyl)-1-pyridin-3-ylethyl(hydroxy)formamide;
(R/S)-2-({4-[(2-chlorobenzyl)oxy]piperidin-1-yl}sulphonyl)-1-pyridin-3-ylethyl(hydroxy)formamide;
(R/S)-2-({4-[(2,4-dichlorobenzyl)oxy]piperidin-1-yl}sulphonyl)-1-pyridin-3-ylethyl(hydroxy)formamide;
(R/S)-2-({4-[(2,6-dichlorobenzyl)oxy]piperidin-1-yl}sulphonyl)-1-pyridin-3-ylethyl(hydroxy)formamide;
(R/S)-hydroxy(2-{[4-(mesitylmethoxy)piperidin-1-yl]sulphonyl}-1-pyridin-3-ylethyl)formamide;
(R/S)-2-({4-[(3,4-dichlorobenzyl)oxy]piperidin-1-yl}sulphonyl)-1-pyridin-3-ylethyl(hydroxy)formamide;
(R/S)-hydroxy[2-({4-[(3-methoxybenzyl)oxy]piperidin-1-yl}sulphonyl)-1-pyridin-3-ylethyl]formamide;
(R/S)-hydroxy[2-({4-[(3-methylbenzyl)oxy]piperidin-1-yl}sulphonyl)-1-pyridin-3-ylethyl]formamide;
(R/S)-2-({4-[(3,4-dimethylbenzyl)oxy]piperidin-1-yl}sulphonyl)-1-pyridin-3-ylethyl(hydroxy)formamide;
(R/S)-hydroxy[2-({4-[(4-methoxybenzyl)oxy]piperidin-1-yl}sulphonyl)-1-pyridin-3-ylethyl]formamide;
(R/S)-hydroxy[2-({4-[(4-isopropylbenzyl)oxy]piperidin-1-yl}sulphonyl)-1-pyridin-3-ylethyl]formamide;
(R/S)-2-({4-[(3-chloro-4-methylbenzyl)oxy]piperidin-1-yl}sulphonyl)-1-pyridin-3-ylethyl(hydroxy)formamide;
(R/S)—N-hydroxy-N-isopropyl-2-methyl-3-({4-[(2-methylquinolin-4-yl)methoxy]piperidin-1-yl}sulphonyl)propanamide;
hydroxy{(1R)-1-[({4-[(2-methylquinolin-4-yl)methoxy]piperidin-1-yl}sulphonyl)methyl]-4-pyrimidin-2-ylbutyl}formamide;
hydroxy{(1S)-1-[({4-[(2-methylquinolin-4-yl)methoxy]piperidin-1-yl}sulphonyl)methyl]-4-pyrimidin-2-ylbutyl}formamide;
(2R)—N-hydroxy-2-methyl-3-({4-[(2-methylquinolin-4-yl)methoxy]piperidin-1-yl}sulphonyl)propanamide
(R/S)-2-cyclopentyl-N-hydroxy-3-({4-[(2-methylquinolin-4-yl)methoxy]piperidin-1-yl}sulphonyl)propanamide;
(2S)-2-cyclopentyl-N-hydroxy-3-({4-[(2-methylquinolin-4-yl)methoxy]piperidin-1-yl}sulphonyl)propanamide;
(2R)-2-cyclopentyl-N-hydroxy-3-({4-[(2-methylquinolin-4-yl)methoxy]piperidin-1-yl}sulphonyl)propanamide;
(2S)—N-hydroxy-4-methyl-2-[({4-[(2-methylquinolin-4-yl)methoxy]piperidin-1-yl}sulphonyl)methyl]pentanamide;
(2R)—N-hydroxy-4-methyl-2-[({4-[(2-methylquinolin-4-yl)methoxy]piperidin-1-yl}sulphonyl)methyl]pentanamide; and
(R/S)—N-{1-[4-(2,6-dimethyl-pyridin-4-ylmethoxy)-piperidine-1-sulphonylmethyl]-4-pyrimidin-2-yl-butyl}-N-(hydroxy)formamide.

8. (canceled)

9. A method, the method comprising treating a disease condition mediated by one or more metalloproteinase enzymes by administering to a warm-blooded animal in need of such treatment an effective amount of a compound according to claim 1.

10. A method, the method comprising treating a disease condition mediated by TNFα by administering to a warm-blooded animal in need of such treatment an effective amount of a compound according to claim 1.

11. A method of treating autoimmune disease, allergic/atopic diseases, transplant rejection, graft versus host disease, cardiovascular disease, reperfusion injury and malignancy in a warm-blooded animal, in need of such treatment which comprises administering to said animal an effective amount of a compound according to claim 1.

12. A pharmaceutical composition comprising a compound according to claim 1; and a pharmaceutically-acceptable diluent or carrier.

13. A process for preparing a compound according to claim 1 which comprises; when Z is —N(OH)CHO, the step of:
a) converting a hydroxylamine of formula (2) into a compound of formula (1); 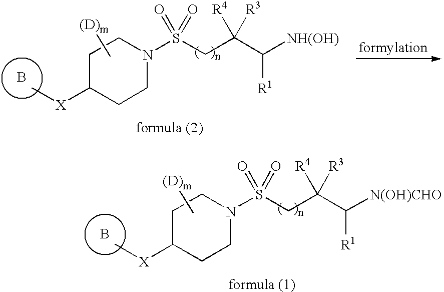
or where Z is —CONR¹⁵OH the step of;
b) converting an acid of formula (14) into a compound of formula (1); 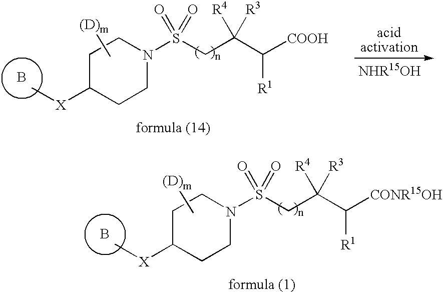
and thereafter if necessary:
i) converting a compound of formula (1) into another compound of formula (1);
ii) removing any protecting groups;
iii) forming a pharmaceutically acceptable salt or in vivo hydrolysable ester.
